(19) United States
(12) Patent Application Publication
(10) Pub. No.: US 2026/0371829 A1
(43) Pub. Date: Nov. 19, 2026

(54) PHOTOELECTRIC CONVERSION ELEMENT MATERIAL, AND PHOTOELECTRIC CONVERSION ELEMENT USING SAME

(71) Applicant: NIPPON STEEL Chemical & Material Co., Ltd., Tokyo (JP)

(72) Inventors: Kentaro HAYASHI, Tokyo (JP); Munetomo INOUE, Tokyo (JP)

(73) Assignee: NIPPON STEEL Chemical & Material Co., Ltd., Tokyo (JP)

(21) Appl. No.: 19/109,121

(22) PCT Filed: Aug. 31, 2023
(86) PCT No.: PCT/JP2023/031849

§ 371 Date: Mar. 5, 2025

(30) Foreign Application Priority Data
Sep. 14, 2022 (JP) 2022-145901

(51) Int. Cl. H10K 39/32; C07D 265/38; H10K 30/60; H10K 30/86; H10K 85/20; H10K 85/60

(52) U.S. Cl. CPC H10K 39/32; C07D 265/38; H10K 30/60; H10K 30/86; H10K 85/211; H10K 85/654; H10K 85/656; H10K 85/6572; H10K 85/6576; H10K 85/658

(57) ABSTRACT

Provided are a material that achieves higher sensitivity and higher resolution of a photoelectric conversion device for imaging, and a photoelectric conversion device for imaging using the above material. Used is a material for a photoelectric conversion device including a compound represented by the general formula (1). Ar¹ to Ar³ represent deuterium, an alkyl group having 1 to 20 carbon atoms, an arylheteroarylamino group having 12 to 36 carbon atoms, an aromatic hydrocarbon group having 6 to 18 carbon atoms, and the like, at least one of Ar¹ to Ar³ is represented by any of the general formulae (2) to (5) and the like.

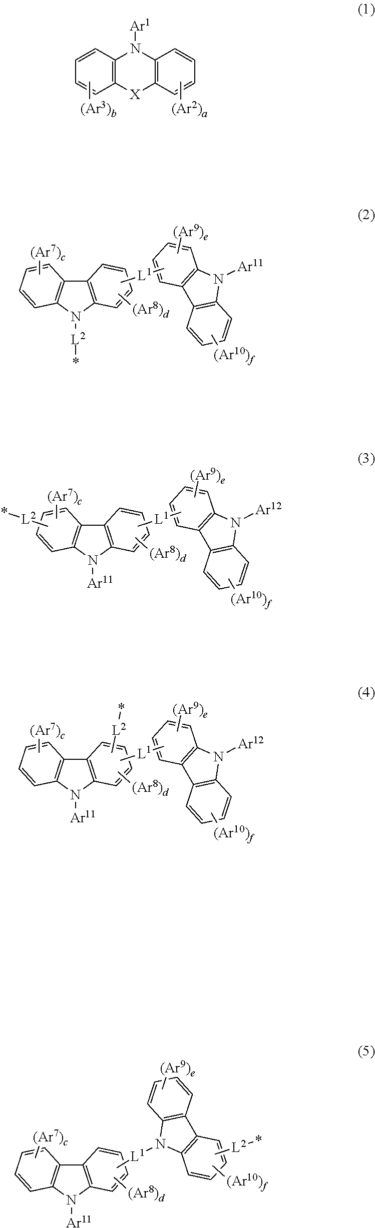

### TECHNICAL FIELD

The present invention relates to a material for a photoelectric conversion device and a photoelectric conversion device using the same, and particularly to a material for a photoelectric conversion device useful for an imaging device.

In recent years, development of an organic electronic device using a thin film formed with an organic semiconductor is in progress. Examples thereof include an electroluminescent device, a solar cell, a transistor device, and a photoelectric conversion device. In particular, development of an organic EL device, which is an electroluminescent device with an organic substance, is most advanced among them. The applications for smartphones, TV and the like are in progress, and development for a purpose of further higher functionality is continuously conducted.

Meanwhile, on the photoelectric conversion device for imaging, a device using a P-N junction of an inorganic semiconductor, such as silicon, has been conventionally developed and practically used, and made are investigations for high functionalization of a digital camera and a camera for a smartphone and investigation for application for a monitoring camera, a sensor for an automobile, and the like. However, problems for these various uses include improving sensitivity and micronizing a pixel (improving resolution). For the conventional photoelectric conversion device using an inorganic semiconductor, a mainly adopted method for obtaining a color image is disposing color filters corresponding to RGB, which are the three primary colors of light, on a light receiving part of the photoelectric conversion device. This method has problems in terms of utilization efficiency of an incident light and resolution, because the method disposes the RGB color filters on a plane (Non Patent Literature 1 and 2).

As a solution for such problems of the photoelectric conversion device, a photoelectric conversion device using an organic semiconductor instead of the inorganic semiconductor is developed (Non Patent Literature 1 and 2). This utilizes an ability to selectively absorb only light having a specific wavelength region with high sensitivity that the organic semiconductor has, and proposed is stacking photoelectric conversion devices composed of organic semiconductors corresponding to the three primary colors of light to solve the problem of improving the sensitivity and improving the resolution. A device in which a photoelectric conversion device composed of the organic semiconductor and a photoelectric conversion device composed of the inorganic semiconductor are stacked is also proposed (Non Patent Literature 3).

Here, the photoelectric conversion device composed of the organic semiconductor is a device having a photoelectric conversion layer composed of a thin film of the organic semiconductor between two electrodes, wherein a hole blocking layer and/or an electron blocking layer is disposed between the photoelectric conversion layer and the two electrodes, as necessary. In the photoelectric conversion device, light having a desired wavelength is absorbed in the photoelectric conversion layer to generate an exciton, and then charge separation of the exciton generates a hole and an electron. Thereafter, the hole and the electron move toward each electrode to convert the light into an electric signal. For a purpose of accelerating this process, a method of applying a bias voltage between both the electrodes is commonly used, but one of objects is reducing a leakage current from both the electrodes generated by applying the bias voltage. Accordingly, it can be mentioned that controlling the move of the hole and the electron in the photoelectric conversion device is a key to exhibit characteristic of the photoelectric conversion device.

The organic semiconductor used for each layer of the photoelectric conversion device can be classified into a P-type organic semiconductor and an N-type organic semiconductor. The P-type organic semiconductor is used as a hole transport material, and the N-type organic semiconductor is used as an electron transport material. To control the move of the hole and the electron in the photoelectric conversion device, made are various developments of an organic semiconductor having appropriate physical properties such as hole mobility, electron mobility, an energy value of a highest occupied molecular orbital (HOMO), and an energy value of a lowest unoccupied molecular orbital (LUMO). However, the organic semiconductor still has insufficient characteristics.

Various compounds that are excellent as an organic electroluminescent device or a hole transport material or an electron blocking material for an organic solar cell have been disclosed, but these compounds are not always excellent as a hole transport material or an electron blocking material for imaging. Specifically, as one important characteristic required for the hole transport material of the photoelectric conversion device for imaging, for example, it is important to increase a difference between a current value in a state of a closed shutter and a current value in a state of an opened shutter (a contrast ratio) in order to perform clear imaging with a camera in a dark place. Therefore, a lower current value in a dark place is more preferable, and a higher current value in a bright place is more preferable. That is, “high contrast ratio” is important.

Meanwhile, characteristics to be improvement targets in the organic electroluminescent device are a voltage required for flowing a constant current, luminescent efficiency, device lifetime, and the like, which are significantly different from the characteristics required in the photoelectric conversion device for imaging. In addition, it is an important for the organic solar cell to have a large quantity of current generation in both of the dark place and the bright place, which is different from the characteristics required in the photoelectric conversion device for imaging, and a lower current value in the dark place is more preferable.

As above, the required characteristics are different between: the hole transport material or the electron blocking material for the organic electroluminescent device or the organic solar cell; and the hole transport material or the electron blocking material for the photoelectric conversion device for imaging. Thus, it has not been clear until confirmation by experiment whether a material excellent as the hole transport material or the electron blocking material for the organic electroluminescent device or the organic solar cell is also excellent as the hole transport material or the electron blocking material for the photoelectric conversion device for imaging.

Here, Patent literature 1 proposes a device using quinacridone as the P-type organic semiconductor and subphthalocyanine chloride as the N-type organic semiconductor for the photoelectric conversion layer, and an indolocarbazole derivative for a first buffer layer disposed between the photoelectric conversion layer and the electrode.

In addition, Patent literature 2 proposes a device using, for the photoelectric conversion layer, a chrysenodithiophene derivative as the P-type organic semiconductor and fullerenes or a subphthalocyanine derivative as the N-type organic semiconductor.

Furthermore, Patent Literatures 3 and 4 propose a device using a carbazole derivative for an electron blocking layer disposed between the photoelectric conversion layer and the electrode.

Moreover, Patent Literature 5 proposes a device using a pyrene derivative or a triphenylene derivative for an electron blocking layer disposed between the photoelectric conversion layer and the electrode.

### CITATION LIST

### Patent Literature

### Patent Literature 1

JP 2018-85427 (A)

### Patent Literature 2

JP 2019-54228 (A)

### Patent Literature 3

JP 2011-228614 (A)

### Patent Literature 4

JP 2021-77888 (A)

### Patent Literature 5

JP 2015-153910 (A)

### Non Patent Literature

### Non Patent Literature 1

NHK Science & Technology Research Laboratories R&D No. 132, pp. 4-11 (2012.3)

### Non Patent Literature 2

NHK Science & Technology Research Laboratories R&D No. 174, pp. 4-17 (2019.3)

### Non Patent Literature 3

2019 IEEE International Electron Devices Meeting (IEDM), pp. 16.6.1-16.6.4 (2019)

### SUMMARY OF INVENTION

### Technical Problem

In the use of the photoelectric conversion device for imaging for highly functionalizing a digital camera and a camera for a smartphone and for application for a monitoring camera, a sensor for an automobile, and the like, challenges are further higher sensitivity and higher resolution. In view of such a circumstance, an object of the present invention is to provide a material that achieves higher sensitivity and higher resolution of the photoelectric conversion device for imaging, and a photoelectric conversion device for imaging using the same.

### Solution to Problem

The present inventors have made intensive investigation, and consequently found that using a specific compound efficiently advances a process of generating a hole and an electron by charge separation of an exciton in a photoelectric conversion layer, and a process of moving of the hole and the electron in the photoelectric conversion device. This finding has led to the completion of the present invention.

The present invention is a material for a photoelectric conversion device, the material comprising a compound represented by the following general formula (1).

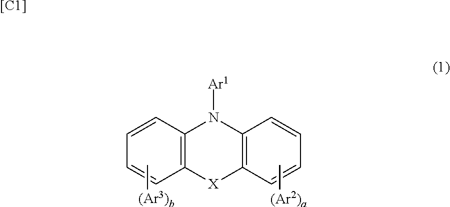

In the general formula (1), Ar¹ to Ar³ each independently represent deuterium, a cyano group, a halogen, a nitro group, an alkyl group having 1 to 20 carbon atoms, an aralkyl group having 7 to 38 carbon atoms, an alkenyl group having 2 to 20 carbon atoms, an alkynyl group having 2 to 20 carbon atoms, an acyl group having 2 to 20 carbon atoms, an acyloxy group having 2 to 20 carbon atoms, an alkoxy group having 1 to 20 carbon atoms, an alkoxycarbonyl group having 2 to 20 carbon atoms, an alkoxycarbonyloxy group having 2 to 20 carbon atoms, an alkylsulfonyl group having 1 to 20 carbon atoms, a dialkylamino group having 2 to 20 carbon atoms, a substituted or unsubstituted alkylarylamino group having 7 to 28 carbon atoms, a substituted or unsubstituted diarylamino group having 12 to 36 carbon atoms, a substituted or unsubstituted arylheteroarylamino group having 12 to 36 carbon atoms, a substituted or unsubstituted diheteroarylamino group having 12 to 36 carbon atoms, a substituted or unsubstituted aromatic hydrocarbon group having 6 to 18 carbon atoms, a substituted or unsubstituted aromatic heterocyclic group having 3 to 18 carbon atoms, or a substituted or unsubstituted linked aromatic group in which 2 to 10 aromatic rings of the aromatic hydrocarbon group or the aromatic heterocyclic group are linked, and at least one of Ar¹ to Ar³ is represented by any of the following general formulae (2) to (8). When Ar¹ to Ar³ represent a group having a hydrogen atom, the hydrogen atom is optionally replaced with deuterium or a halogen.

X represents C(Ar⁴Ar⁵), NAr⁶, O, or S.

“a” and “b” represent the number of substitutions, and each independently represent an integer of 0 to 4.

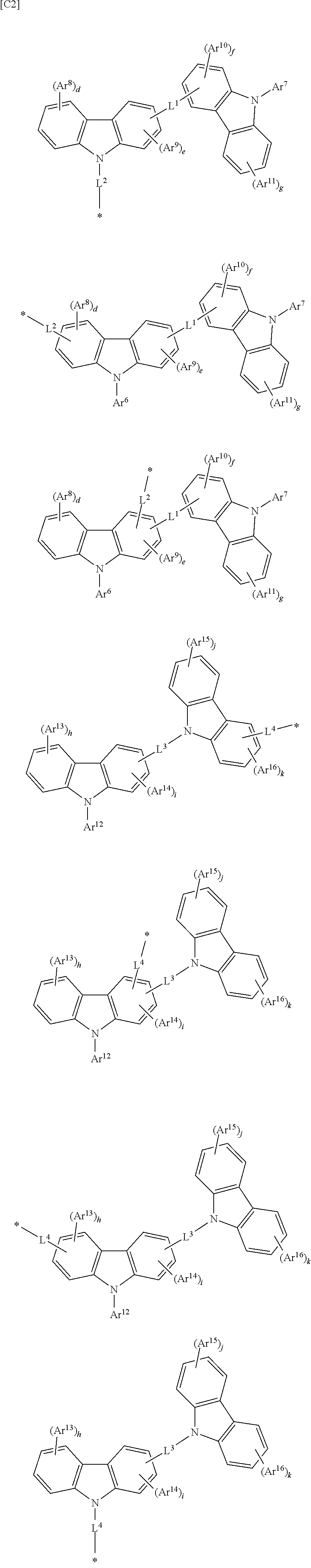

In the general formulae (2) to (8), “*” represents a bonding point to the general formula (1).

L¹ and L² each independently represent a direct bond, a substituted or unsubstituted aromatic hydrocarbon group having 6 to 18 carbon atoms, a substituted or unsubstituted aromatic heterocyclic group having 3 to 18 carbon atoms, or a substituted or unsubstituted linked aromatic group in which two to three aromatic rings of these groups are linked.

Ar⁴ to Ar¹² are each independently the same as described for Ar¹ to Ar³, provided that Ar⁴ to Ar¹², when representing the linked aromatic group, represent a substituted or unsubstituted linked aromatic group in which two to three aromatic rings are linked. Ar⁴ and Ar⁵ are optionally bonded to each other to form a cyclic structure.

“c” to “f” represent the number of substitutions, “c”, “e”, and “f” each independently represent an integer of 0 to 3, and “d” represents an integer of 0 to 2.

The general formula (1) is preferably represented by any of the following general formulae (9) to (12). The general formula (1) is more preferably represented by any of the following general formulae (9), (11), and (12), and further preferably represented by any of the following general formulae (11) and (12).

### [C3]

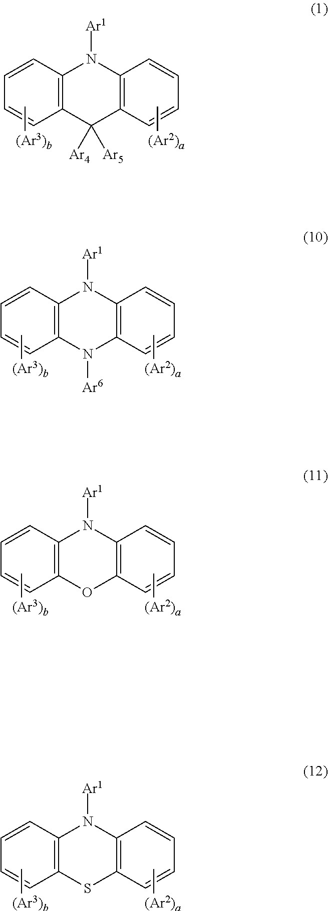

In the general formulae (9) to (12), Ar¹ to Ar⁶, “a”, and “b” are the same as described for the general formula (1).

At least one of Ar¹ to Ar³ in the general formula (1) is preferably represented by any of the general formulae (2) to (8), and more preferably represented by any of the general formulae (2), (3), (5), (7), and (8).

In the general formulae (2) to (8), L¹ preferably represents a single bond.

In the material for a photoelectric conversion device, an energy level of highest occupied molecular orbital (HOMO) obtained by structural optimization calculation with a density functional calculation B3LYP/6-31G(d) is preferably −4.0 eV or lower. An energy level of lowest unoccupied molecular orbital (LUMO) is preferably −2.5 eV or higher.

The material for a photoelectric conversion device preferably has a hole mobility of 1×10⁻⁶ cm²/Vs or more. The material for a photoelectric conversion device is preferably amorphous.

The material for a photoelectric conversion device may be used as a hole transport material.

The present invention is a photoelectric conversion device for imaging, comprising a photoelectric conversion layer and an electron blocking layer between two electrodes, wherein at least one layer of the photoelectric conversion layer or the electron blocking layer contains the above material for a photoelectric conversion device.

In the photoelectric conversion device of the present invention, the electron blocking layer can contain the material for a photoelectric conversion device, and the photoelectric conversion layer can contain an electron transport material such as a fullerene derivative.

### Advantageous Effect of Invention

Although the material for a photoelectric conversion device of the present invention can be applied for the photoelectric conversion device for imaging, the material for a photoelectric conversion device of the present invention can also be applied for a sensor converting other optical information into a signal, a solar cell, and the like. The material for a photoelectric conversion device of the present invention applied for the photoelectric conversion device for imaging can achieve appropriate move of the hole and the electron, and consequently enables to reduce a leakage current generated by applying a bias voltage during the conversion of light into electric energy. As a result, it is considered that a photoelectric conversion device that achieves a low dark current value and a high contrast ratio has been obtained. Therefore, the material of the present invention is useful as a material for a photoelectric conversion device for a photoelectric-converting film-stacked imaging device.

### BRIEF DESCRIPTION OF DRAWING

FIG. 1 is a sectional schematic view illustrating a structure example of a photoelectric conversion device used in the present invention.

### DESCRIPTION OF EMBODIMENTS

The photoelectric conversion device in the present invention is a photoelectric conversion device having at least one organic layer between two electrodes and converting light into electric energy. This organic layer contains the material for a photoelectric conversion device comprising the compound represented by the general formula (1). Specifically, in the photoelectric conversion device for imaging having the photoelectric conversion layer and the electron blocking layer between two electrodes, at least one layer of the photoelectric conversion layer and the electron blocking layer contains the material for a photoelectric conversion device represented by the general formula (1). Hereinafter, the material for a photoelectric conversion device composed of the compound represented by the general formula (1) is simply referred to as “material for a photoelectric conversion device”, or may be referred to as “material of the present invention” or “compound represented by the general formula (1)”.

The compound represented by the general formula (1) will be described below.

The general formula (1) is represented by the general formulae (9) to (12) as preferable embodiments, preferably represented by the general formula (9), (11), or (12), and specifically, more preferably represented by the general formula (11) or (12).

In these general formulae (9) to (12), Ar¹ to Ar⁶ and “a” and “b” are the same as described for the general formula (1).

In the general formula (1), at least one of Ar¹ to Ar³ is represented by any of the general formulae (2) to (8). Ar¹ is preferably represented by any of the general formulae (2) to (8).

In the general formula (1), at least one of Ar¹ to Ar³ is represented by any of the general formulae (2) to (8), preferably represented by any of the general formulae (2), (3), (5), (7), or (8), and more preferably represented by any of the general formulae (2), (5), or (8).

In the general formulae (2) to (8), “*” represents a bonding point to the general formula (1), and L¹, L², Ar⁴ to Ar¹², and “c” to “f” are the same as described for the general formulae (2) to (8).

Ar¹ to Ar³ each independently represent deuterium, a cyano group, a halogen, a nitro group, an alkyl group having 1 to 20 carbon atoms, an aralkyl group having 7 to 38 carbon atoms, an alkenyl group having 2 to 20 carbon atoms, an alkynyl group having 2 to 20 carbon atoms, an acyl group having 2 to 20 carbon atoms, an acyloxy group having 2 to 20 carbon atoms, an alkoxy group having 1 to 20 carbon atoms, an alkoxycarbonyl group having 2 to 20 carbon atoms, an alkoxycarbonyloxy group having 2 to 20 carbon atoms, an alkylsulfonyl group having 1 to 20 carbon atoms, a dialkylamino group having 2 to 20 carbon atoms, a substituted or unsubstituted alkylarylamino group having 7 to 28 carbon atoms, a substituted or unsubstituted diarylamino group having 12 to 36 carbon atoms, a substituted or unsubstituted arylheteroarylamino group having 12 to 36 carbon atoms, a substituted or unsubstituted diheteroarylamino group having 12 to 36 carbon atoms, a substituted or unsubstituted aromatic hydrocarbon group having 6 to 18 carbon atoms, a substituted or unsubstituted aromatic heterocyclic group having 3 to 18 carbon atoms, or a substituted or unsubstituted linked aromatic group in which 2 to 10 aromatic rings of the aromatic hydrocarbon group or the aromatic heterocyclic group are linked. Among these, Ar¹ to Ar³ preferably represent a substituted or unsubstituted aromatic hydrocarbon group having 6 to 18 carbon atoms, a substituted or unsubstituted aromatic heterocyclic group having 6 to 18 carbon atoms, or a substituted or unsubstituted linked aromatic group in which 2 to 10 aromatic rings of the aromatic hydrocarbon group or the aromatic heterocyclic group are linked. When these groups have a hydrogen atom in Ar¹ to Ar³, the hydrogen atom is optionally replaced with deuterium or a halogen. As noted above, at least one of Ar¹ to Ar³ is represented by any of the general formulae (2) to (8).

For Ar¹ to Ar³, the alkyl group having 1 to 20 carbon atoms may be any of linear, branched, and cyclic alkyl groups, and is preferably a linear, branched, or cyclic alkyl group having 1 to 10 carbon atoms. Specific examples thereof include: linear saturated hydrocarbon groups, such as a methyl group, an ethyl group, a n-propyl group, a n-butyl group, a n-pentyl group, a n-hexyl group, a n-octyl group, a n-dodecyl group, a n-tetradecyl group, and a n-octadecyl group; branched saturated hydrocarbon groups, such as an isopropyl group, an isobutyl group, a neopentyl group, a 2-ethylhexyl group, and a 2-hexyloctyl group; and saturated alicyclic hydrocarbon groups, such as a cyclopentyl group, a cyclohexyl group, a cyclooctyl group, and a 4-butylcyclohexyl group.

Specific examples of the aralkyl group having 7 to 38 carbon atoms include a benzyl group, a phenethyl group, a phenylpropyl group, a phenylbutyl group, a naphthylmethyl group, and a triphenylenylmethyl group. Specific examples of the alkenyl group having 2 to 20 carbon atoms include an ethylene group, a propylene group, a butylene group, a pentene group, a cyclopentene group, a hexene group, a cyclohexene group, and an octene group. Specific examples of the alkynyl group having 2 to 20 carbon atoms include an acetylene group, a propyne group, a butyne group, and a pentyne group. Specific examples of the acyl group having 2 to 20 carbon atoms include a formyl group, an acetyl group, a propionyl group, and a benzoyl group. Specific examples of the acyloxy group having 2 to 20 carbon atoms include a methanoyl group, an ethanoyl group, a propanoyl group, a butanoyl group, a benzoyl group, a propenoyl group, and a butenoyl group. Specific examples of the alkoxy group having 1 to 20 carbon atoms include a methoxy group, an ethoxy group, a propoxy group, and a butoxy group. Specific examples of the alkoxycarbonyl group having 2 to 20 carbon atoms include a methoxycarbonyl group, an ethoxycarbonyl group, a propoxycarbonyl group, a butoxycarbonyl group, and a pentoxycarbonyl group. Specific examples of the alkoxycarbonyloxy group having 2 to 20 carbon atoms include a methoxycarbonyloxy group, an ethoxycarbonyloxy group, a propoxycarbonyloxy group, a butoxycarbonyloxy group, and a pentoxycarbonyloxy group. Specific examples of the alkylsulfonyl group having 1 to 20 carbon atoms include methylsulfonyl, ethylsulfonyl, propylsulfonyl, butylsulfonyl, and pentylsulfonyl.

When Ar¹ to Ar³ represent the amino group, Ar¹ to Ar³ represent a dialkylamino group having 2 to 20 carbon atoms, a substituted or unsubstituted alkylarylamino group having 7 to 28 carbon atoms, a substituted or unsubstituted diarylamino group having 12 to 36 carbon atoms, a substituted or unsubstituted arylheteroarylamino group having 12 to 36 carbon atoms, or a substituted or unsubstituted diheteroarylamino group having 12 to 36 carbon atoms. Specific examples of the alkyl group include: linear saturated hydrocarbon groups, such as a methyl group, an ethyl group, a n-propyl group, a n-butyl group, a n-pentyl group, a n-hexyl group, and a n-octyl group; branched saturated hydrocarbon groups, such as an isopropyl group, an isobutyl group, a neopentyl group, and a 2-ethylhexyl group; and saturated alicyclic hydrocarbon groups, such as a cyclopentyl group, a cyclohexyl group, a cyclooctyl group, and a 4-butylcyclohexyl group. Specific examples of the aryl group include a phenyl group, a biphenyl group, a terphenyl group, a quaterphenyl group, a naphthyl group, a phenanthrenyl group, an anthracenyl group, a triphenylenyl group, a pyrenyl group, a fluorenyl group, and a spiro-bifluorenyl group. Specific examples of the heteroaryl group include groups generated from pyrrole, pyrrolopyrrole, indole, pyrroloindole, benzoindole, naphthopyrrole, isoindole, pyrroloisoindole, benzoisoindole, naphthoisopyrrole, carbazole, phenoxazine, phenothiazine, acridine, phenazine, benzocarbazole, indoloindole, indolocarbazole, carbazolocarbazole, benzofurocarbazole, benzothienocarbazole, carboline, thiophene, benzothiophene, naphthothiophene, dibenzothiophene, benzothienonaphthalene, benzothienobenzothiophene, benzothienodibenzothiophene, dinaphthothiophene, dinaphthothienothiophene, naphthobenzothiophene, furan, benzofuran, naphthofuran, dibenzofuran, benzofuronaphthalene, benzofurobenzofuran, benzofurodibenzofuran, dinaphthofuran, dinaphthofuranofuran, naphthobenzofuran, pyridine, pyrimidine, triazine, quinoline, isoquinoline, quinazoline, quinoxaline, and the like. Specific examples of the dialkylamino group include groups generated from a dimethylamino group, a methylethylamino group, a diethylamino group, a dipropylamino group, a di-n-butylamino group, a di-t-butylamino group, a dipentylamino group, a dihexylamino group, a dicyclohexylamino group, a diheptylamino group, a dioctylamino group, a dinonylamino group, a didecylamino group, and the like. Specific examples of the alkylarylamino group include groups generated from a methylphenylamino group, an ethylphenylamino group, a propylphenylamino group, a butylphenylamino group, a cyclohexylphenylamino group, a cyclohexylnaphthylamino group, a cyclohexylfluorenylamino group, a cyclohexylphenanthrenyl amino group, a cyclohexylfluoranthenylamino group, a cyclohexyltriphenylenylamino group, and the like. Specific examples of the diarylamino group include groups generated from a diphenylamino group, a phenylnaphthylamino group, a dinaphthylamino group, a phenylanthracenylamino group, a phenylfluorenylamino group, a phenylphenanthrenylamino group, a phenylfluoranthenylamino group, a phenyltriphenylenylamino group, a difluorenylamino group, a naphthyltriphenylenylamino group, a ditriphenylenylamino group, and the like. Specific examples of the arylheteroarylamino group include groups generated from a phenylpyridylamino group, a phenylindolyl group, a phenyldibenzofuranylamino group, a phenyldibenzothiophenylamino group, and the like. Specific examples of the diheteroarylamino group include groups generated from a bis-dibenzofuranylamino group, a bis-dibenzofuranylphenylamino group, a bis-carbazolylamino group, a dibenzofuranylcarbazolylamino group, a dibenzofuranyldibenzothiophenylamino group, a dibenzothiophenylcarbazolylamino group, and the like.

Examples of the unsaturated aromatic hydrocarbon group having 6 to 18 carbon atoms in Ar¹ to Ar³ include groups obtained by removing one hydrogen from a known aromatic hydrocarbon. Examples of such an aromatic hydrocarbon group include groups generated from: monocyclic aromatic hydrocarbons, such as benzene; bicyclic aromatic hydrocarbons, such as naphthalene; tricyclic aromatic hydrocarbons, such as indacene, biphenylene, phenalene, anthracene, phenanthrene, and fluorene; and tetracyclic aromatic hydrocarbons, such as fluoranthene, acephenanthrylene, aceanthrylene, triphenylene, pyrene, chrysene, tetraphene, tetracene, and pleiadene. The aromatic hydrocarbon group is preferably a group generated from benzene, naphthalene, anthracene, phenanthrene, triphenylene, or pyrene.

Examples of the unsubstituted aromatic heterocyclic group having 3 to 18 carbon atoms in Ar¹ to Ar³ include groups obtained by removing one hydrogen from an aromatic heterocyclic group. Examples of such an aromatic heterocyclic group include: a group generated from nitrogen-containing aromatic compounds having a pyrrole ring, such as pyrrole, pyrrolopyrrole, indole, pyrroloindole, benzoindole, naphthopyrrole, isoindole, pyrroloisoindole, benzoisoindole, naphthoisopyrrole, carbazole, benzocarbazole, indoloindole, carbazolocarbazole, indolocarbazole, and carboline; sulfur-containing aromatic compounds having a thiophene ring, such as thiophene, benzothiophene, naphthothiophene, phenothiazine, dibenzothiophene, benzothienonaphthalene, benzothienobenzothiophene, benzothienodibenzothiophene, dinaphthothiophene, dinaphthothienothiophene, naphthobenzothiophene, and benzothienocarbazole; oxygen-containing aromatic compounds having a furan ring, such as furan, benzofuran, naphthofuran, phenoxazine, dibenzofuran, benzofuronaphthalene, benzofurobenzofuran, benzofurodibenzofuran, dinaphthofuran, dinaphthofuranofuran, naphthobenzofuran, and benzofurocarbazole; pyridine, pyrimidine, triazine, quinoline, isoquinoline, quinazoline, and quinoxaline. The aromatic heterocyclic group is preferably a group generated from dibenzofuran, dibenzothiophene, carbazole, phenoxazine, phenothiazine, benzothienodibenzothiophene, benzofurodibenzofuran, benzofurocarbazole, or benzothienocarbazole.

Further, the unsaturated linked aromatic group in which aromatic groups of the aromatic hydrocarbon group and the aromatic heterocyclic group are linked herein refers to an aromatic group in which two or more aromatic groups are bonded and linked with a single bond. These linked aromatic groups may be linear or branched. A linking position in linking the benzene rings each other may be any of ortho, meta, and para, but para-liking or meta-linking is preferable. The aromatic group may be an aromatic hydrocarbon group or an aromatic heterocyclic group. The plurality of the aromatic groups may be same as or different from each other.

When Ar¹ to Ar³ represent the linked aromatic group, the number of linking is 2 to 10, preferably 2 to 8, and more preferably 2 to 6. The number of linking when Ar¹ to Ar³ represent the linked aromatic group is the total number of linking of two carbazole (CBZ) groups and the aromatic hydrocarbon group and the aromatic heterocyclic group included in L¹, L², and Ar⁶ to Ar¹² described in the formulae (2) to (8).

In the general formula (1) and the general formulae (9) to (12), “a” and “b” represent the number of substitutions, and each independently represent an integer of 0 to 4, preferably 0 to 2, and more preferably 0.

In the general formulae (2) to (8), L¹ and L² each independently represent a direct bond, a substituted or unsubstituted aromatic hydrocarbon group having 6 to 18 carbon atoms, a substituted or unsubstituted aromatic heterocyclic group having 3 to 18 carbon atoms, or a substituted or unsubstituted linked aromatic group in which two to three aromatic rings of these groups are linked. L¹ preferably represents a direct bond or a substituted or unsubstituted aromatic hydrocarbon group having 6 to 18 carbon atoms, and more preferably represents a direct bond. L² preferably represents a direct bond or a substituted or unsubstituted aromatic hydrocarbon group having 6 to 18 carbon atoms, and more preferably a substituted or unsubstituted aromatic hydrocarbon group having 6 to 12 carbon atoms. In L¹ and L², specific examples of the unsubstituted aromatic hydrocarbon group having 6 to 18 carbon atoms and the unsubstituted aromatic heterocyclic group having 3 to 18 carbon atoms are the same as described for Ar¹ to Ar³ except for groups obtained by removing two hydrogens from the aromatic hydrocarbon.

When L¹ and L² represent the linked aromatic group, the two carbazole (CBZ) groups described in the formulae (2) to (8) and the formula (1) may be bonded via any of the aromatic hydrocarbon group or the aromatic heterocyclic group in the linked aromatic group, or may be on both terminals or may be bonded in branch.

“c” to “f” represent the number of substitutions, “c”, “e”, and “f” each independently represent an integer of 0 to 3, and preferably 0 to 1. “d” represents an integer of 0 to 2, and preferably 0.

Ar⁴ to Ar¹² are each independently the same as described for Ar¹ to Ar³, provided that Ar⁴ to Ar¹², when representing the linked aromatic group, represent a substituted or unsubstituted linked aromatic group in which two to three aromatic rings are linked. Ar⁴ and Ar⁵ may be bonded to each other to form a cyclic structure.

When Ar¹ to Ar¹² represent the alkylarylamino group having 7 to 28 carbon atoms, the diarylamino group having 12 to 36 carbon atoms, the arylheteroarylamino group having 12 to 36 carbon atoms, the diheteroarylamino group having 12 to 36 carbon atoms, the aromatic hydrocarbon group, the aromatic heterocyclic group, or the linked aromatic group, these groups may have a substituent. Examples of this substituent include deuterium, an alkyl group having 1 to 20 carbon atoms, a cyano group, and an alkylsilyl group. The alkyl group having 1 to 20 carbon atoms may be any of linear, branched, and cyclic alkyl groups. This substituent is preferably deuterium, a linear, branched, or cyclic alkyl group having 1 to 10 carbon atoms, or a cyano group. Specific examples thereof include: linear saturated hydrocarbon groups, such as a methyl group, an ethyl group, a n-propyl group, a n-butyl group, a n-pentyl group, a n-hexyl group, a n-octyl group, a n-dodecyl group, a n-tetradecyl group, and a n-octadecyl group; branched saturated hydrocarbon groups, such as an isopropyl group, an isobutyl group, a neopentyl group, a 2-ethylhexyl group, and a 2-hexyloctyl group; and saturated alicyclic hydrocarbon groups, such as a cyclopentyl group, a cyclohexyl group, a cyclooctyl group, a 4-butylcyclohexyl group, and a 4-dodecylcyclohexyl group. In the present invention, the substituent is placed on the aromatic ring.

Preferable specific examples of the compound represented by the general formula (1) being the material for a photoelectric conversion device of the present invention are shown below, but the material is not limited thereto.

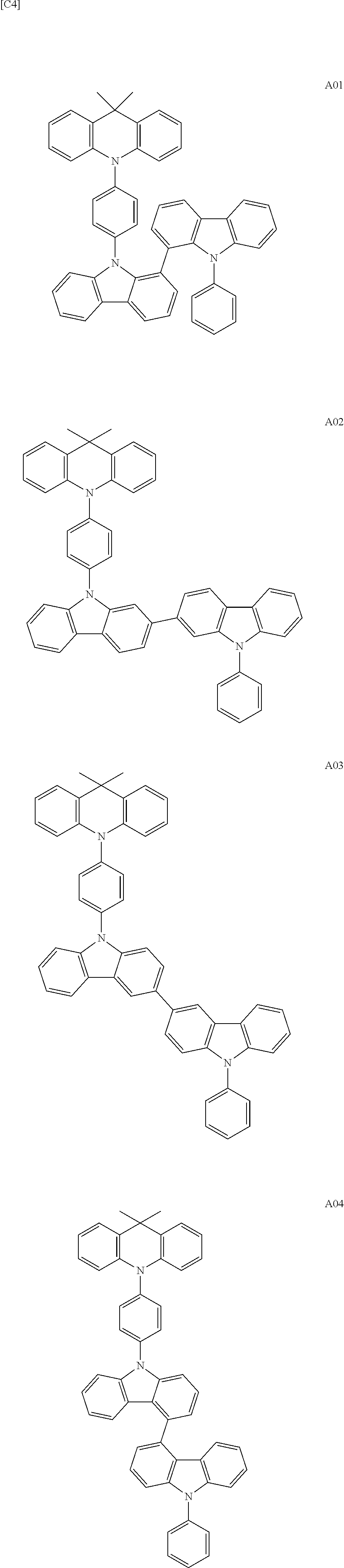

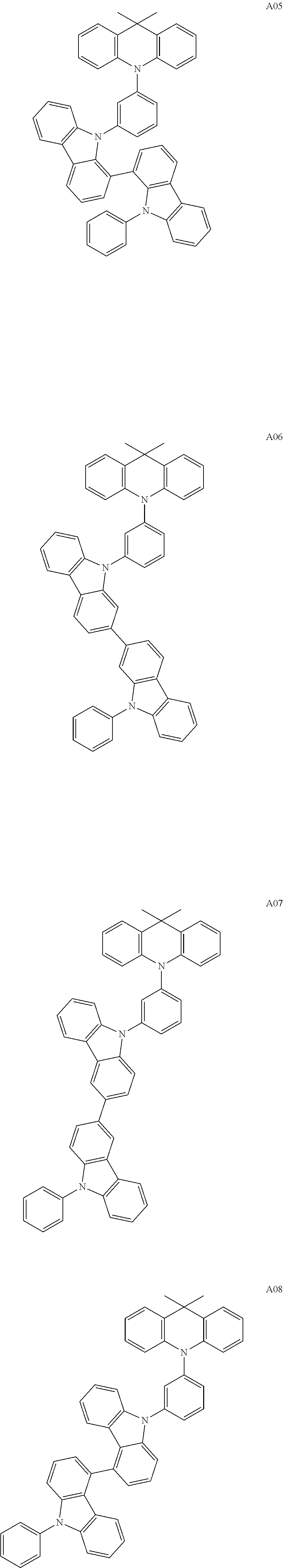

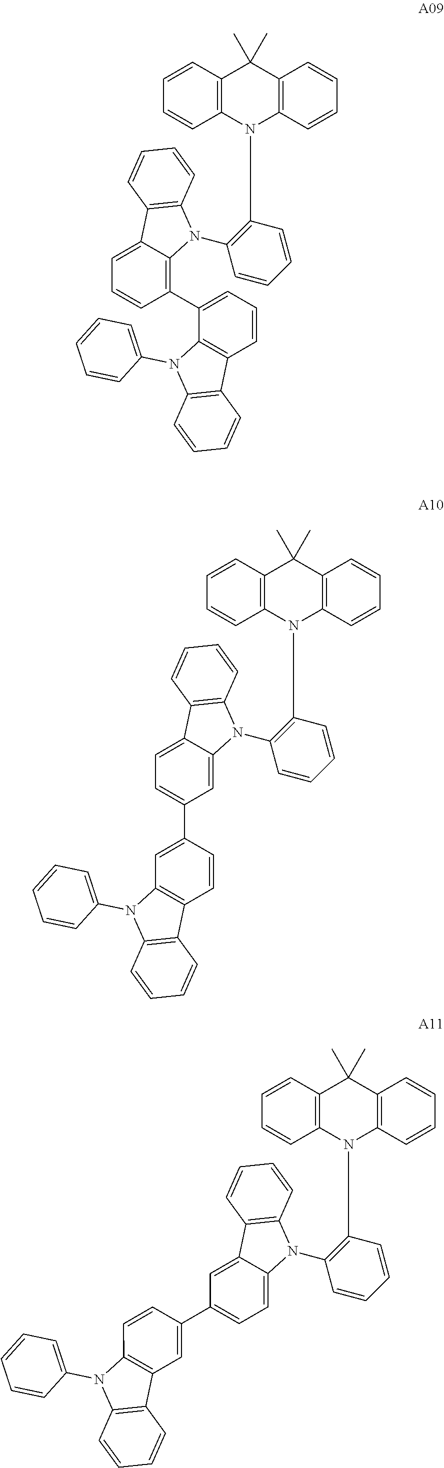

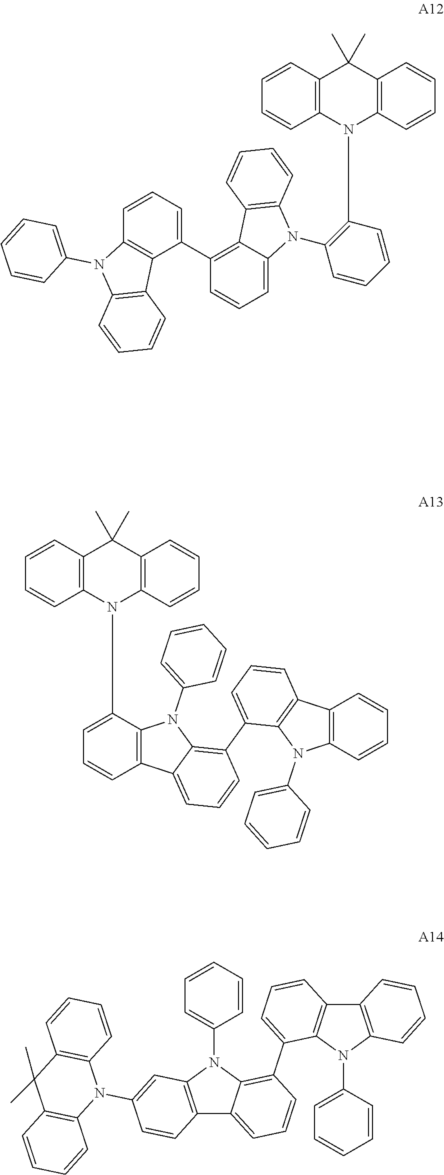

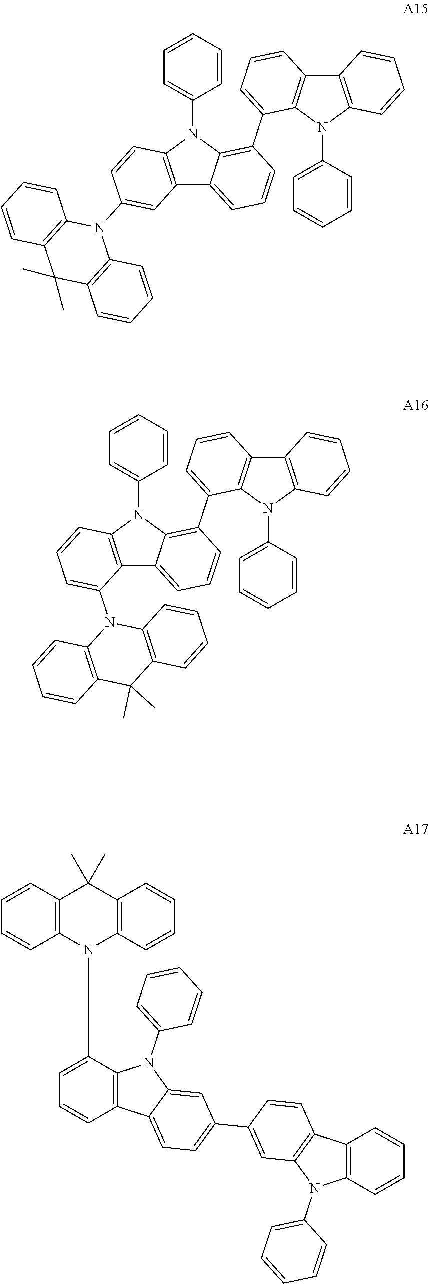

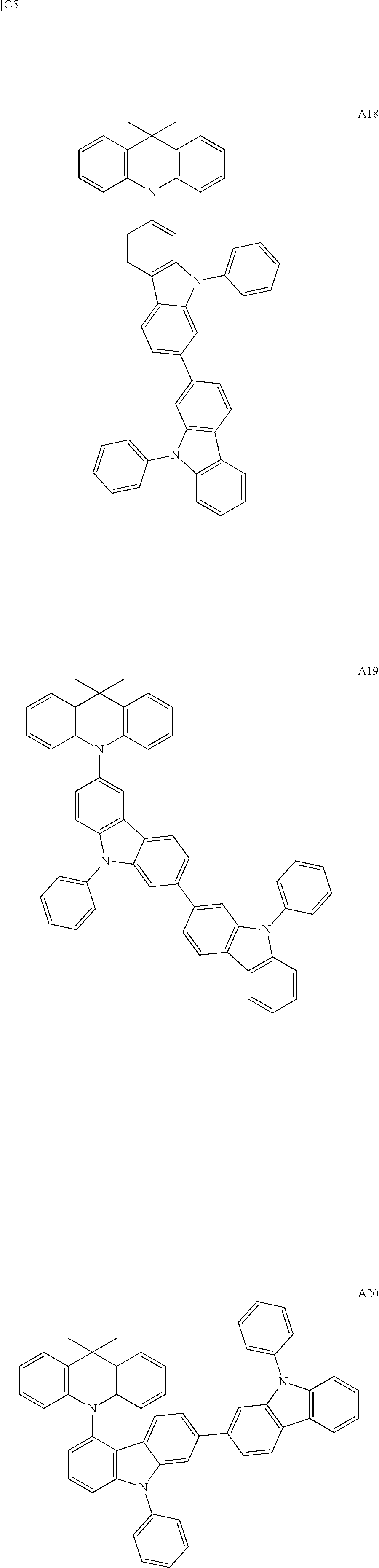

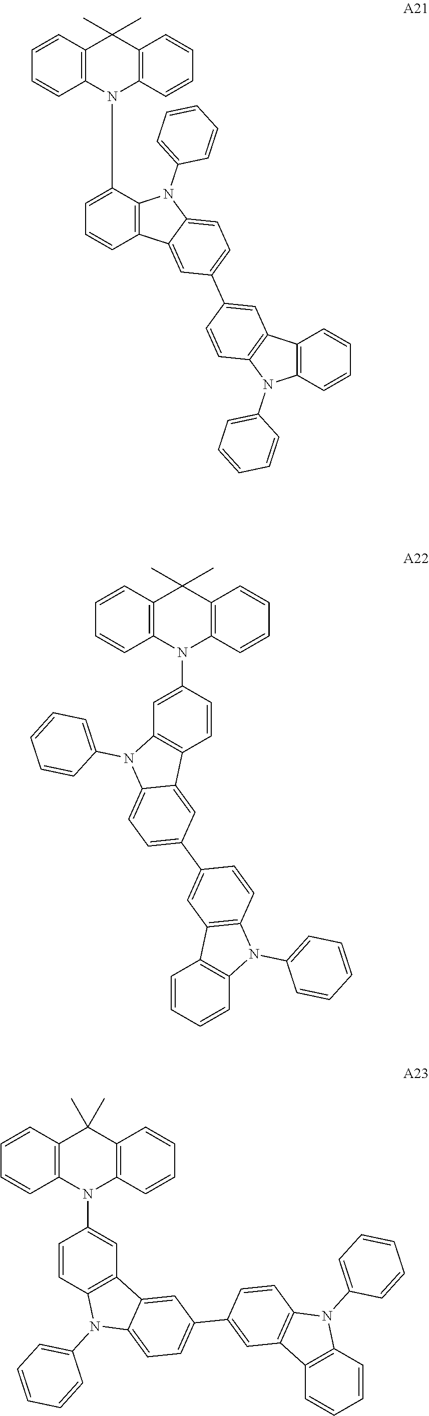

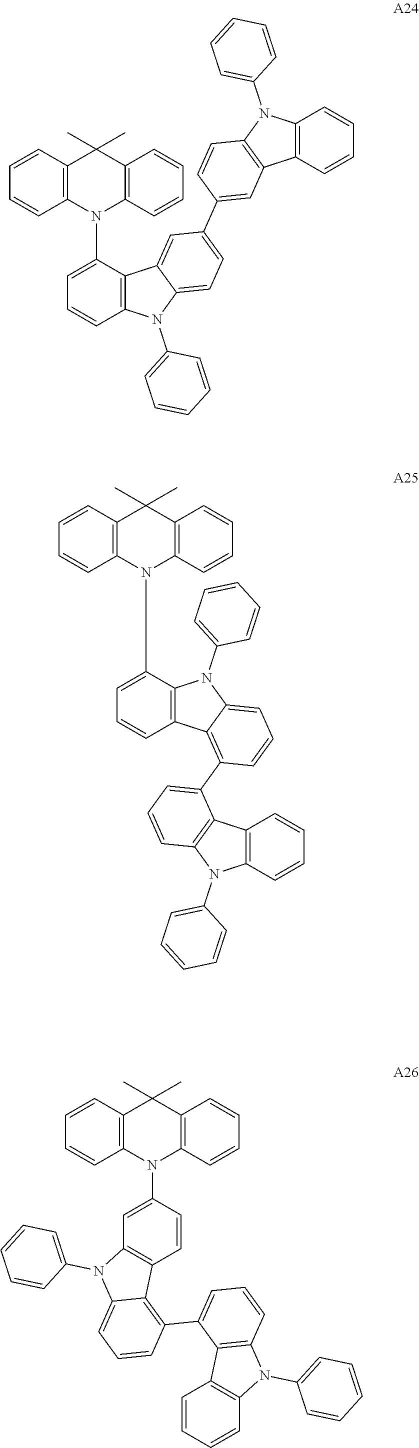

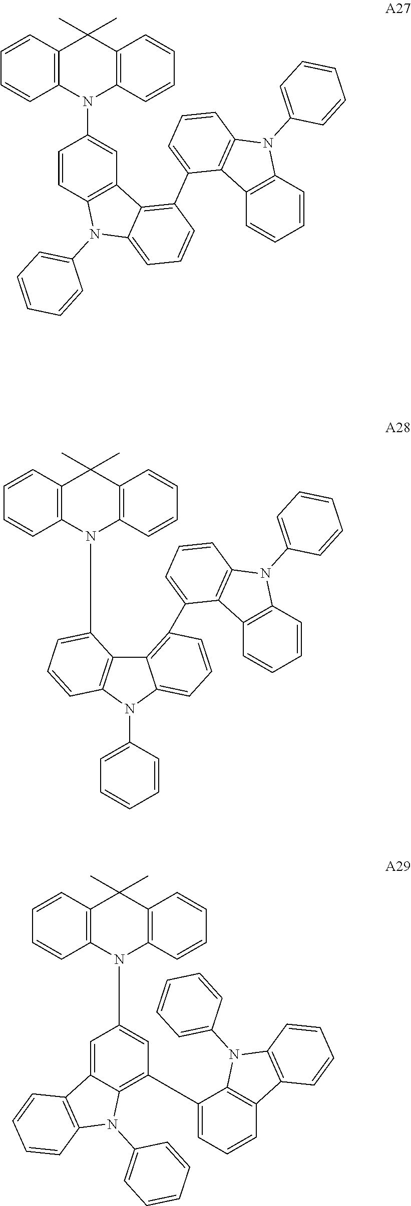

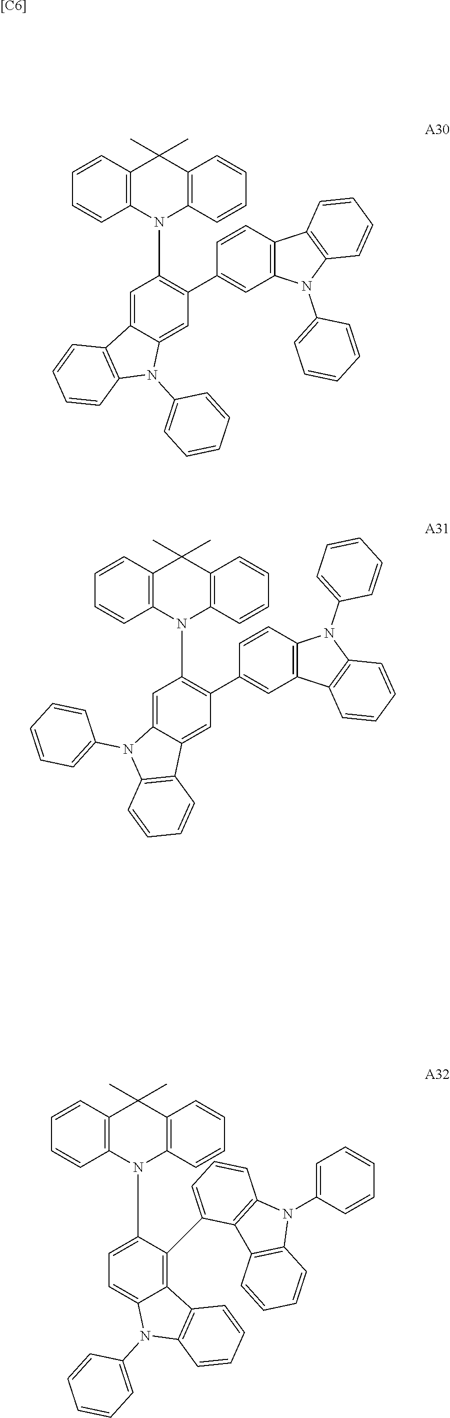

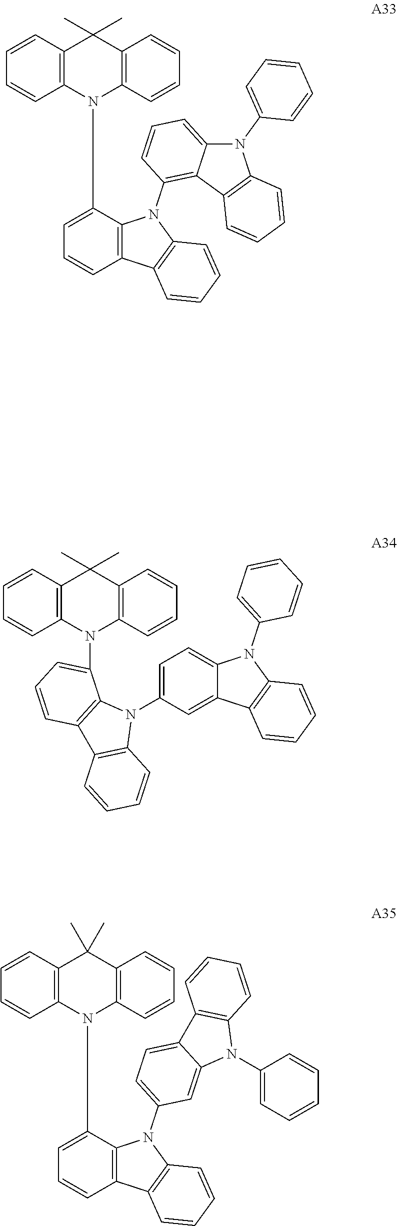

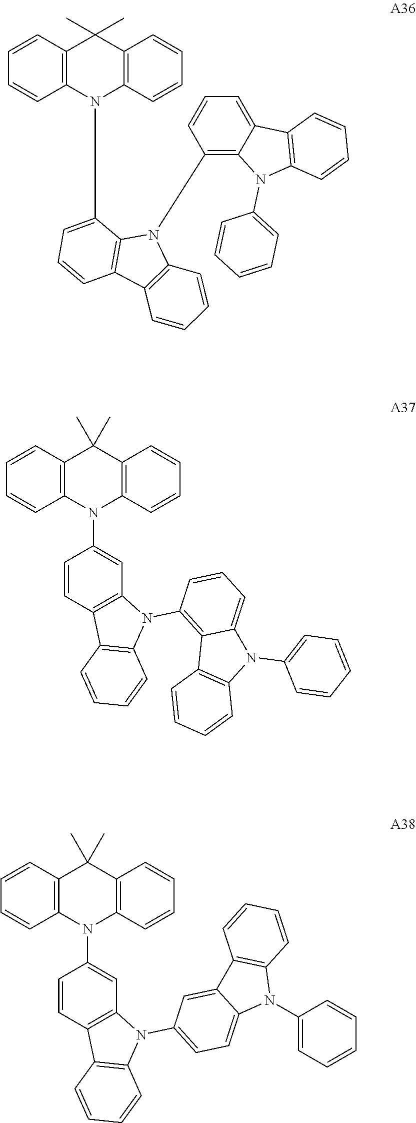

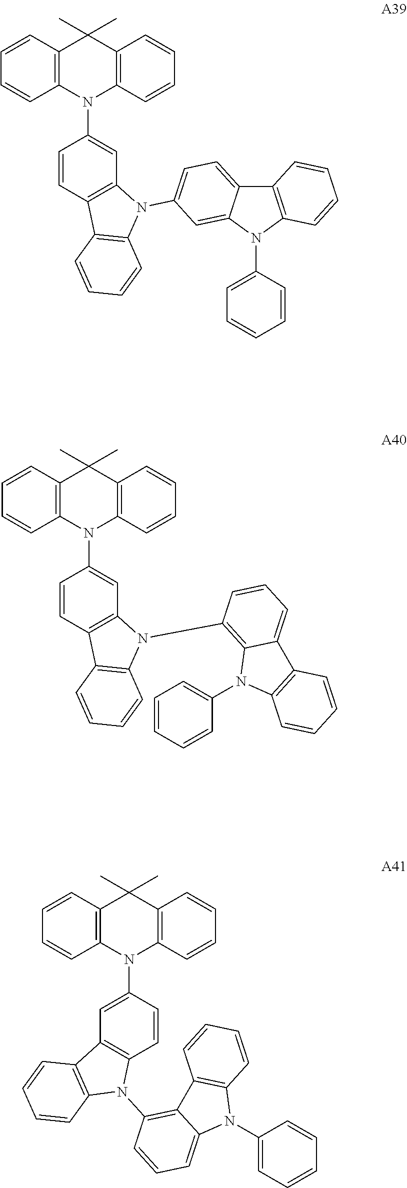

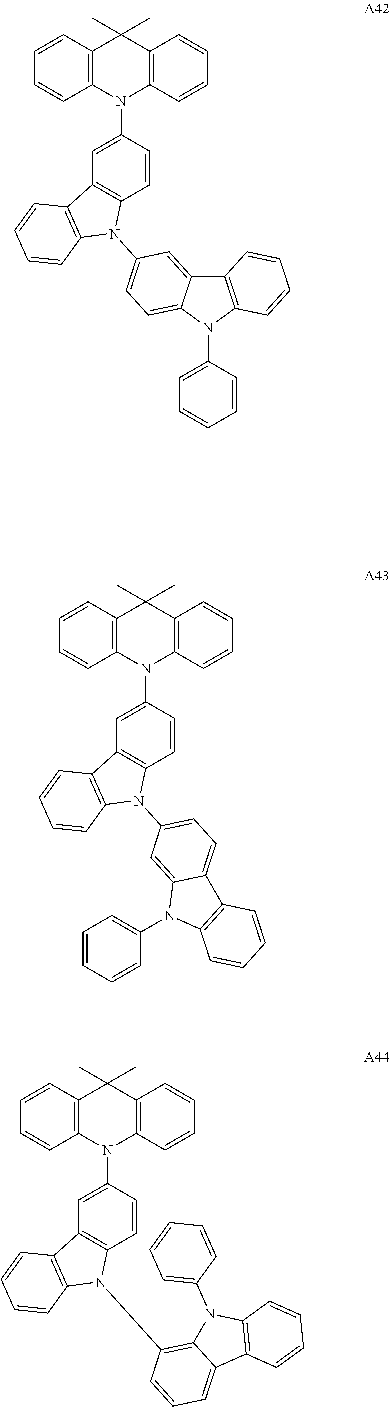

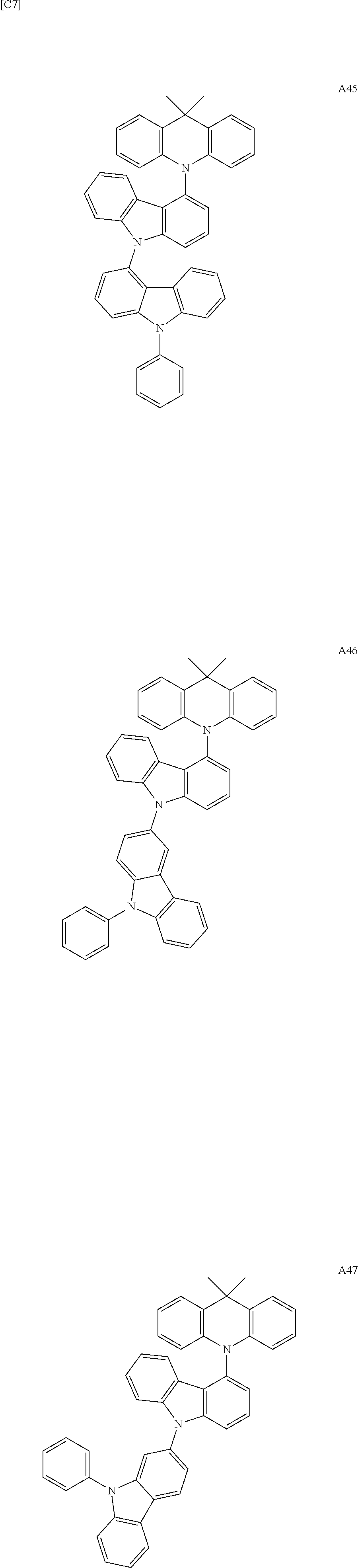

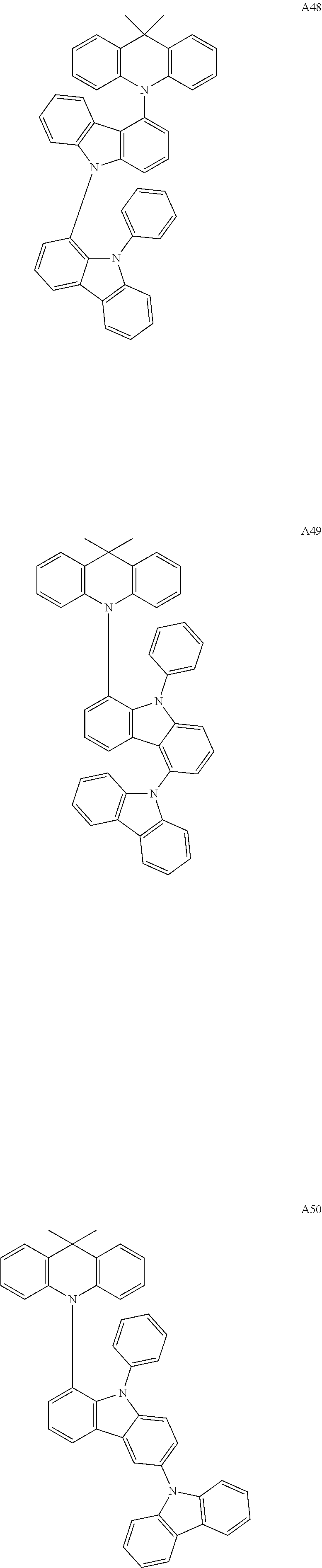

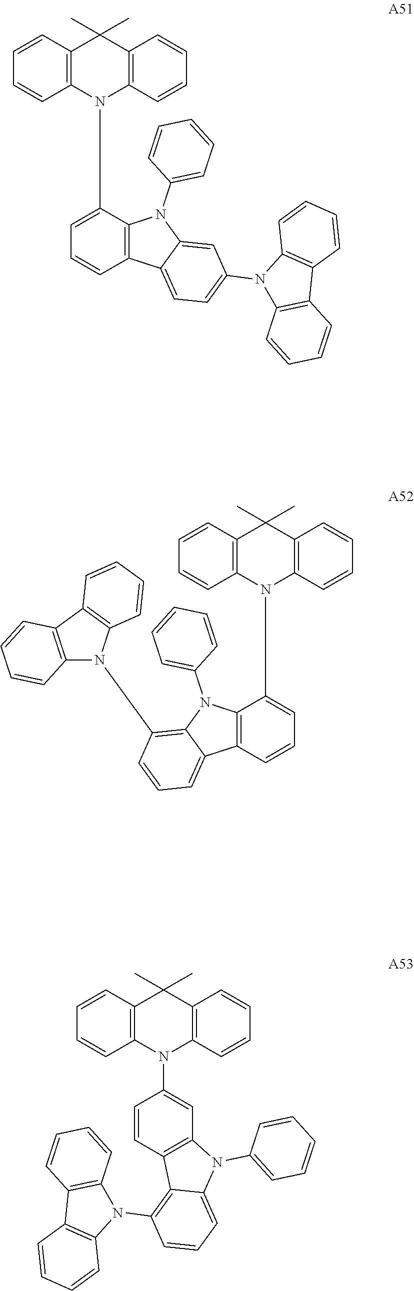

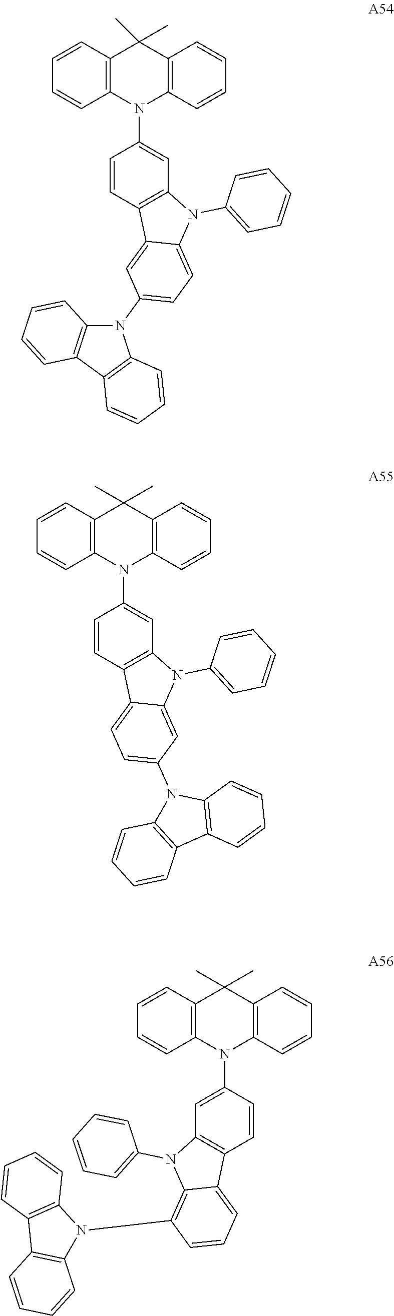

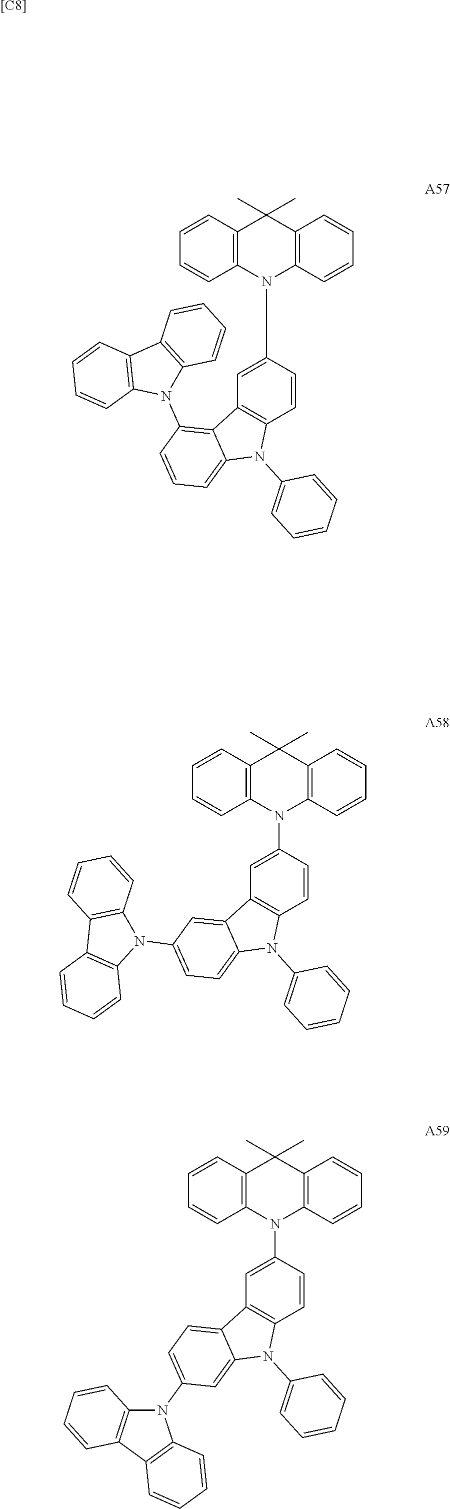

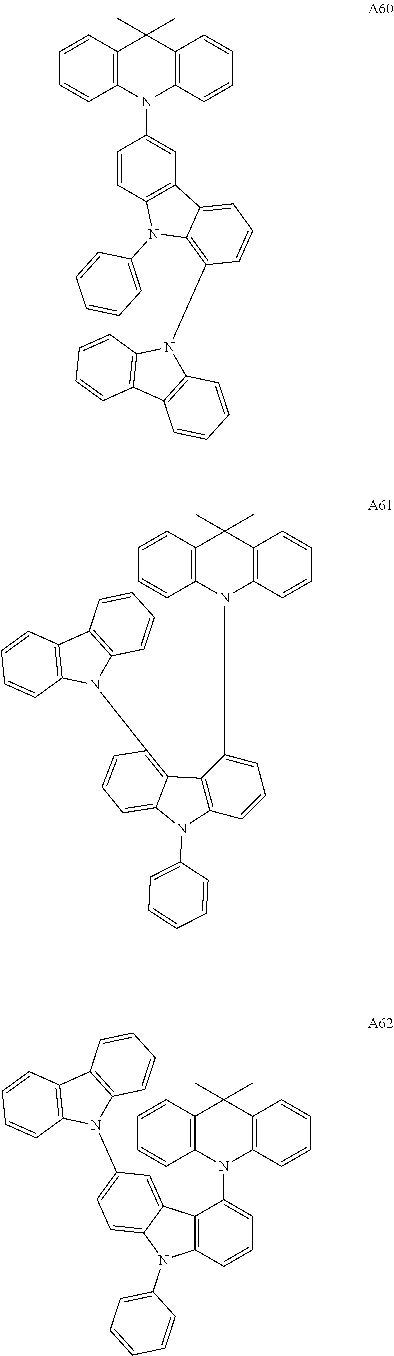

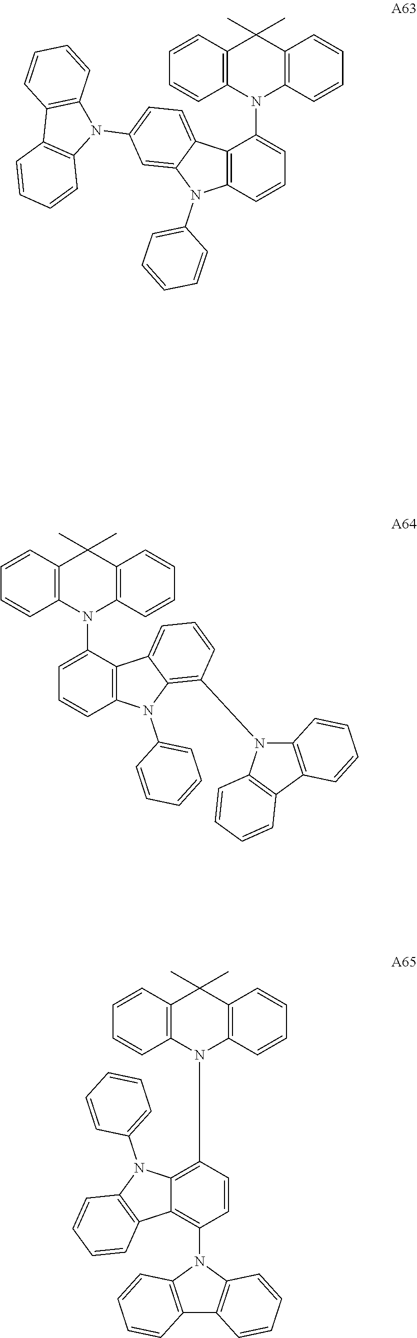

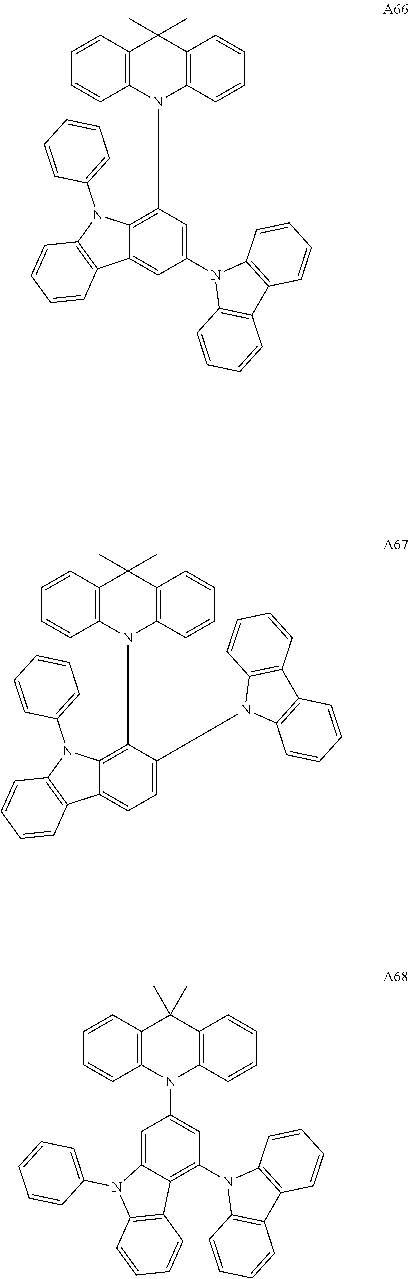

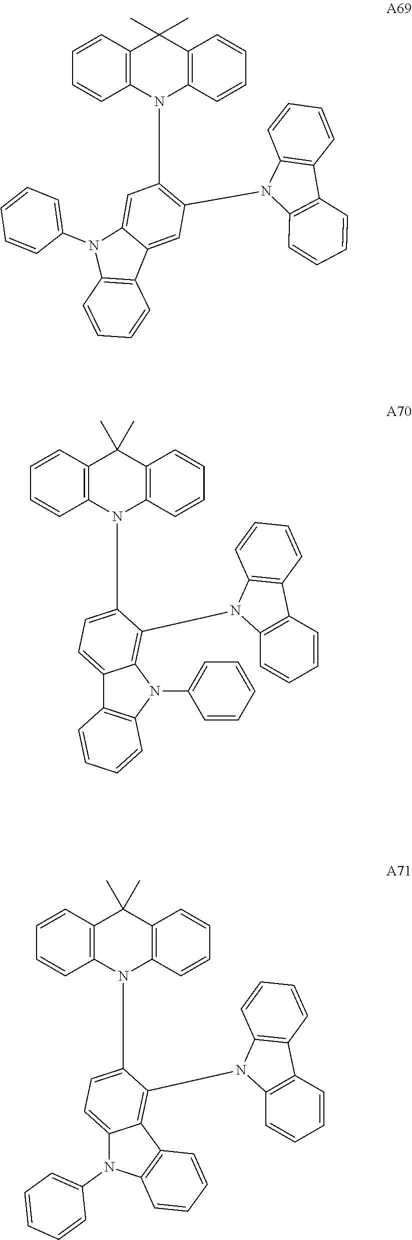

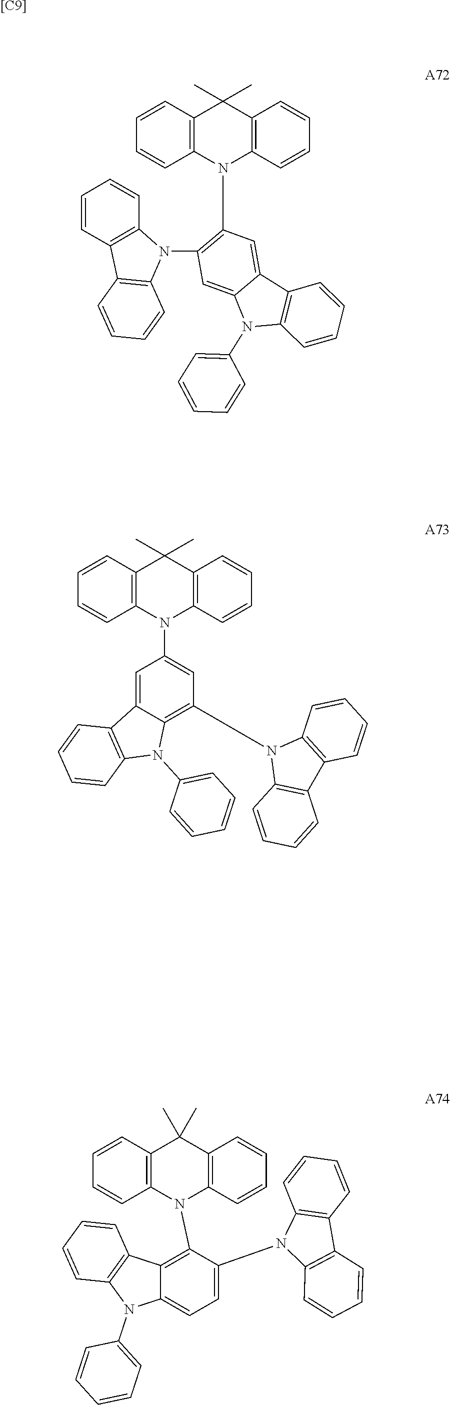

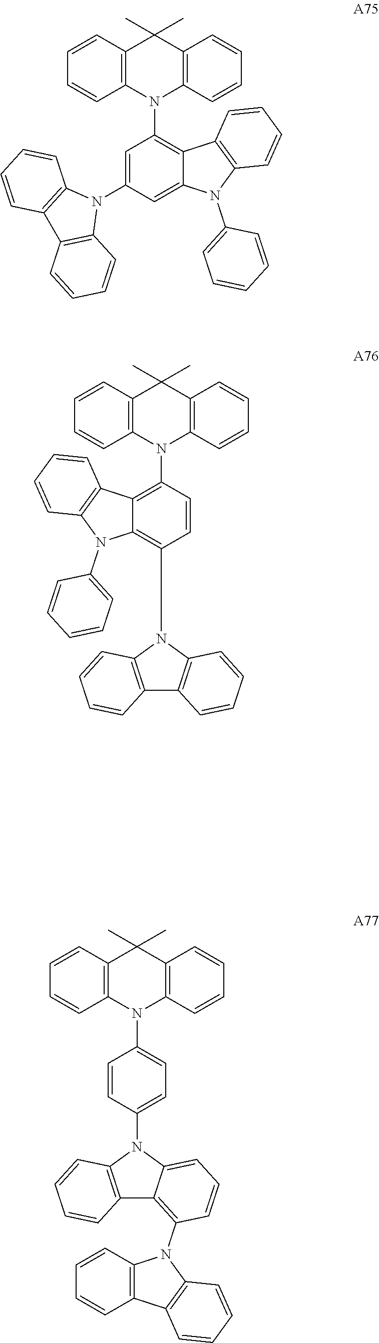

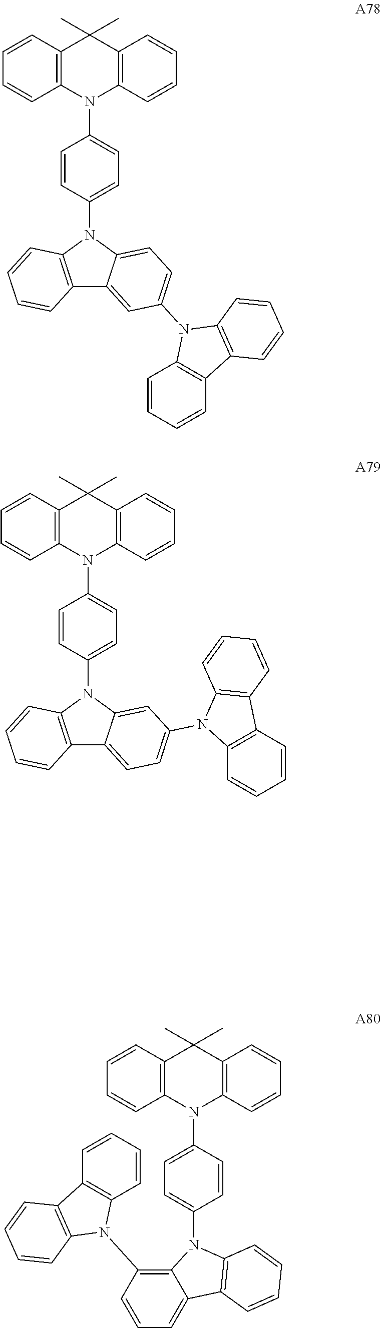

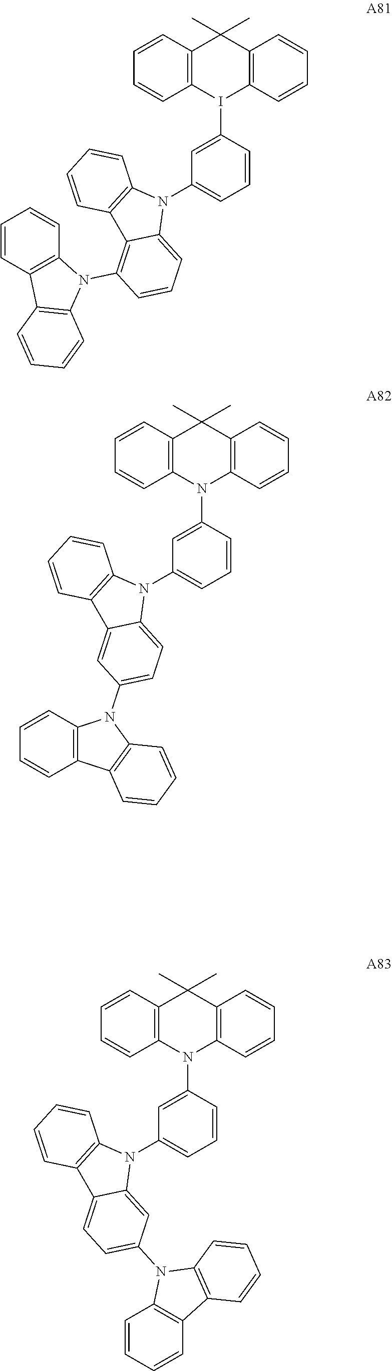

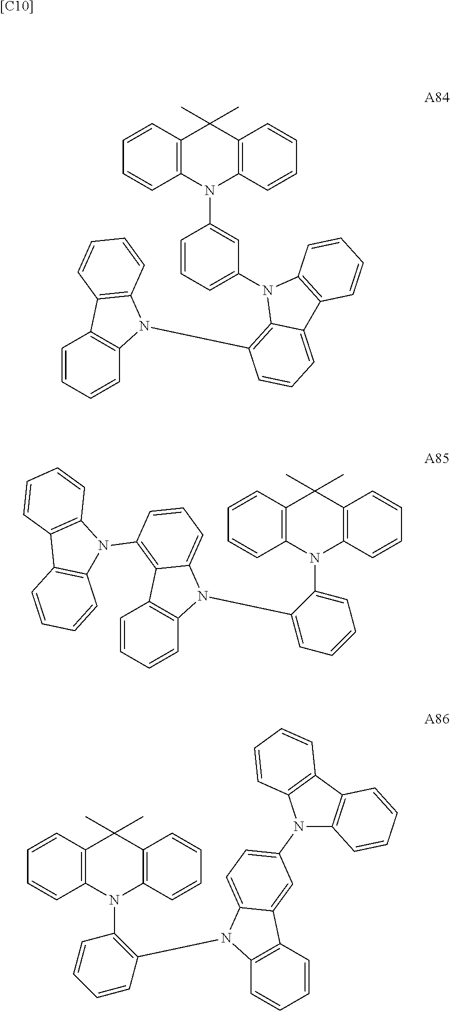

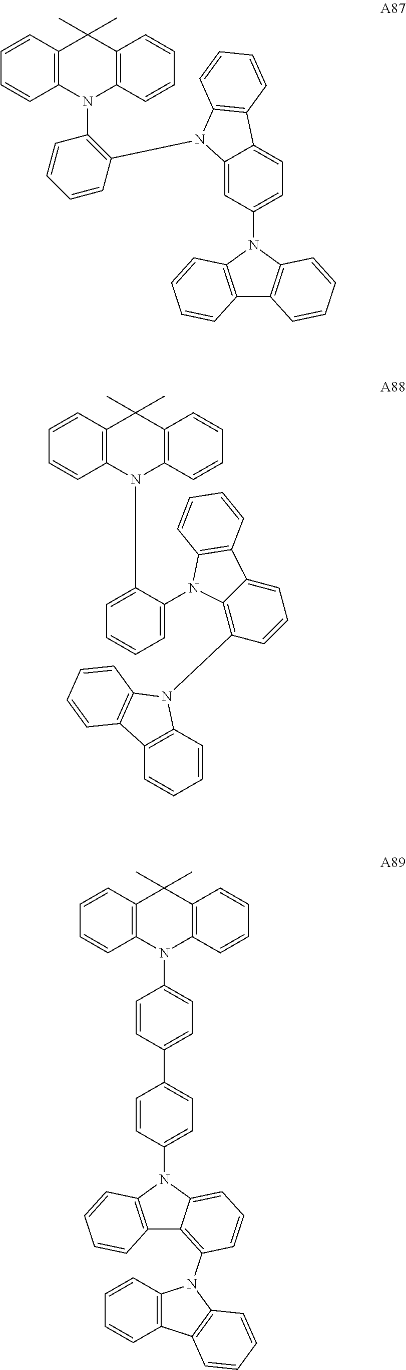

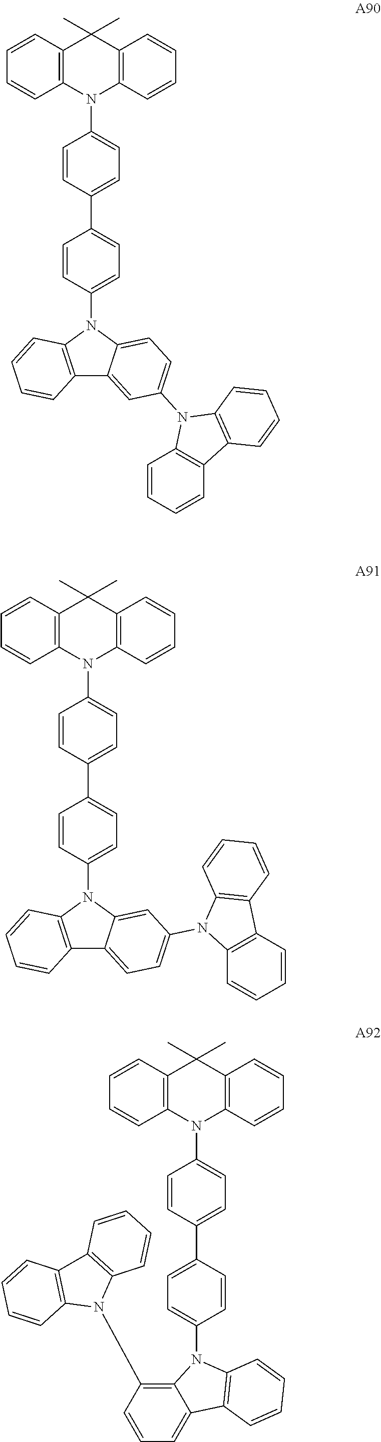

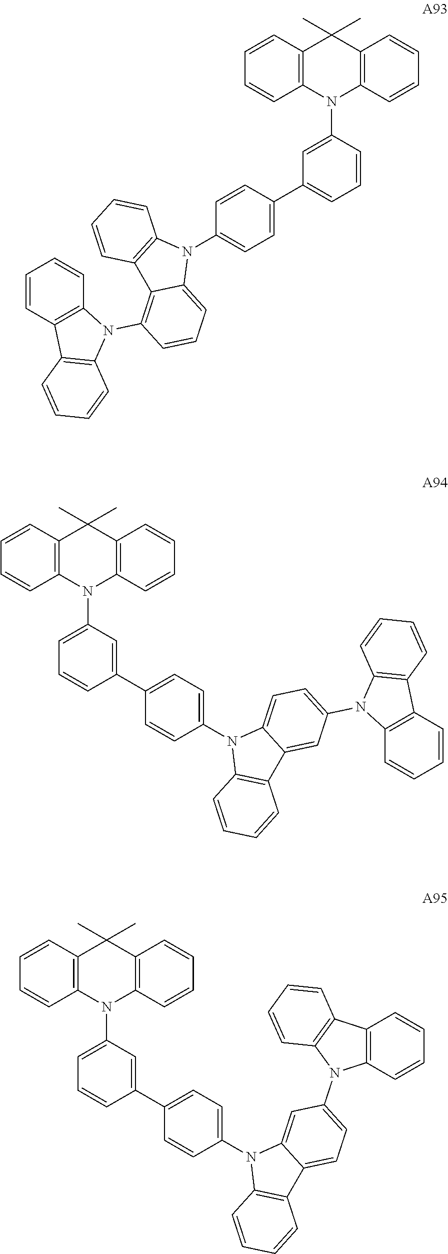

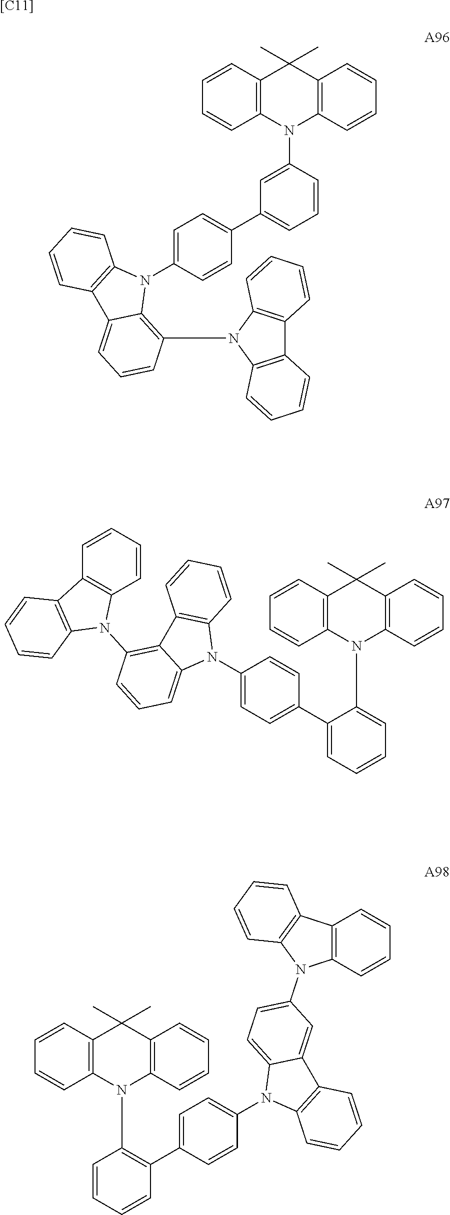

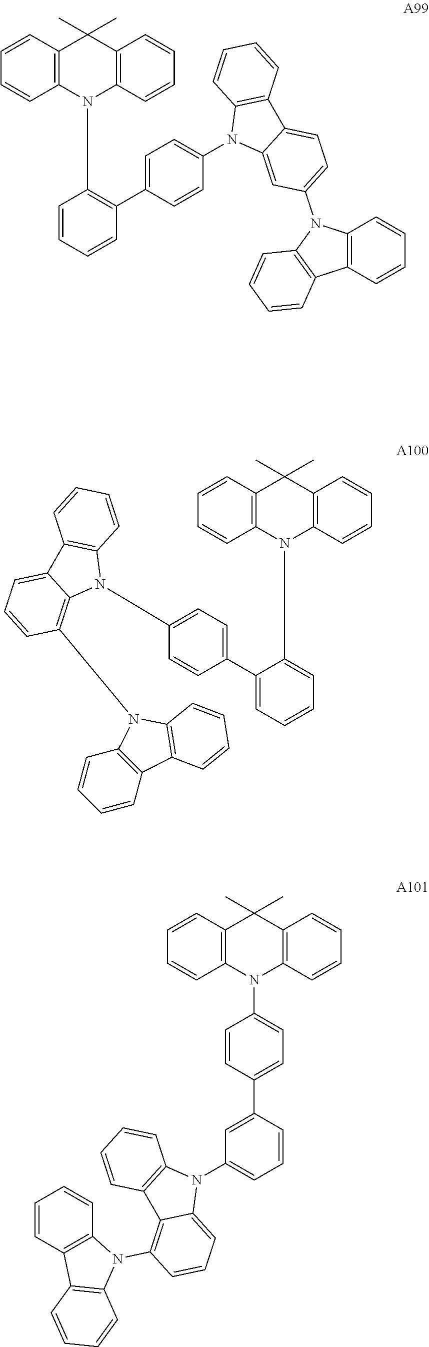

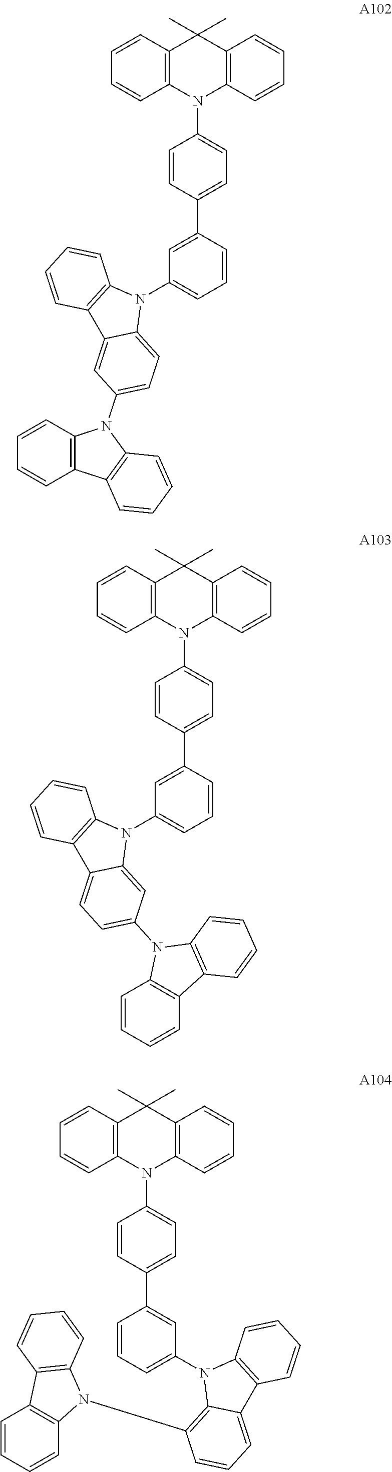

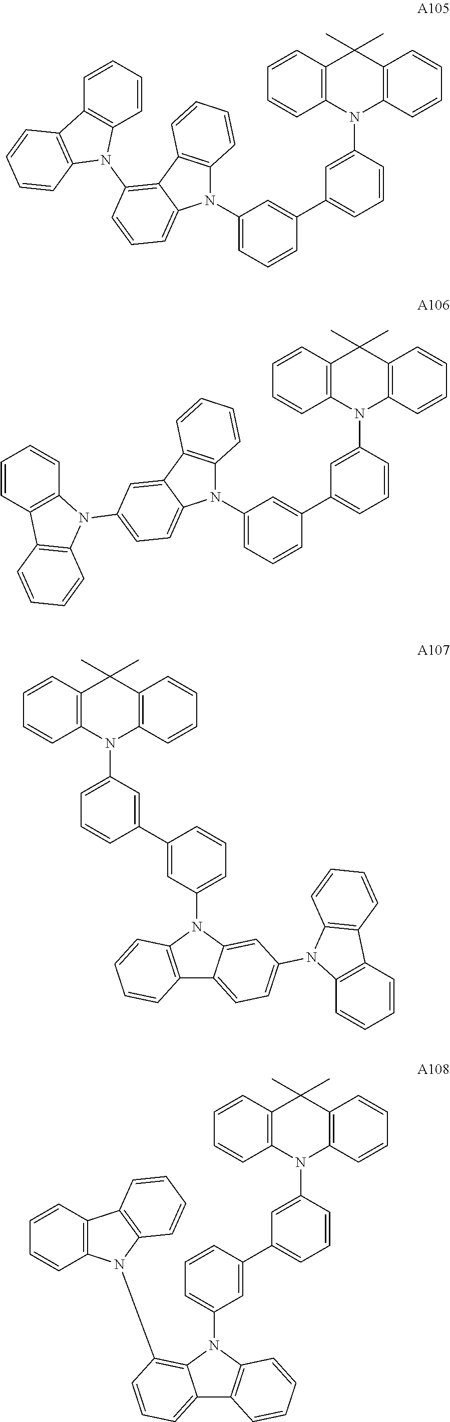

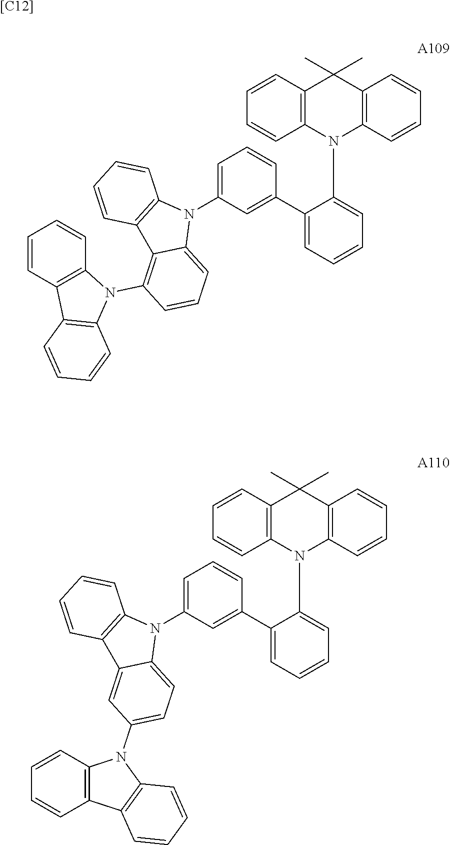

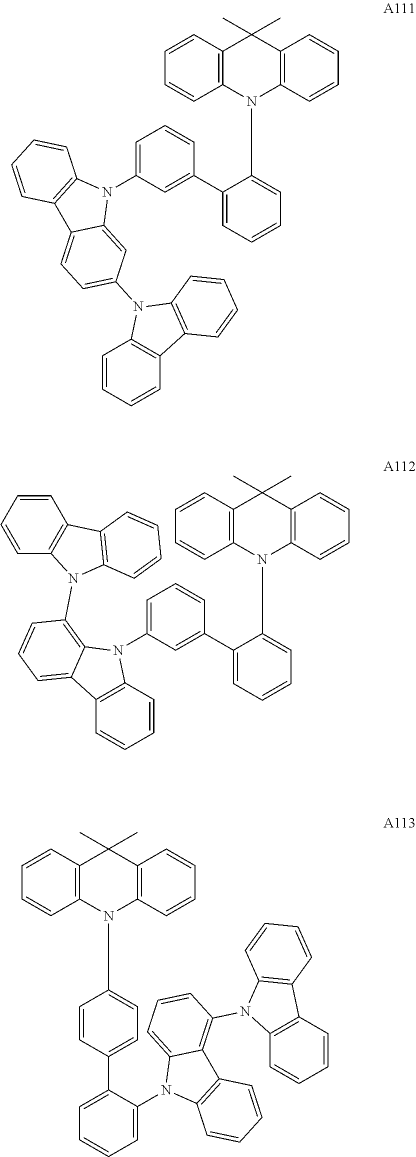

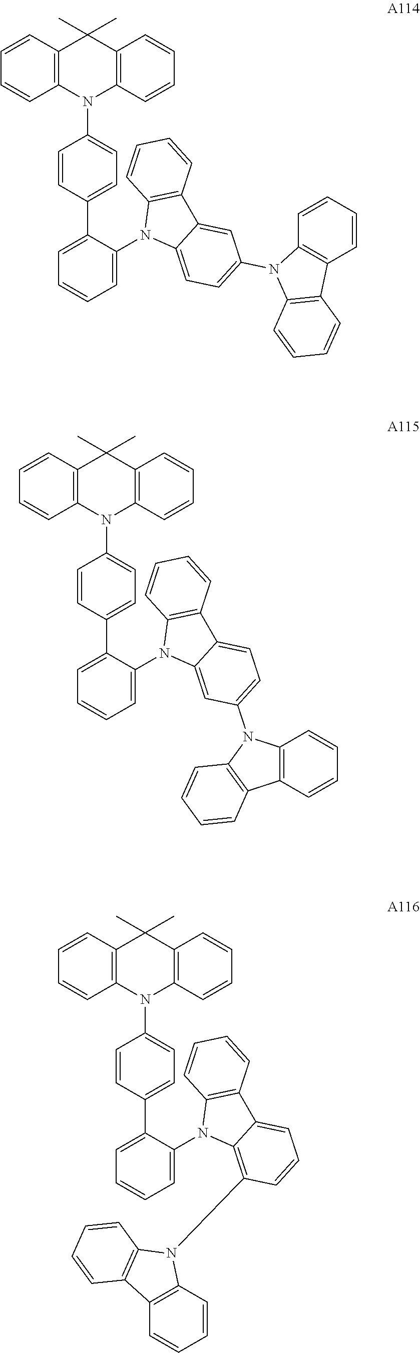

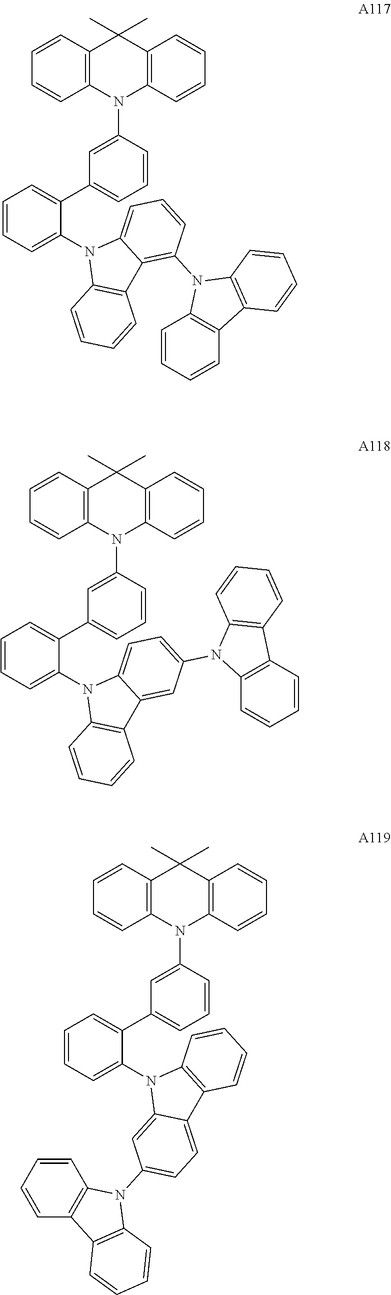

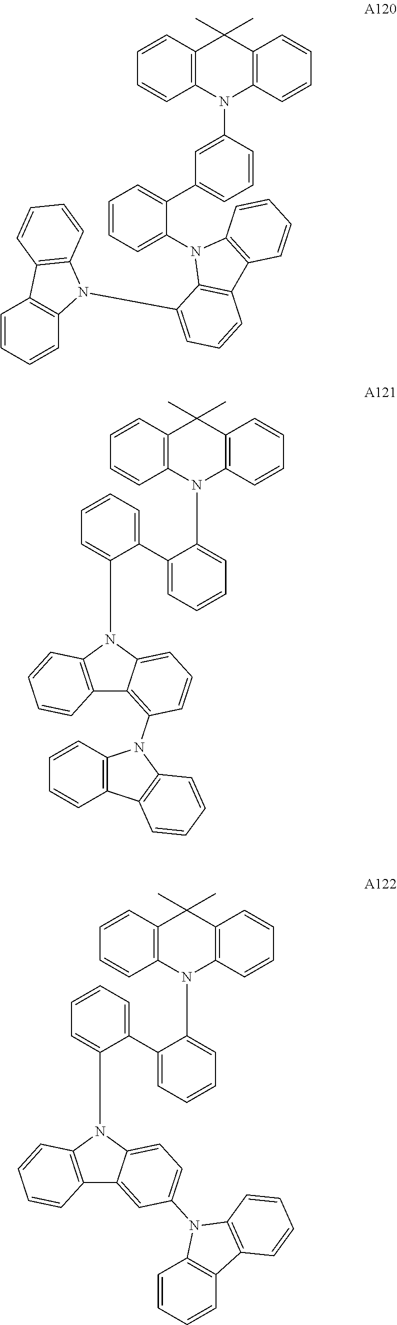

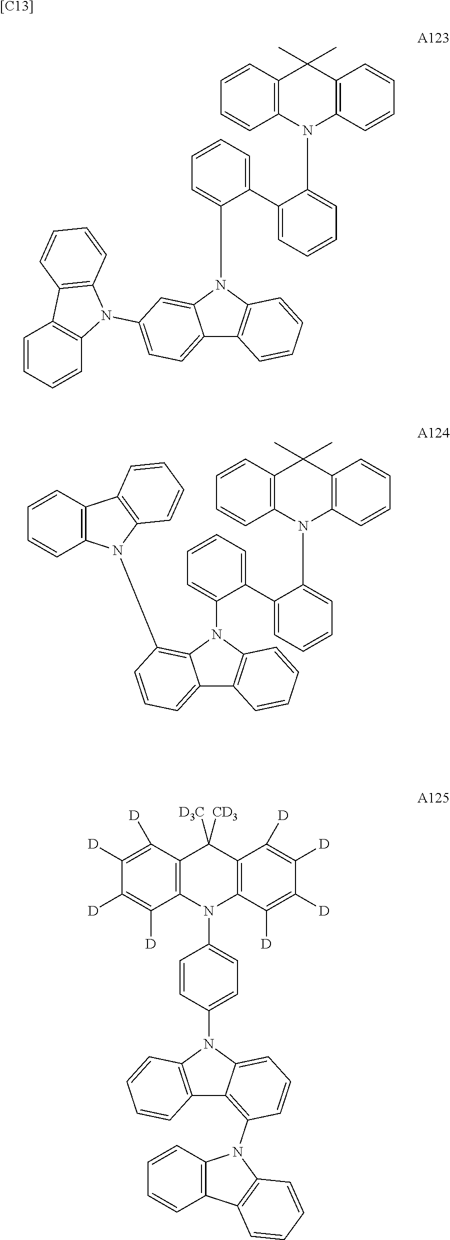

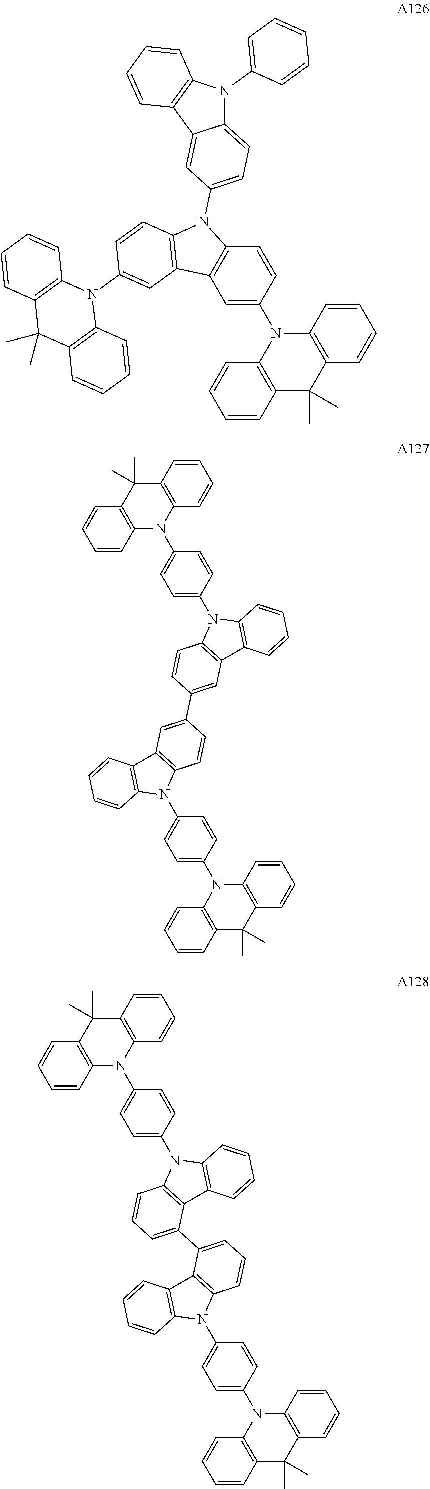

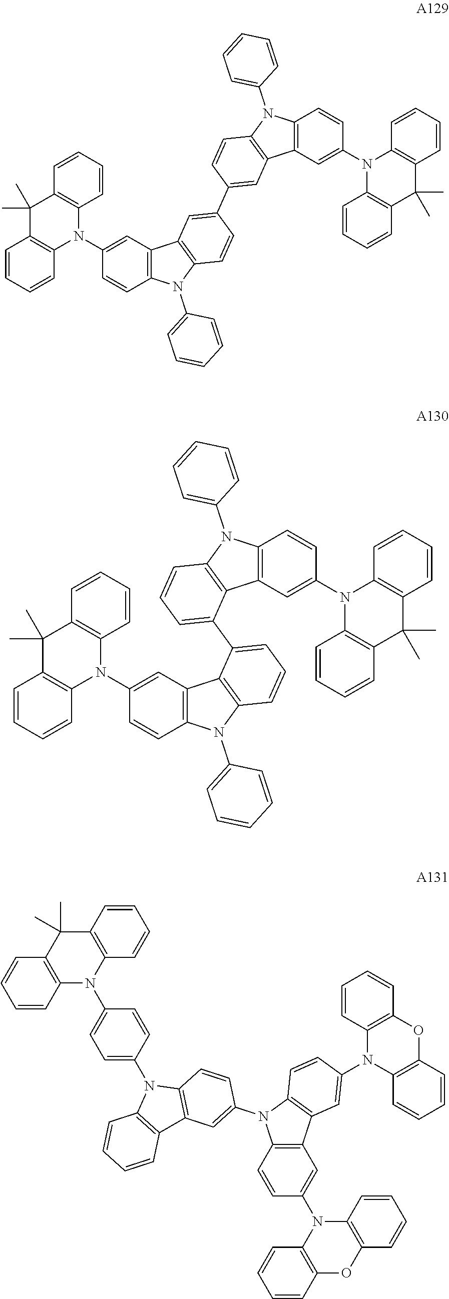

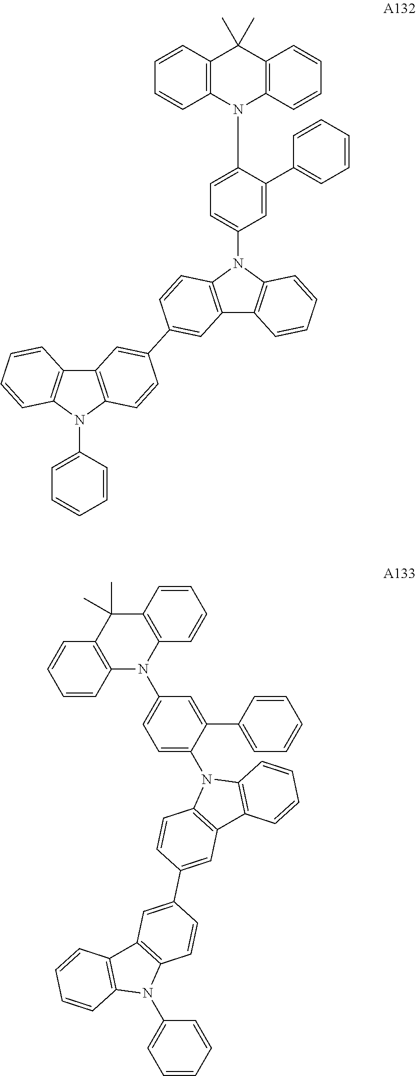

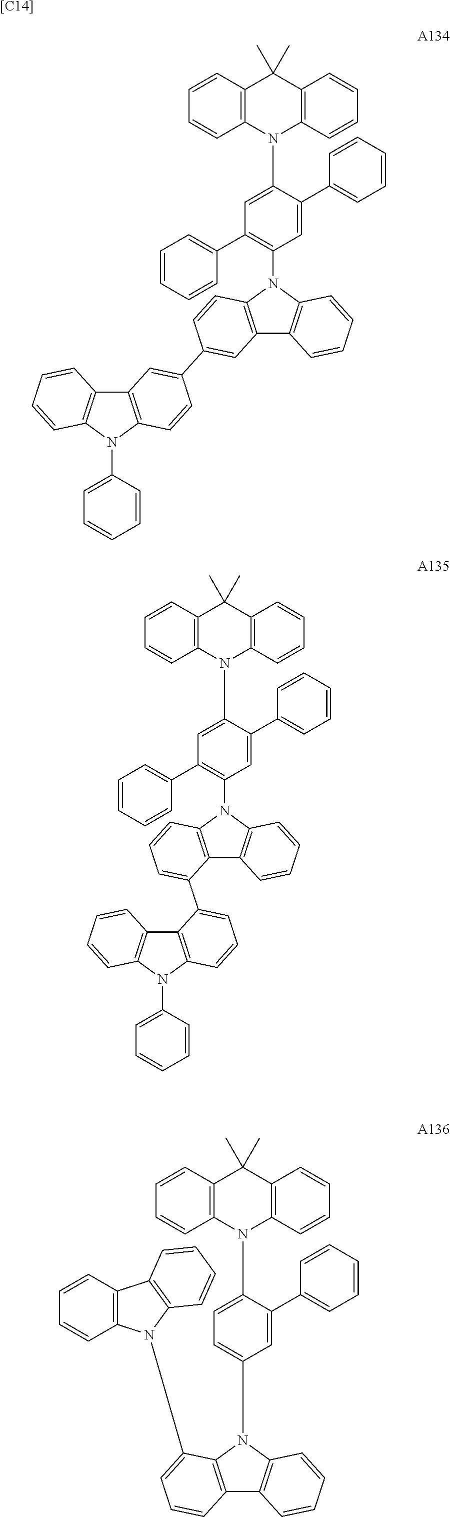

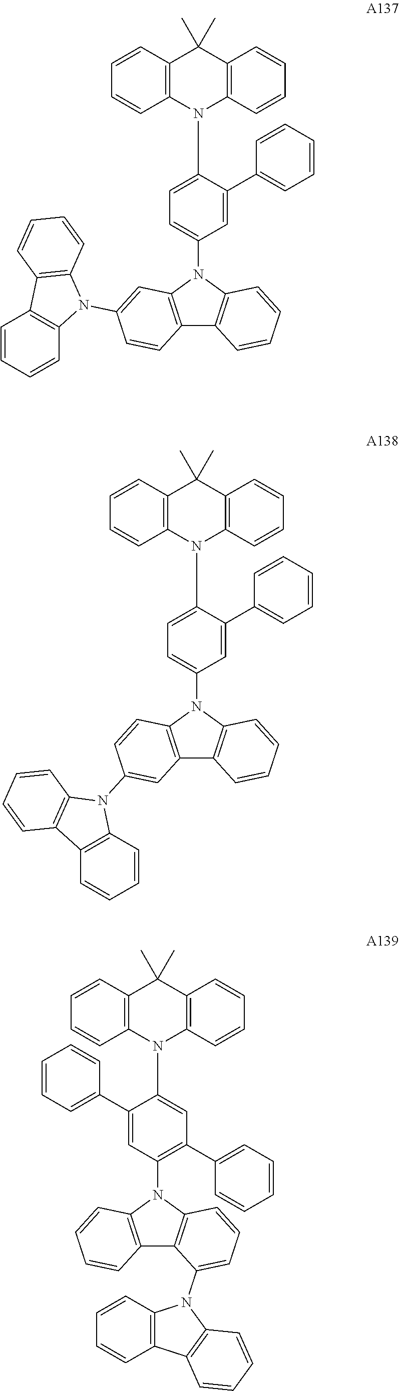

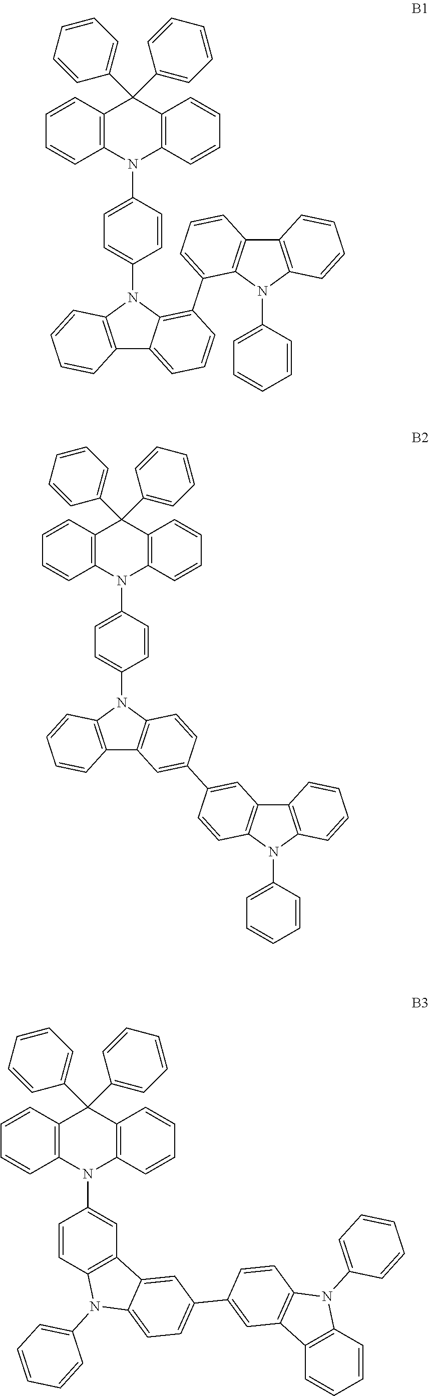

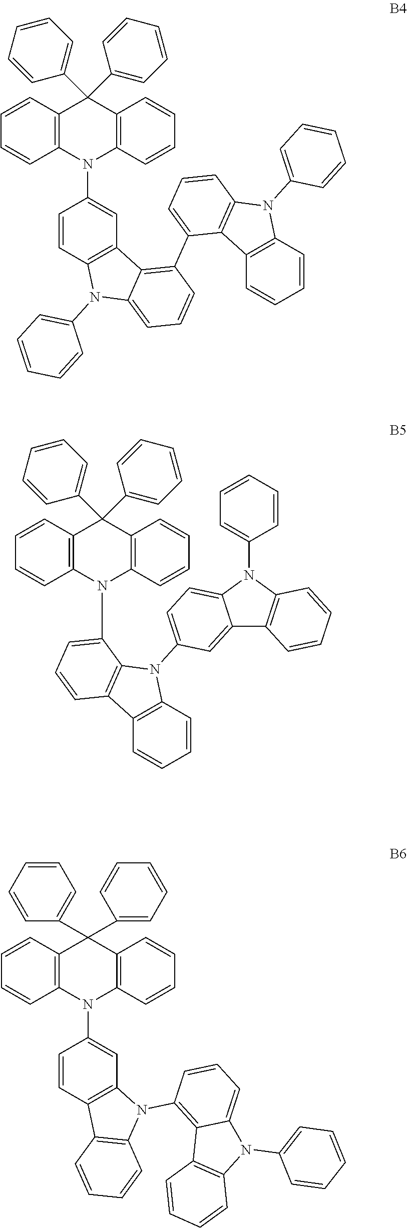

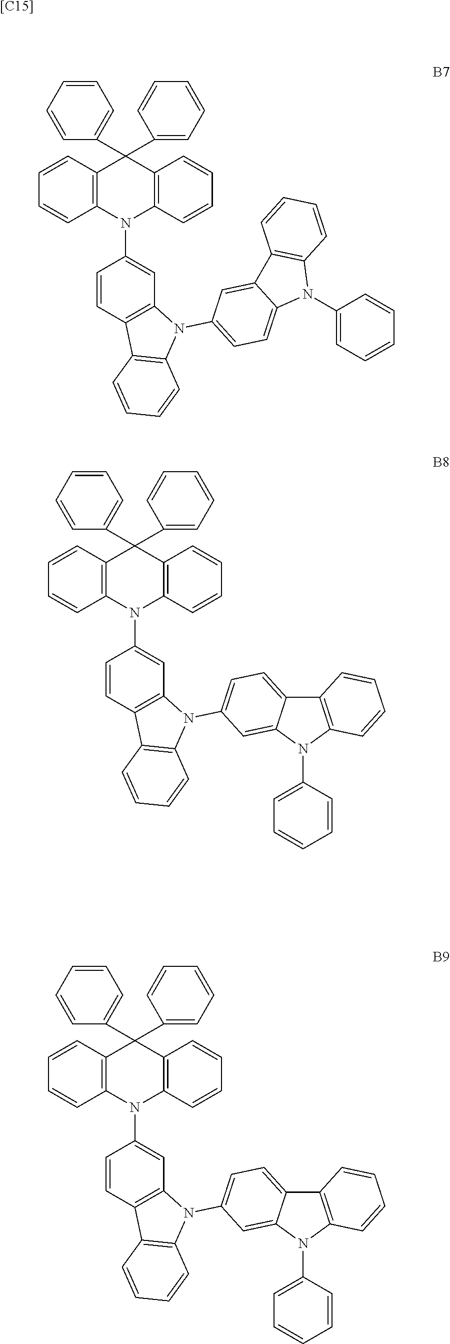

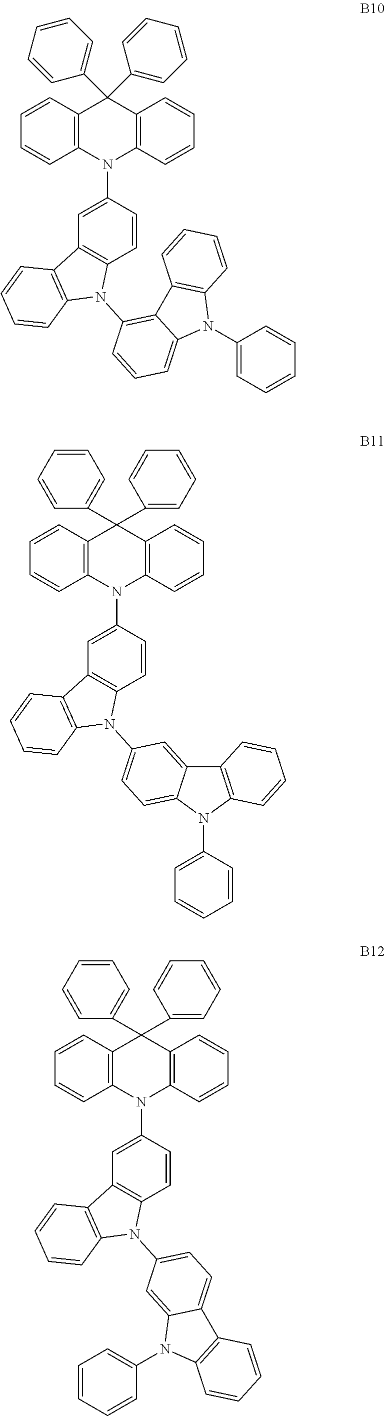

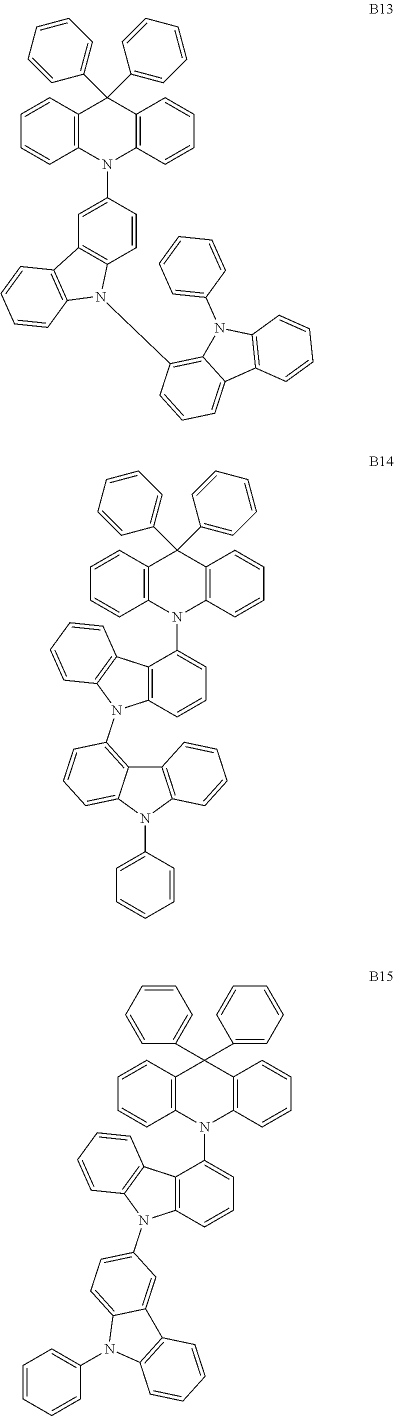

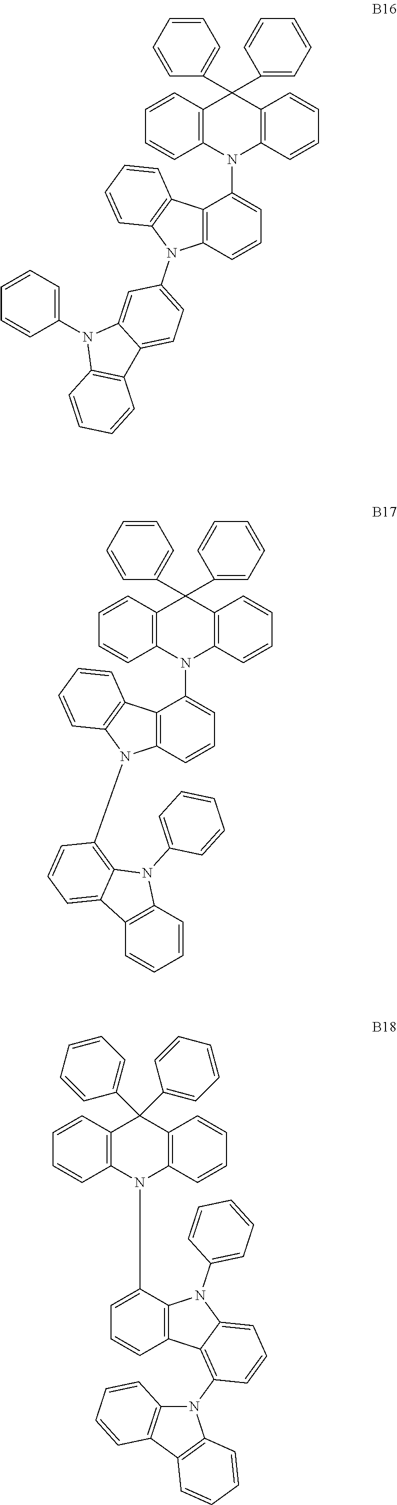

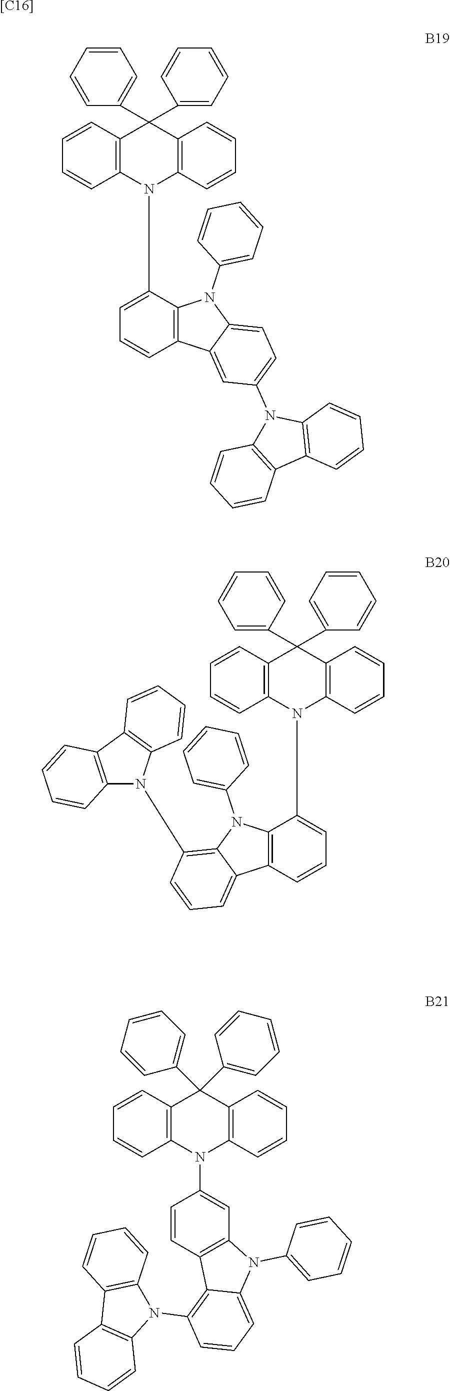

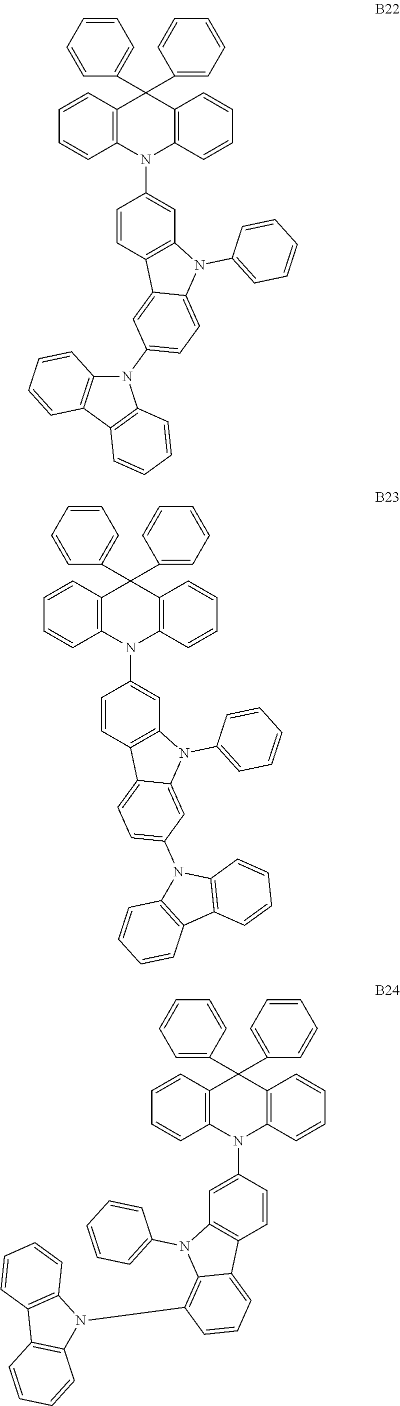

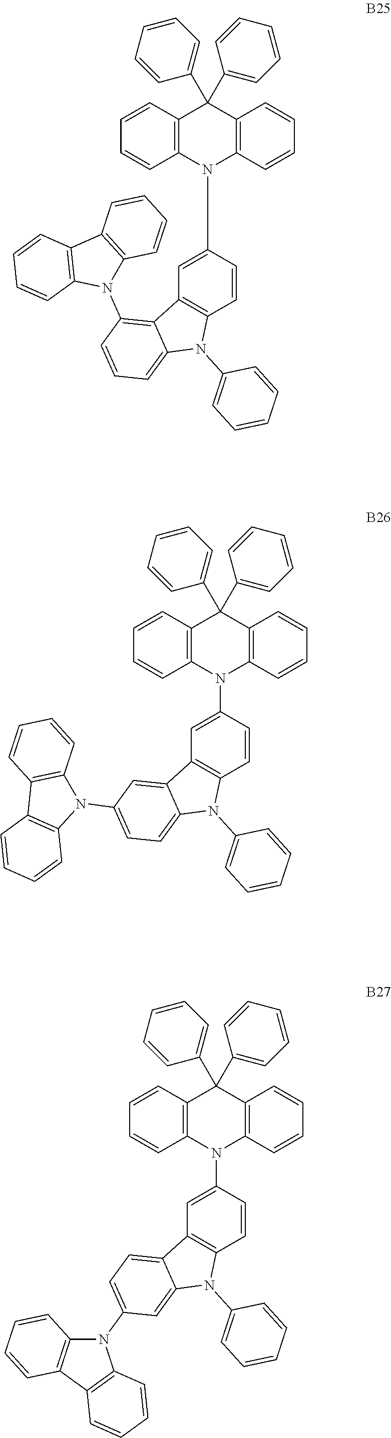

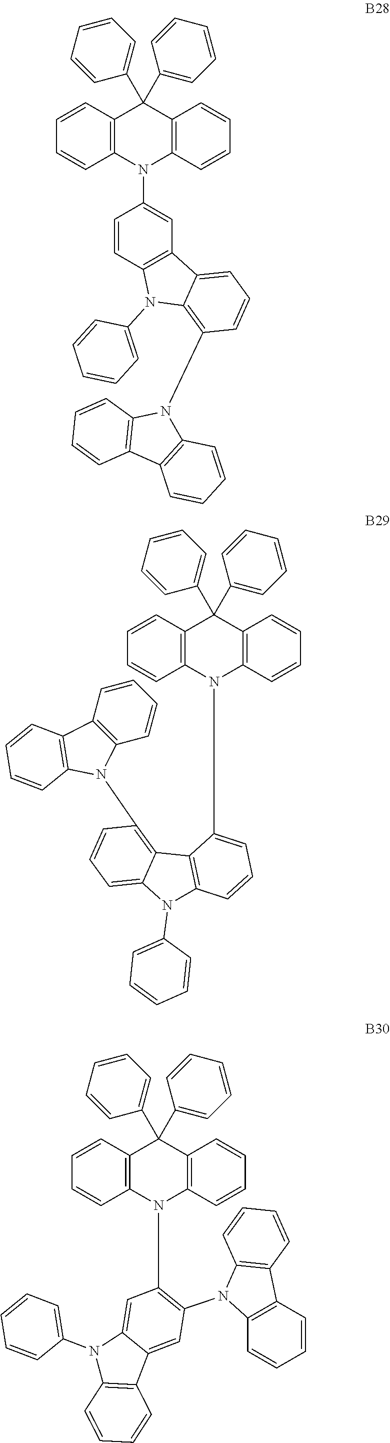

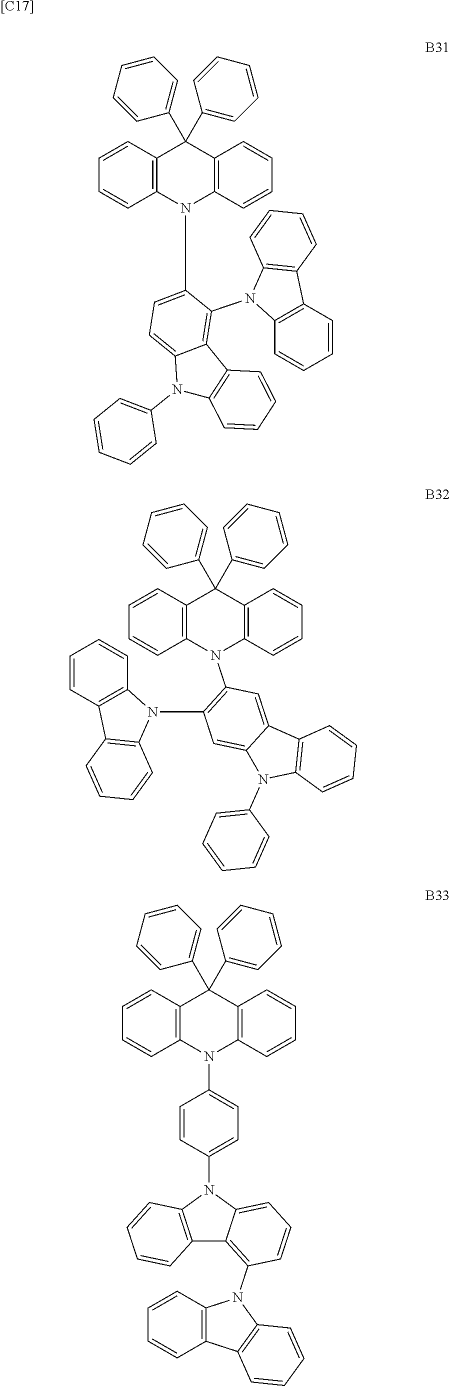

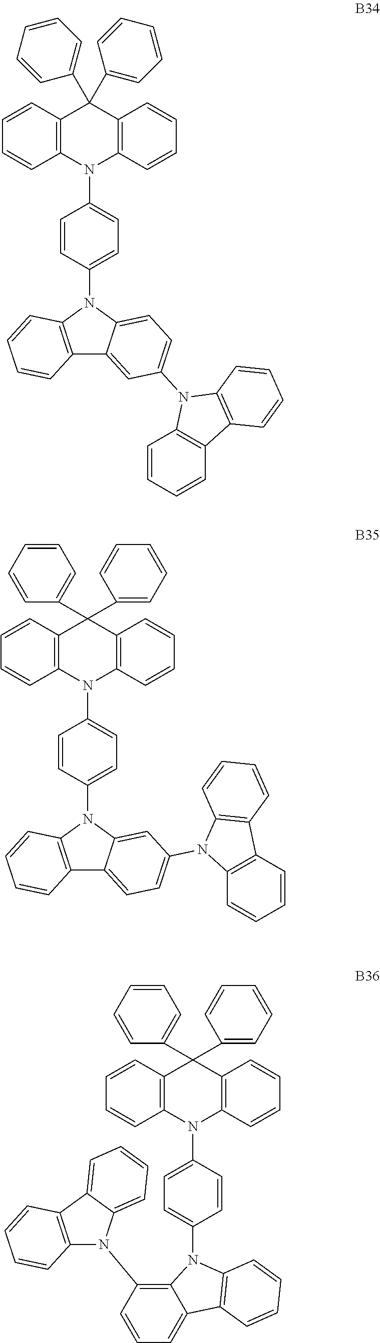

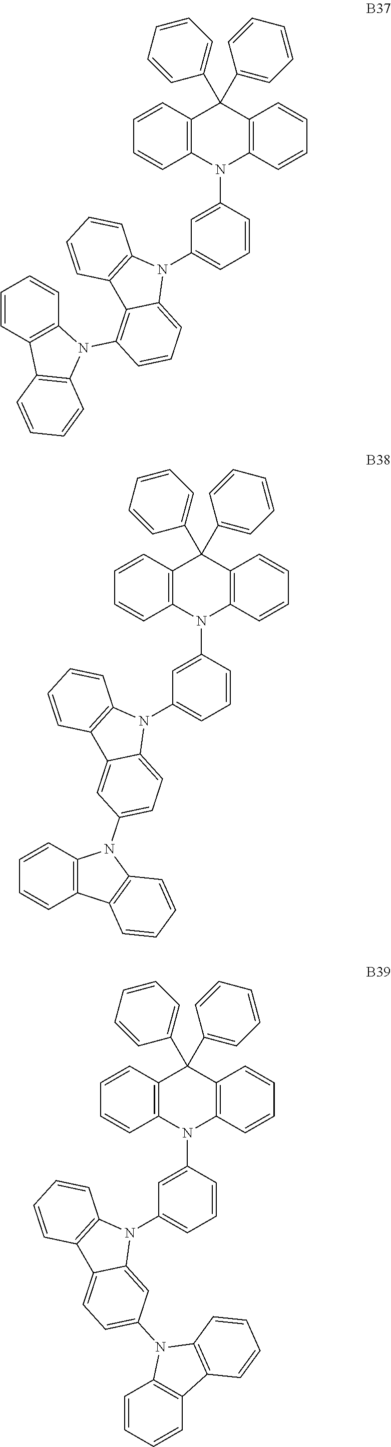

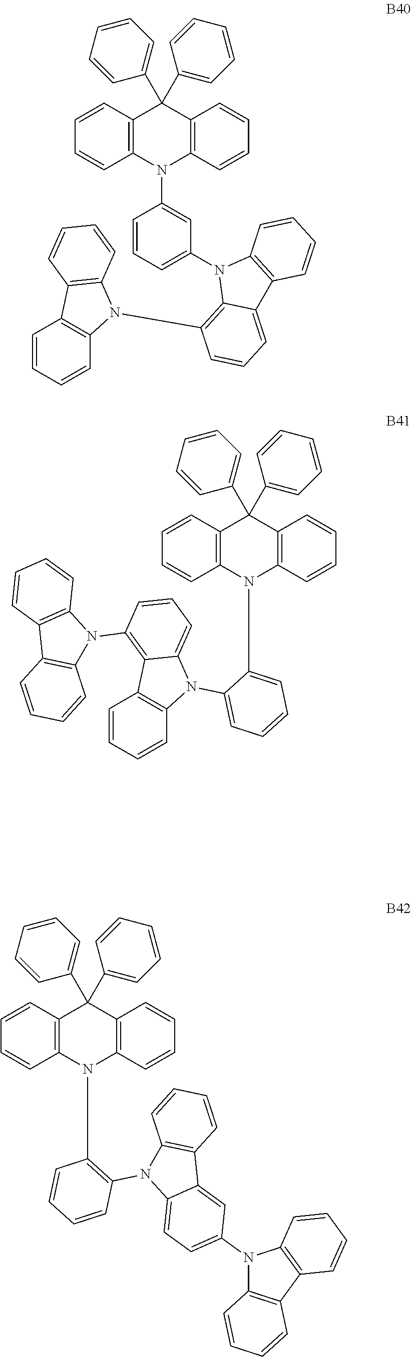

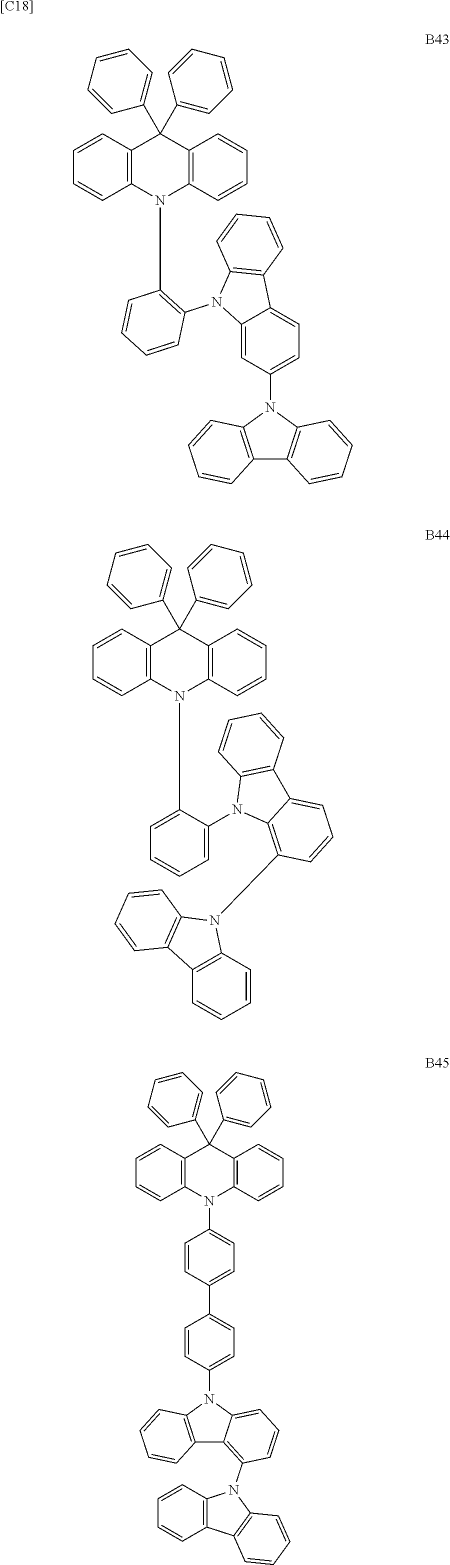

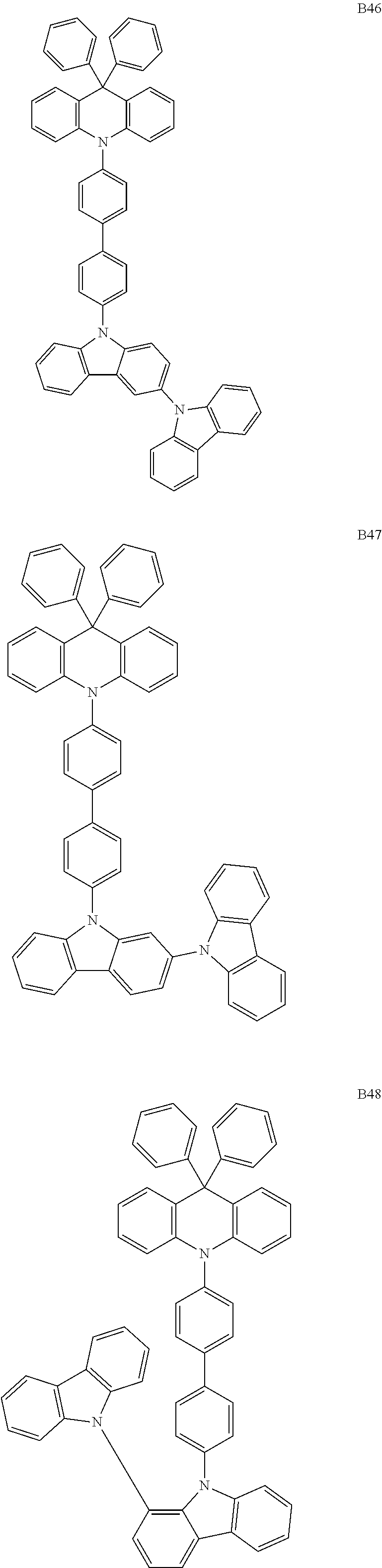

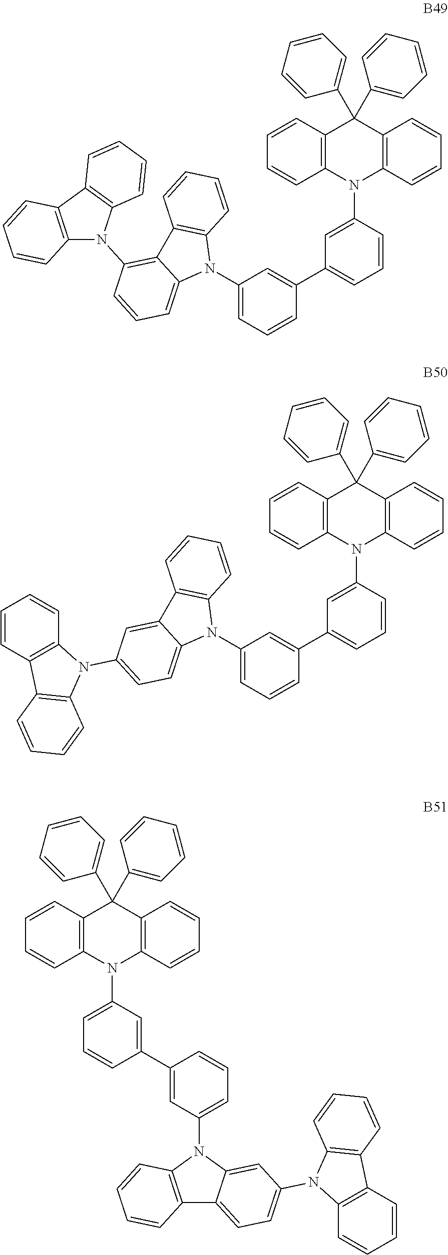

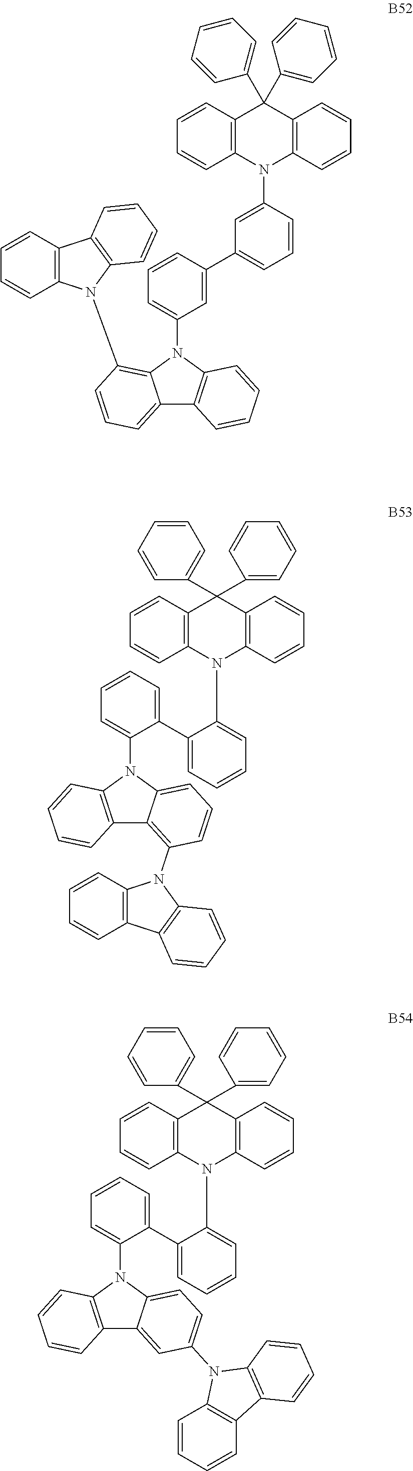

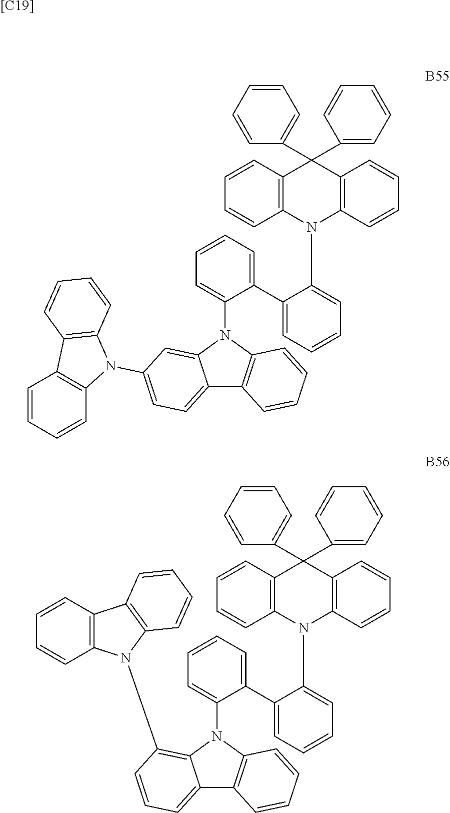

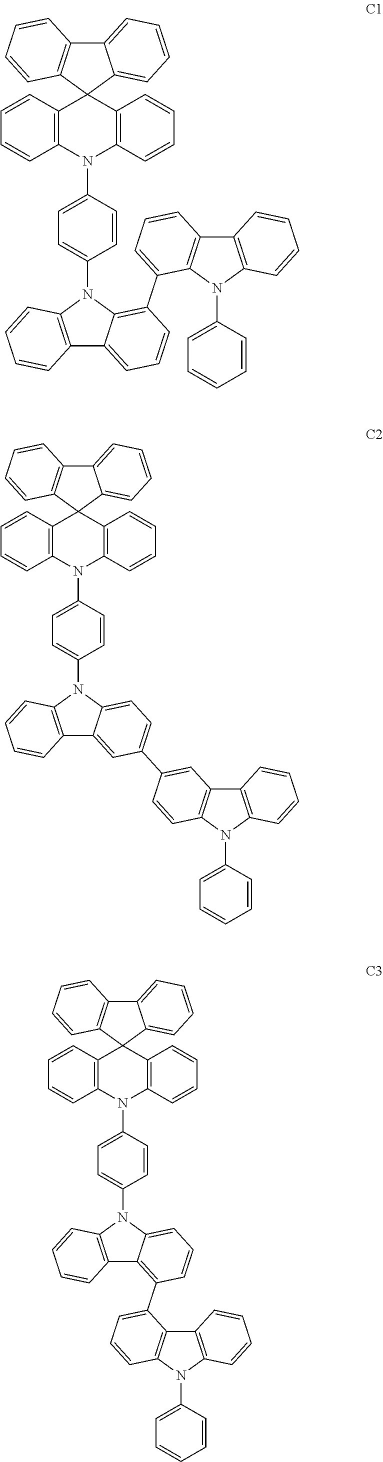

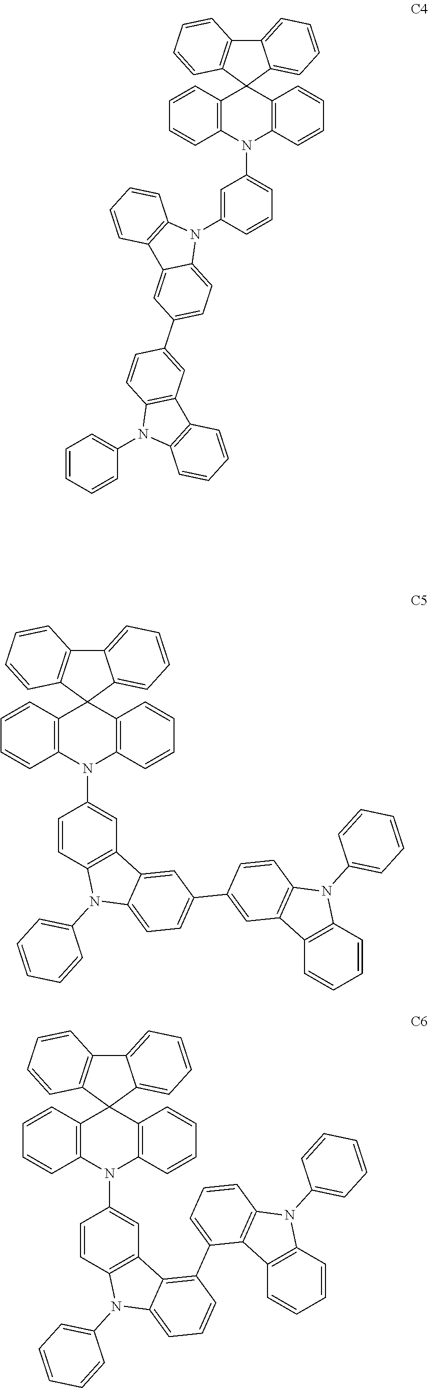

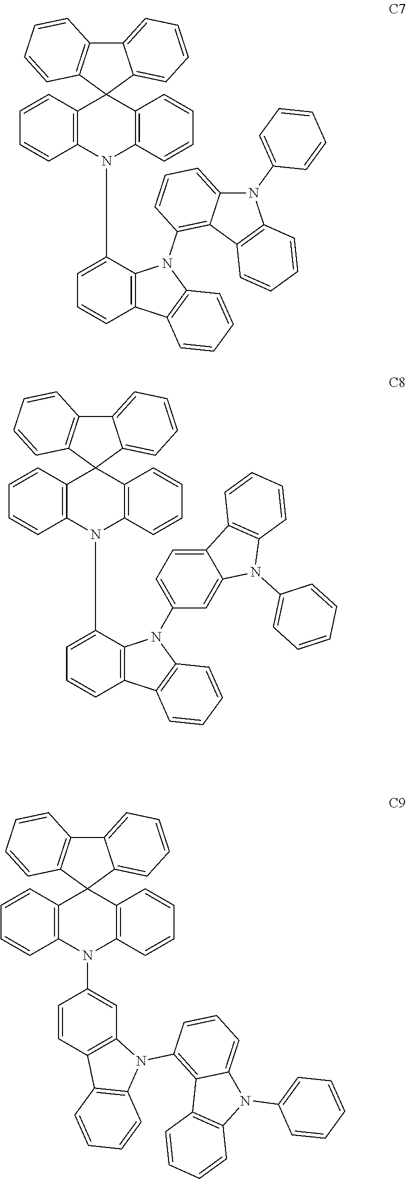

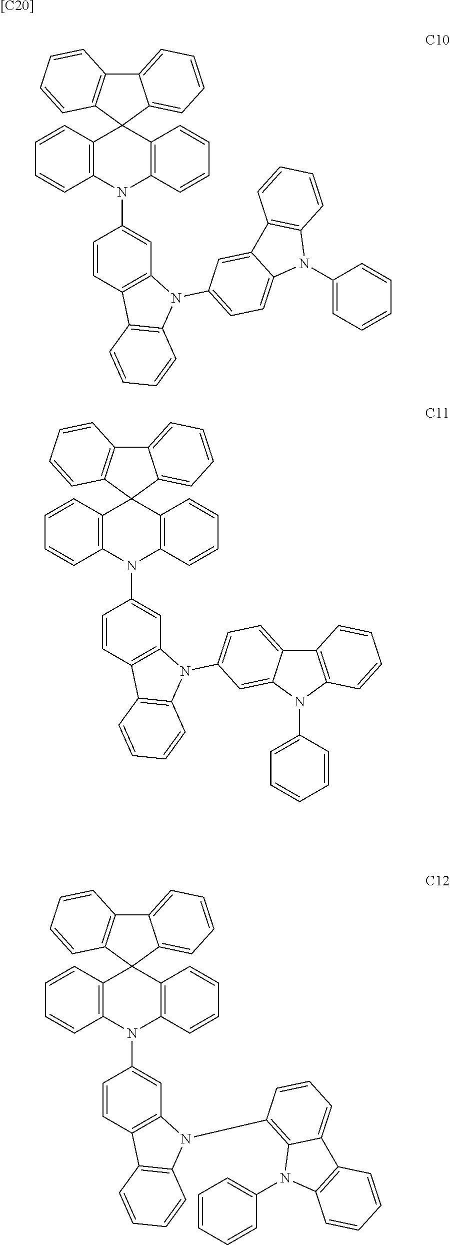

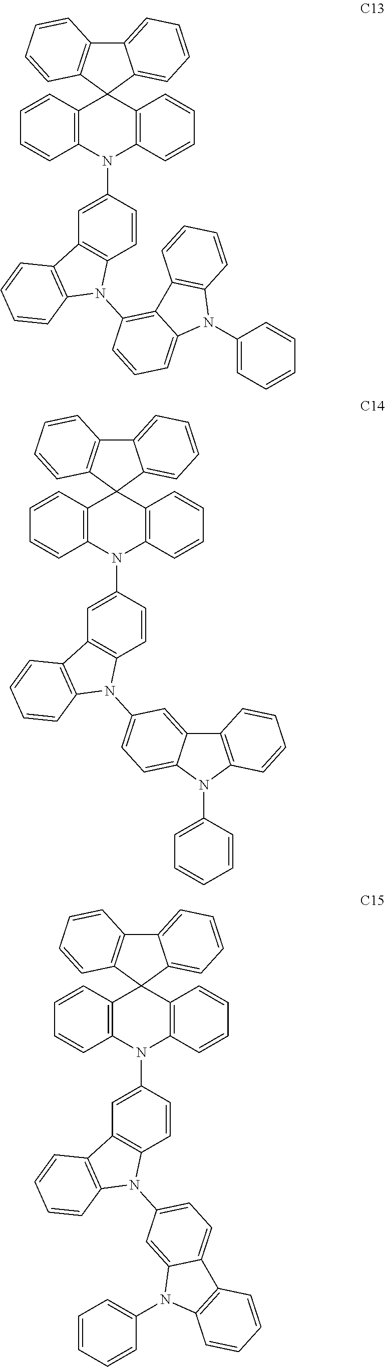

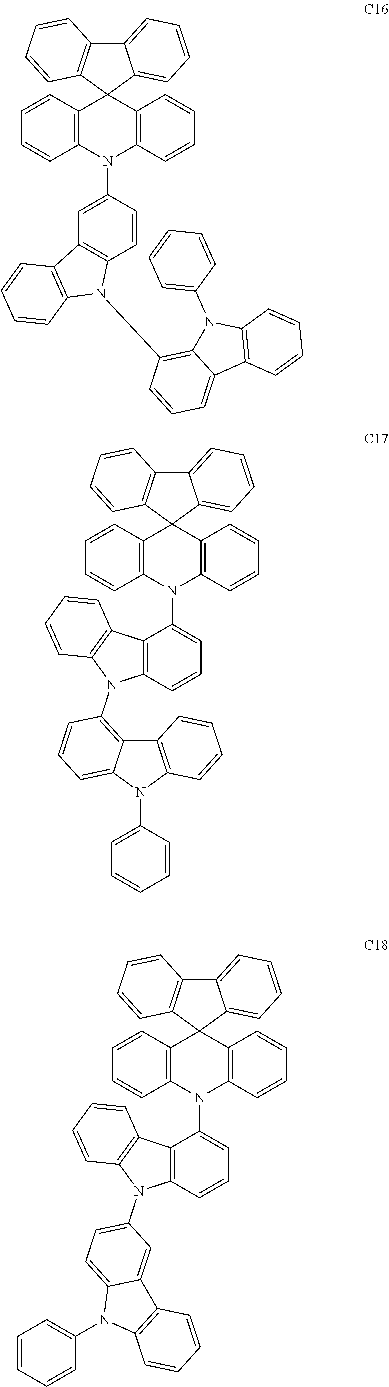

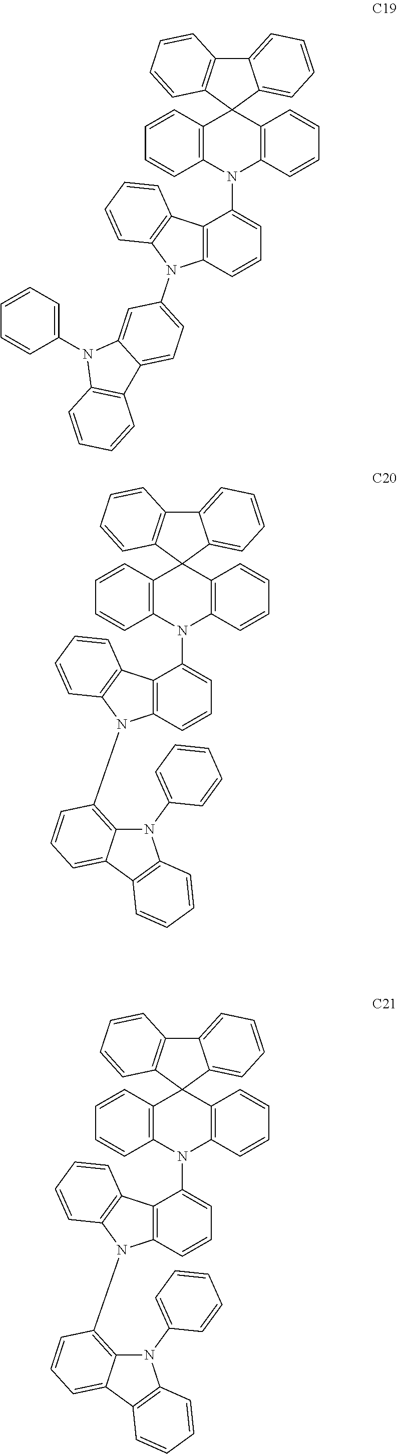

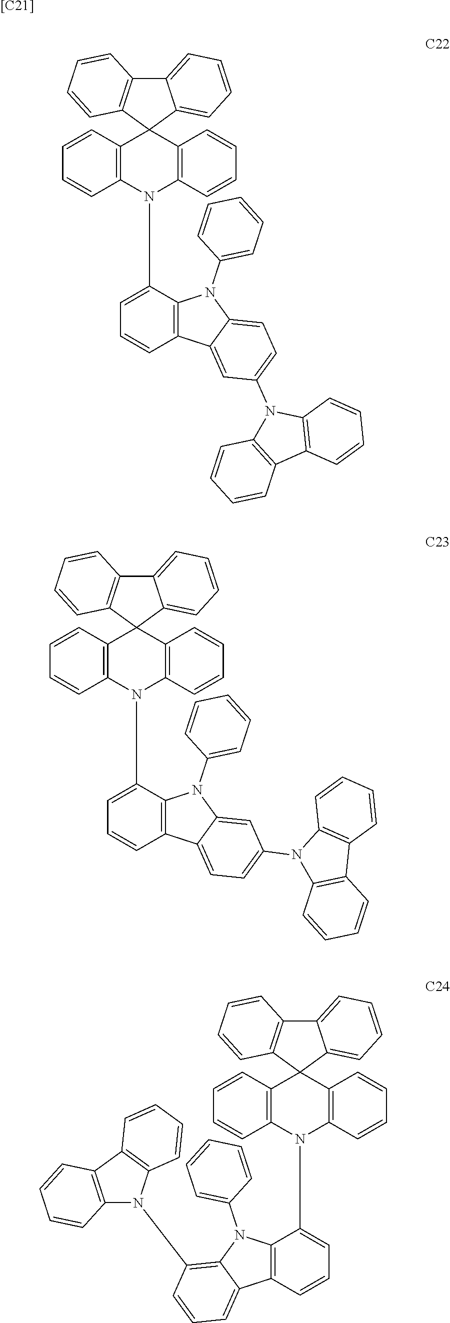

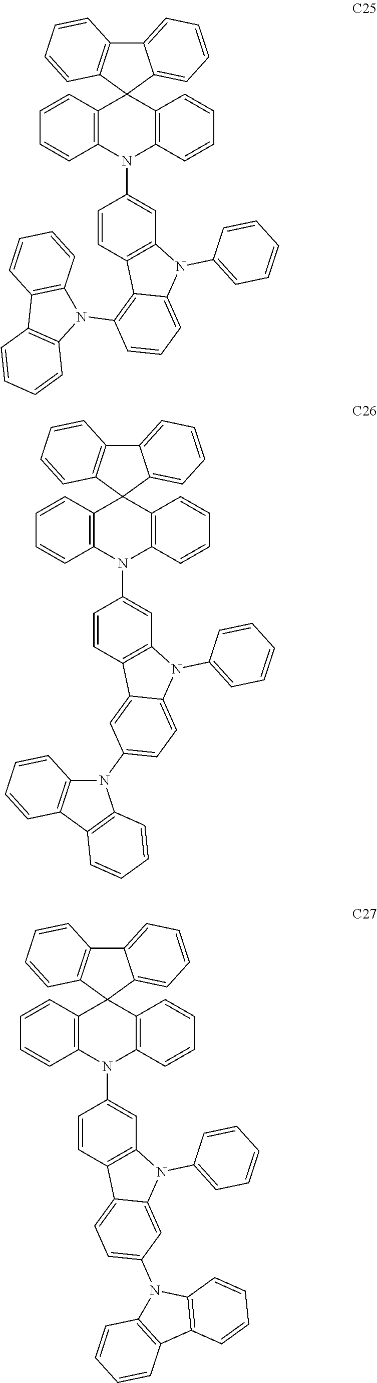

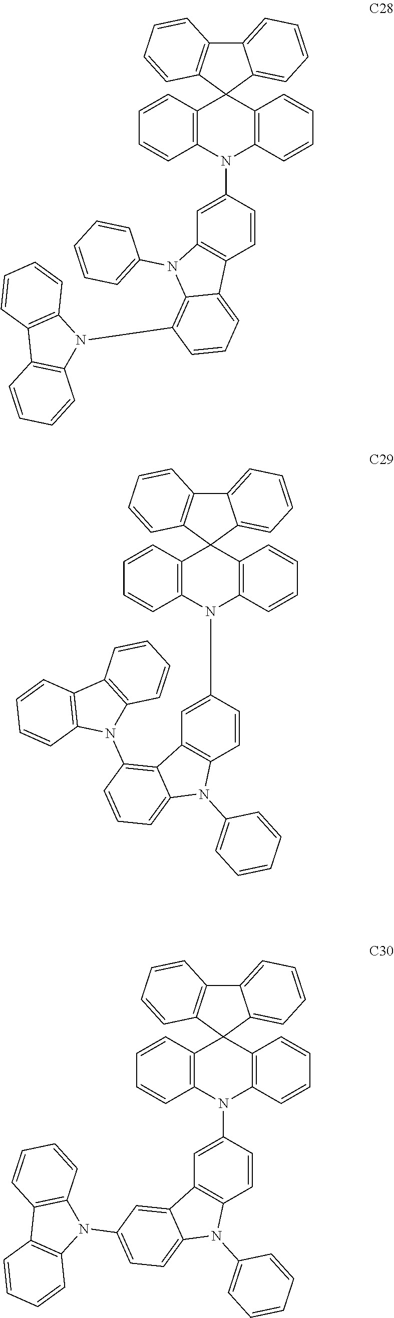

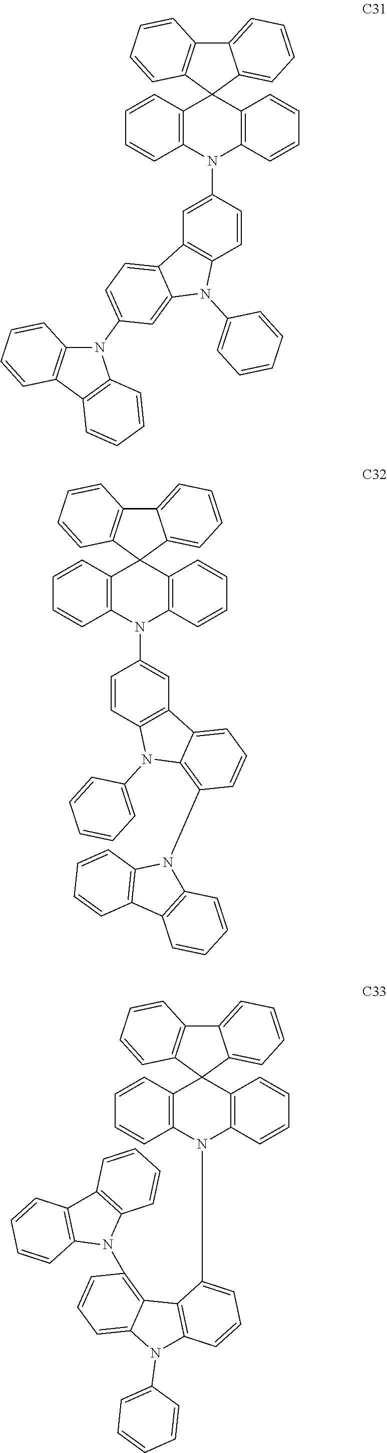

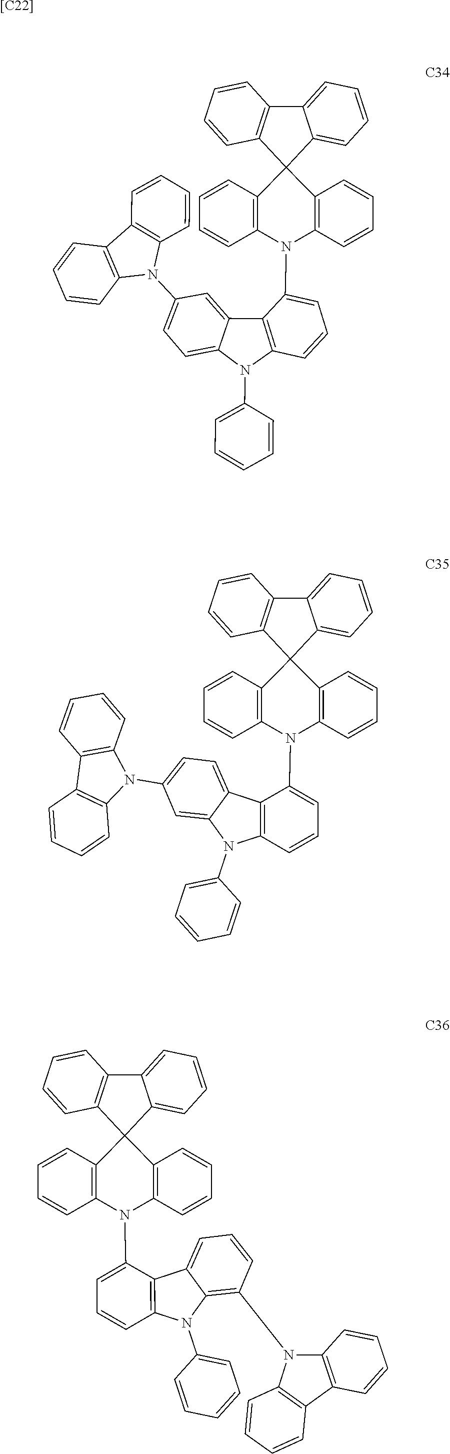

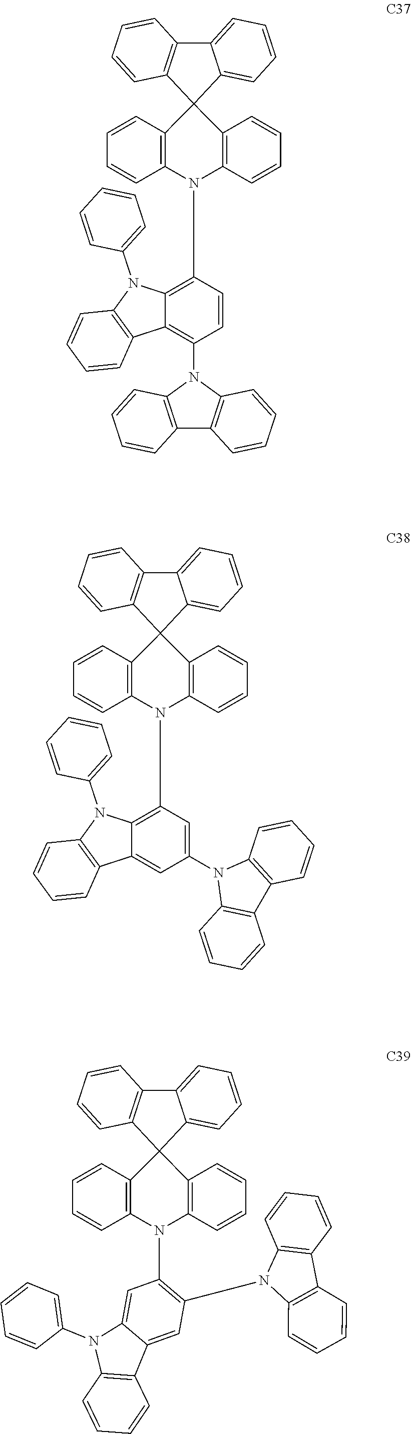

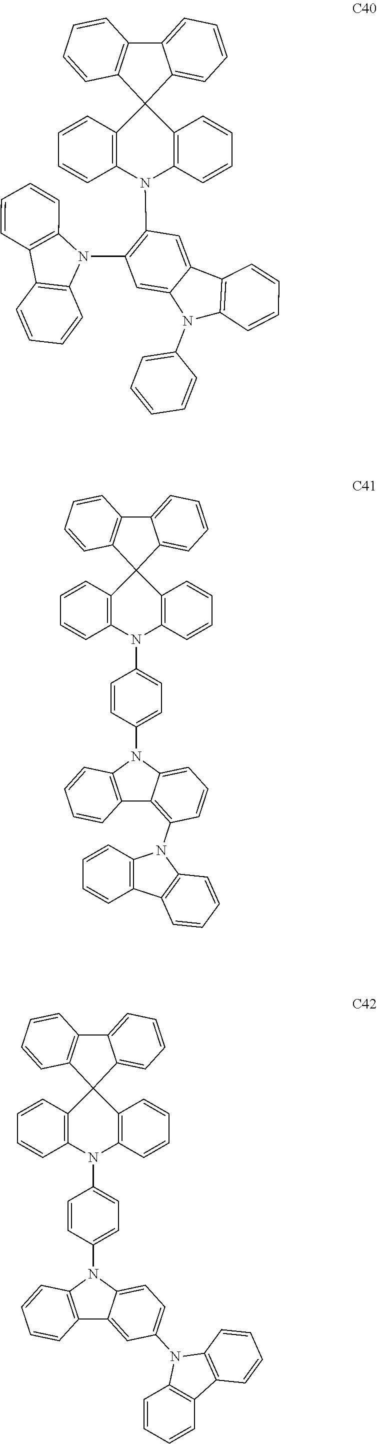

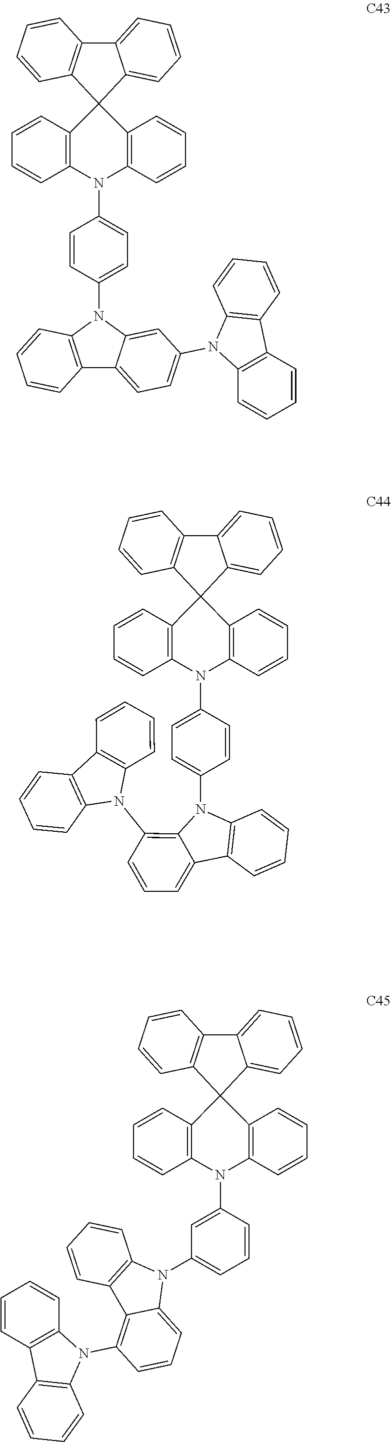

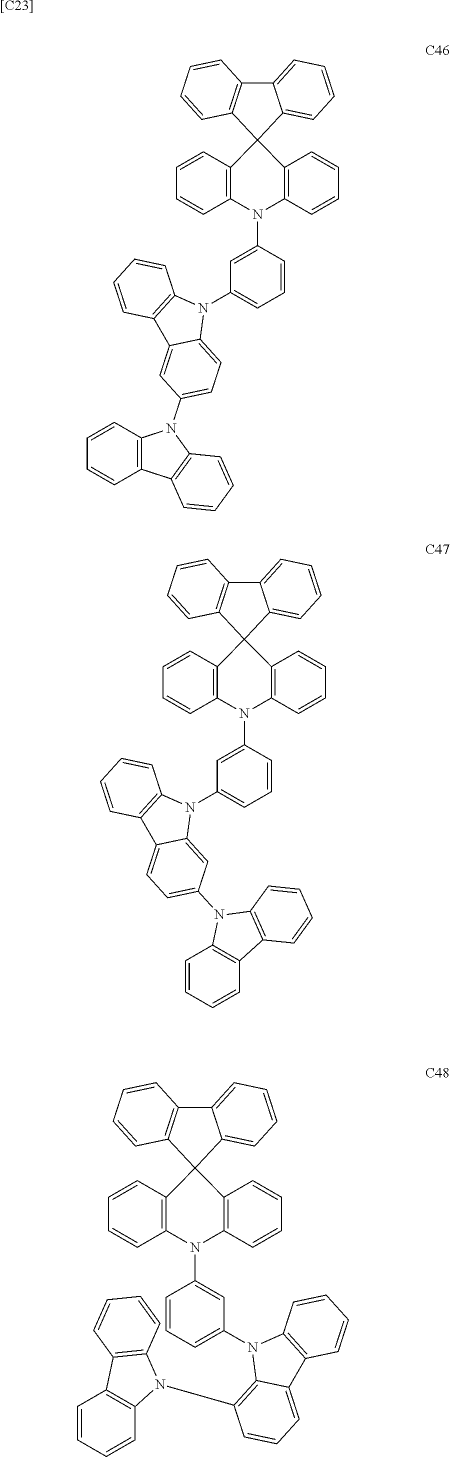

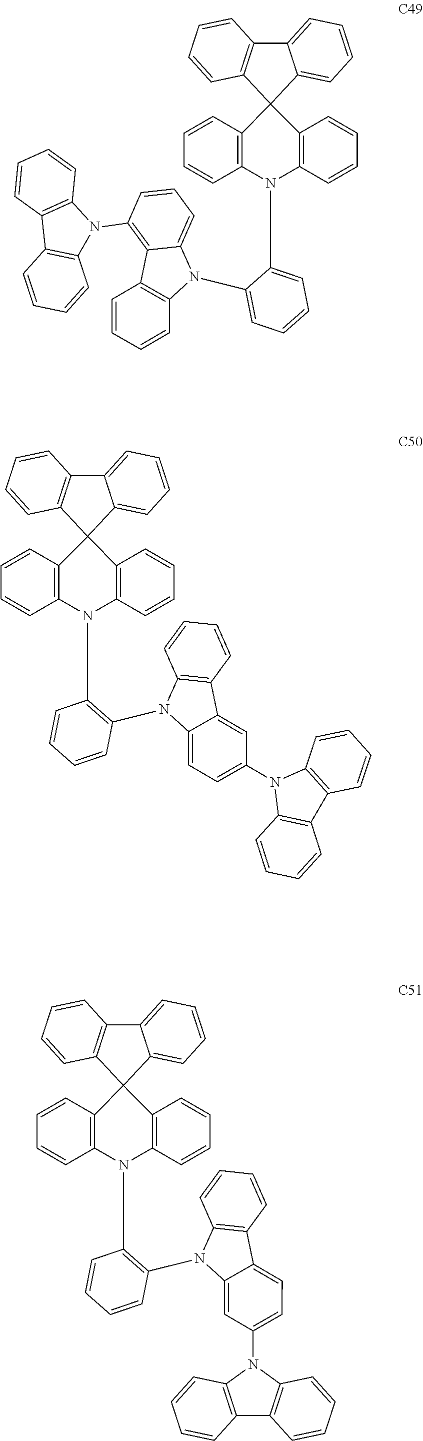

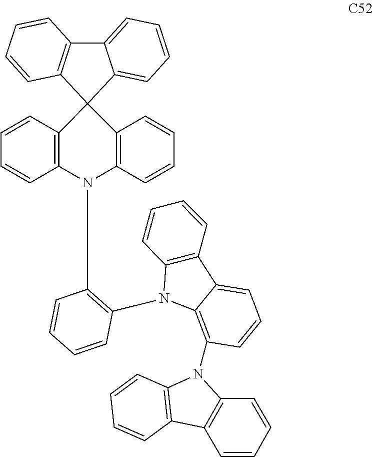

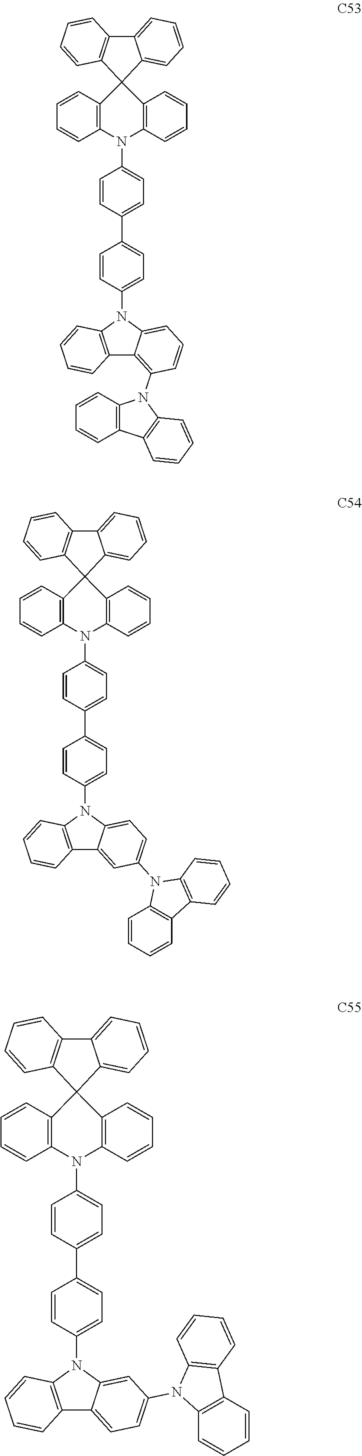

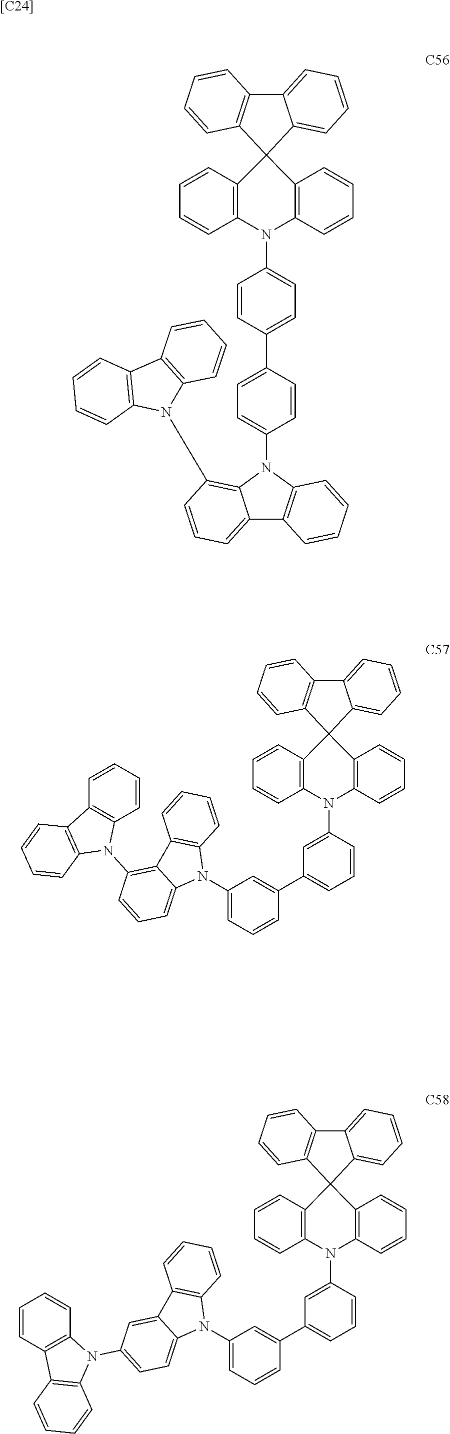

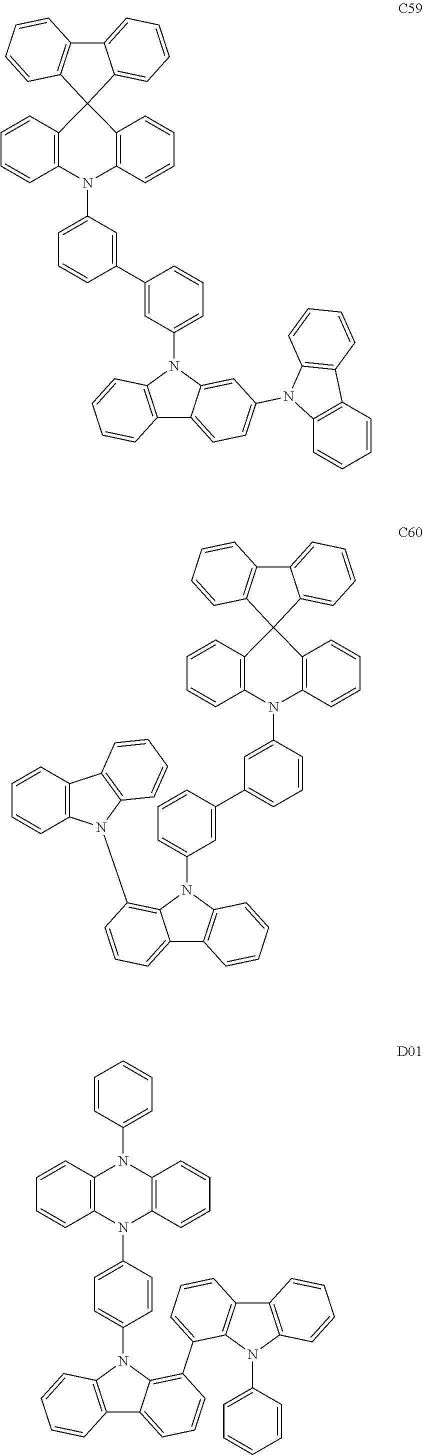

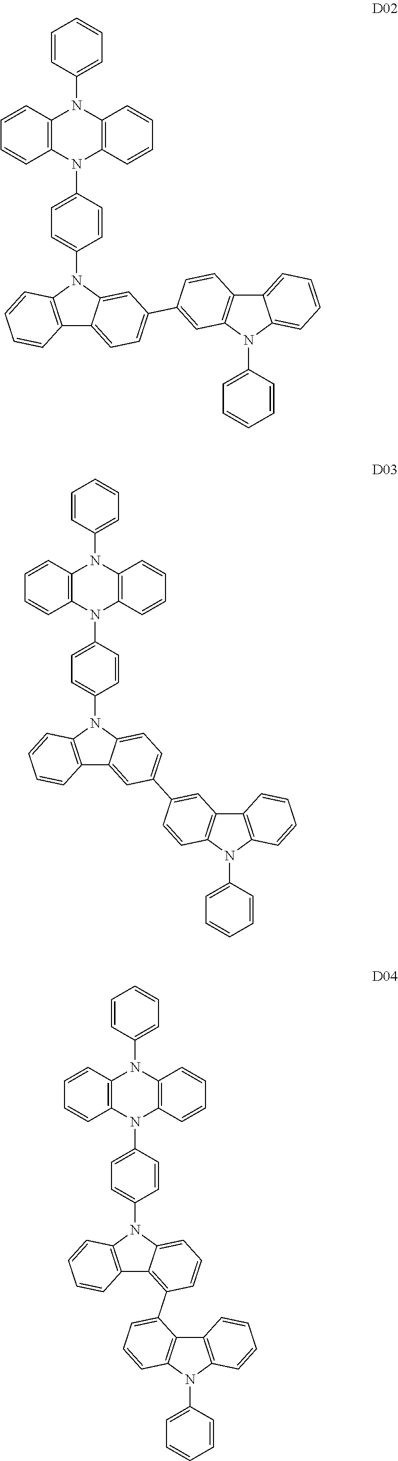

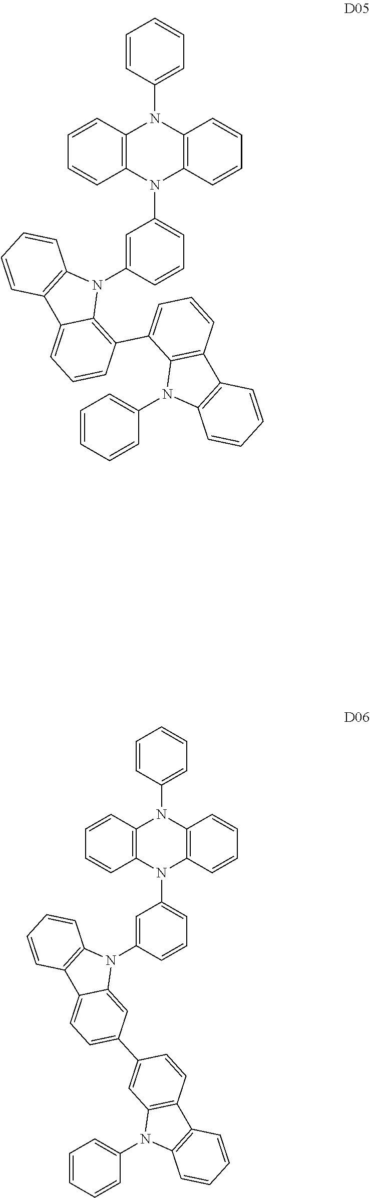

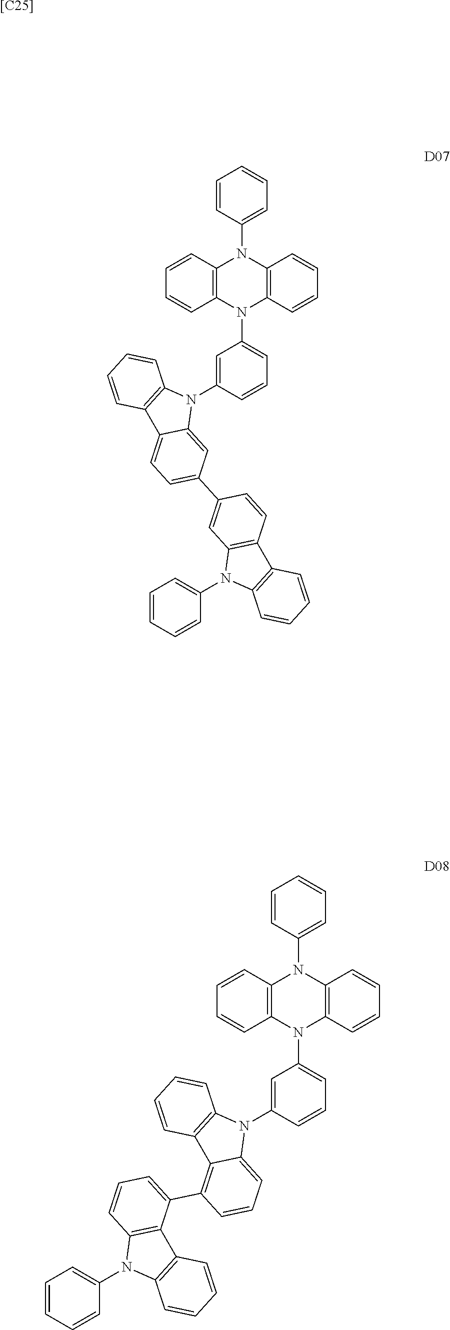

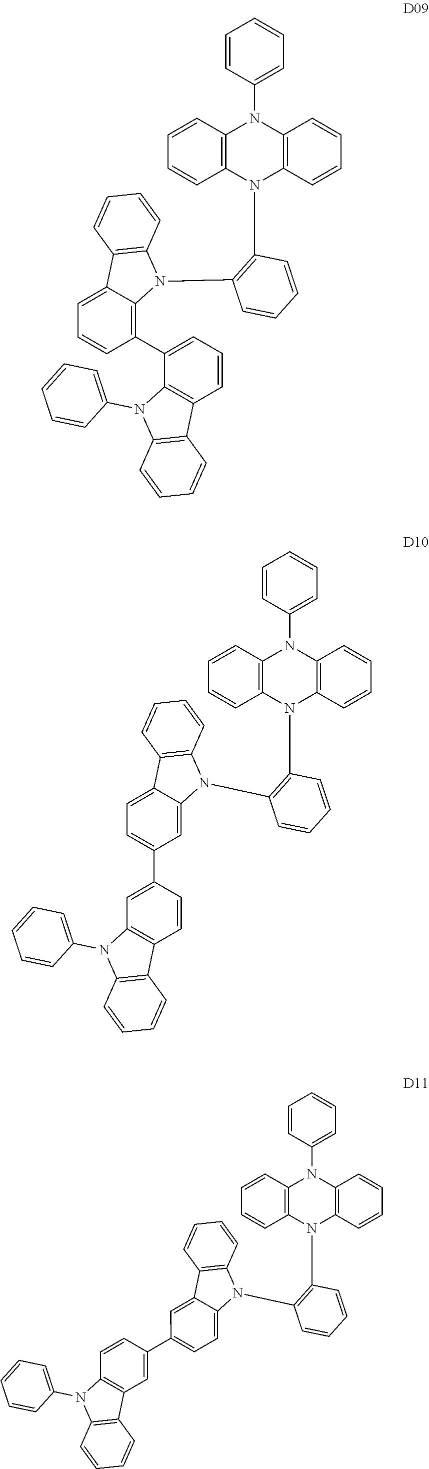

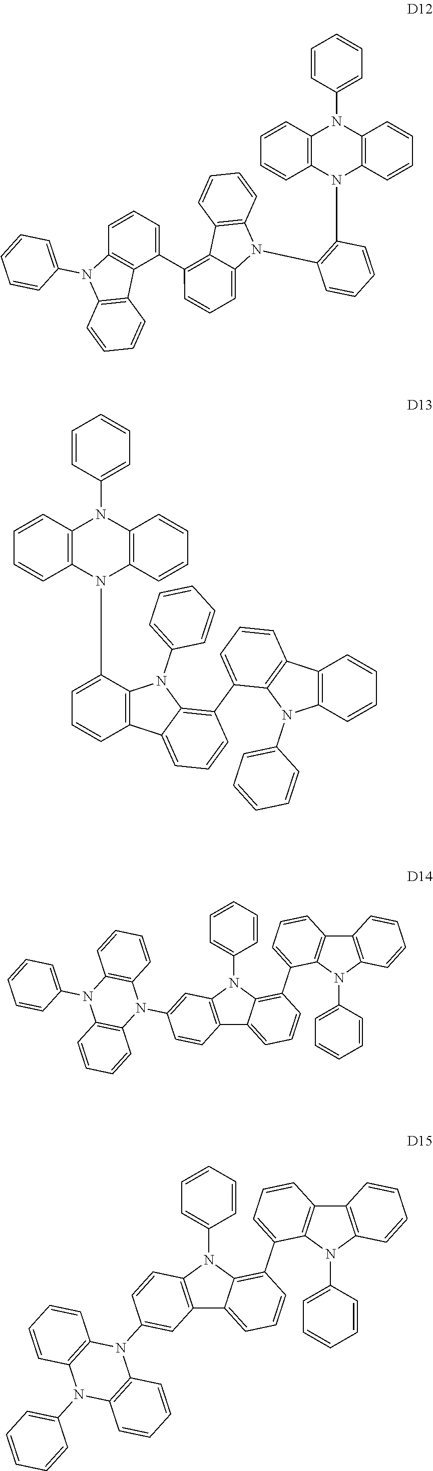

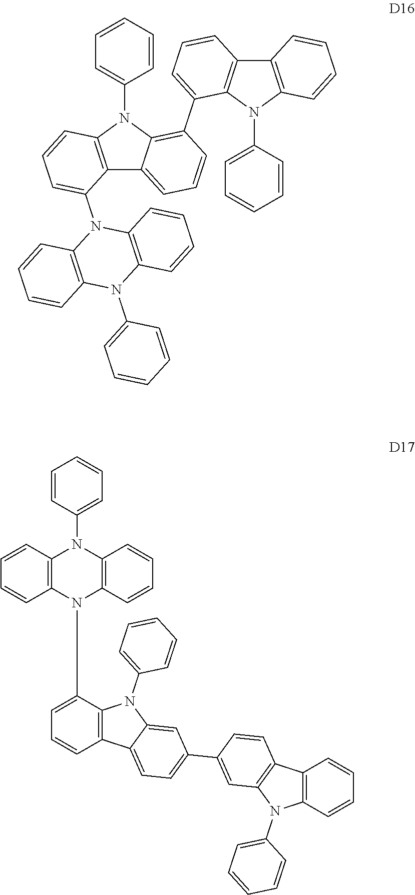

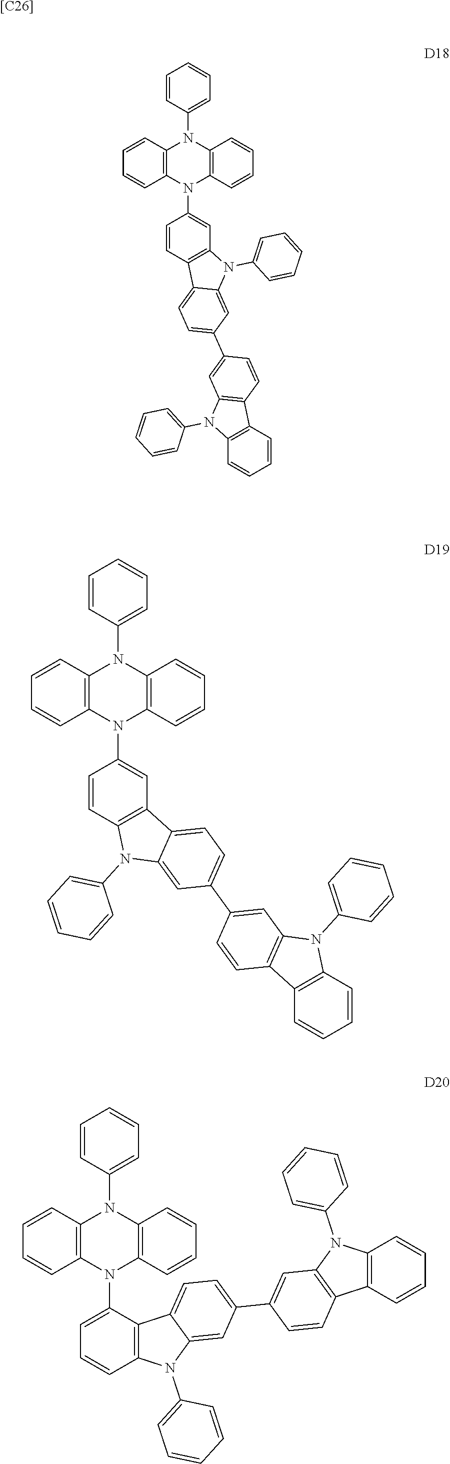

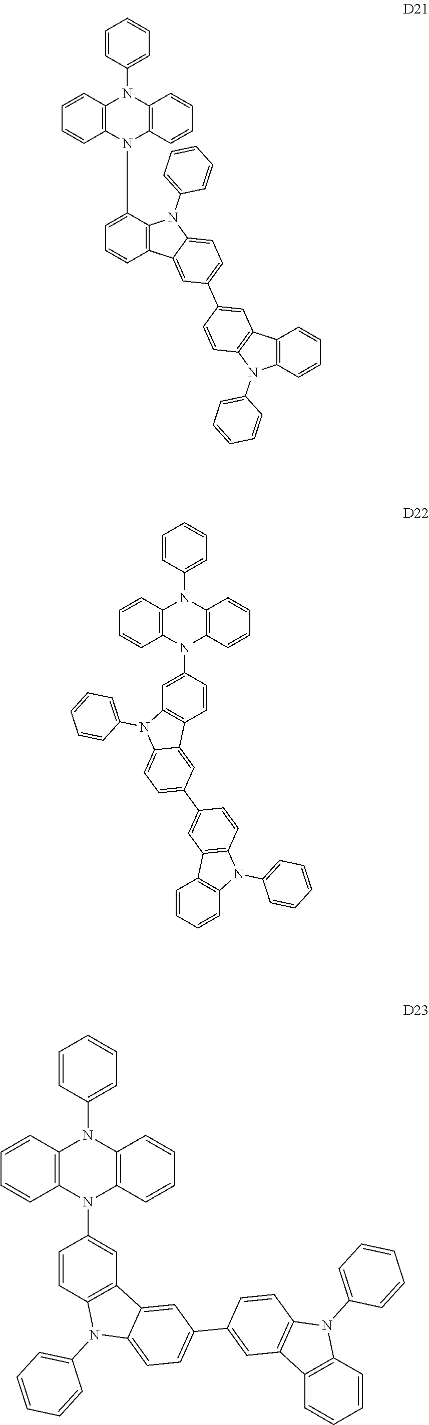

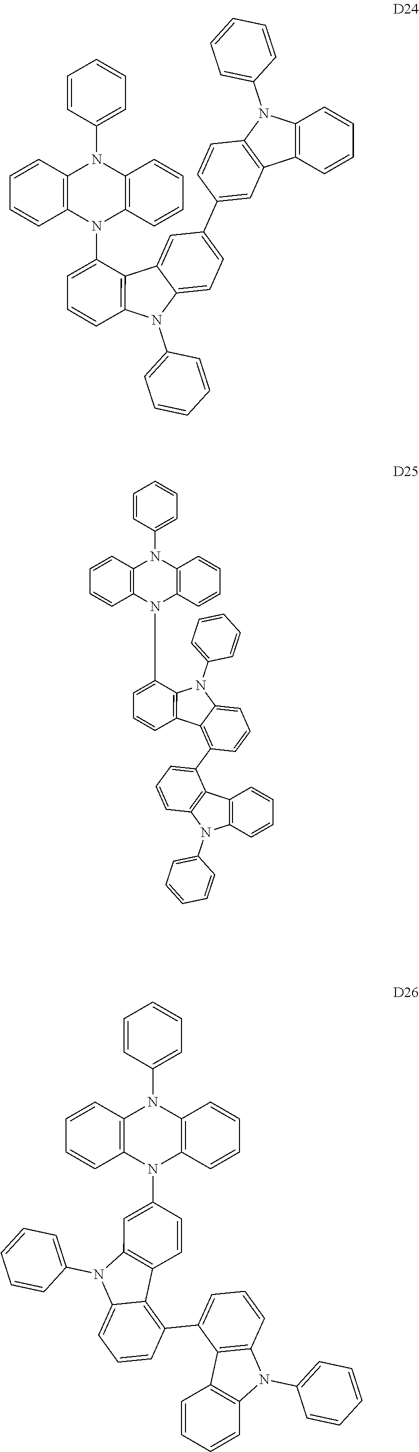

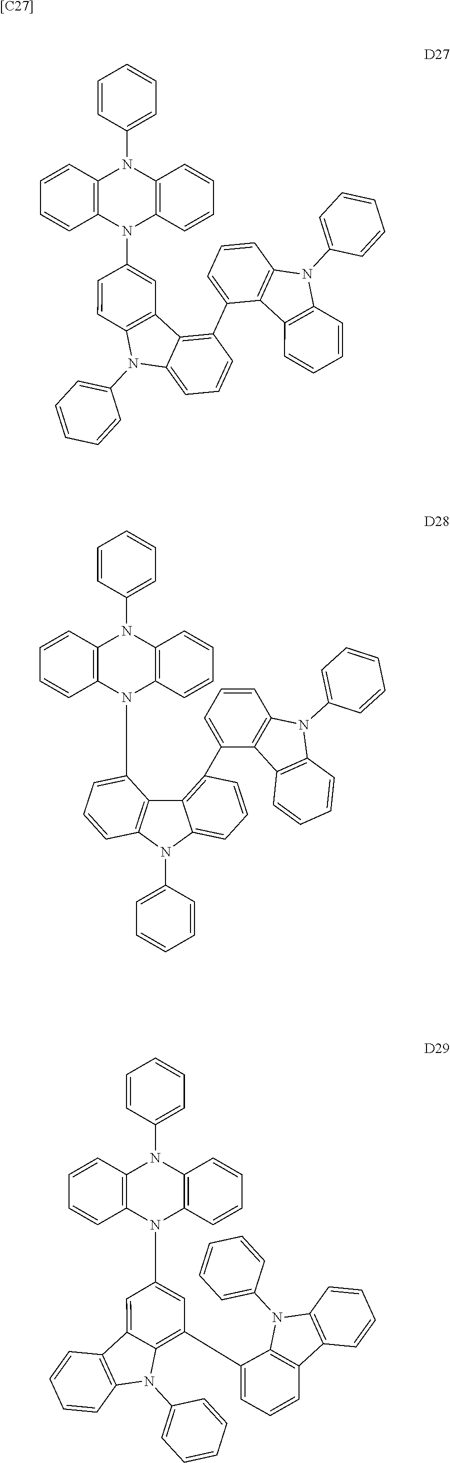

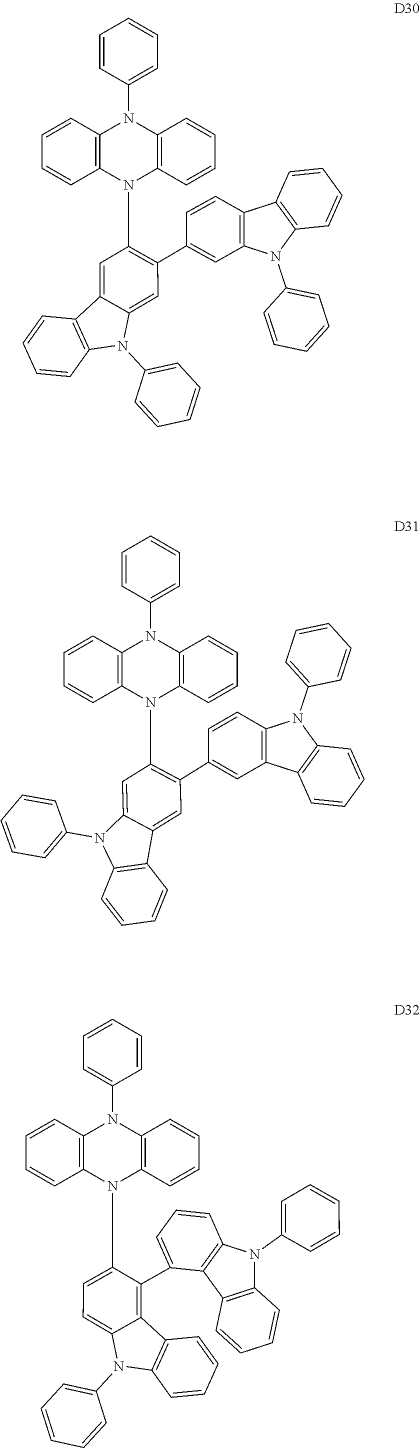

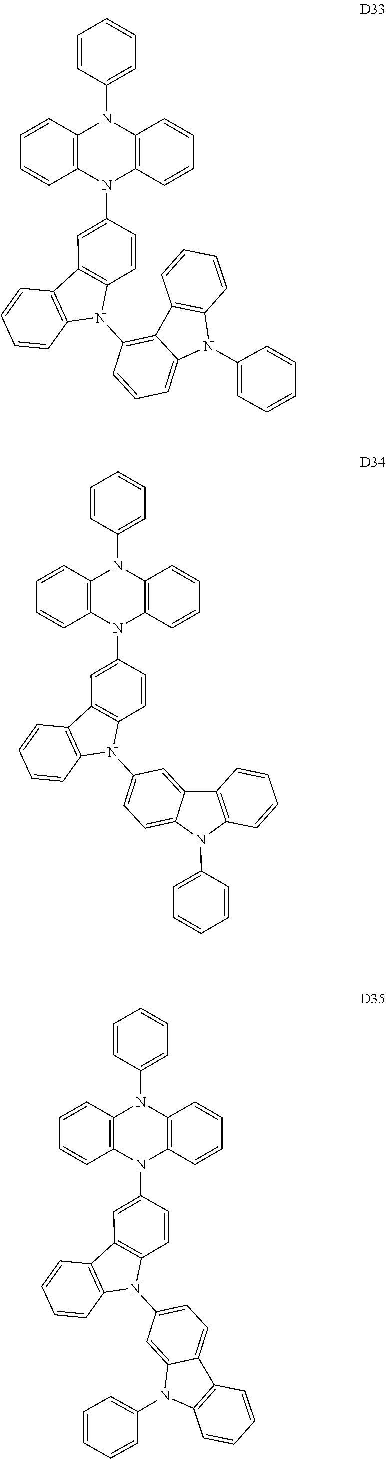

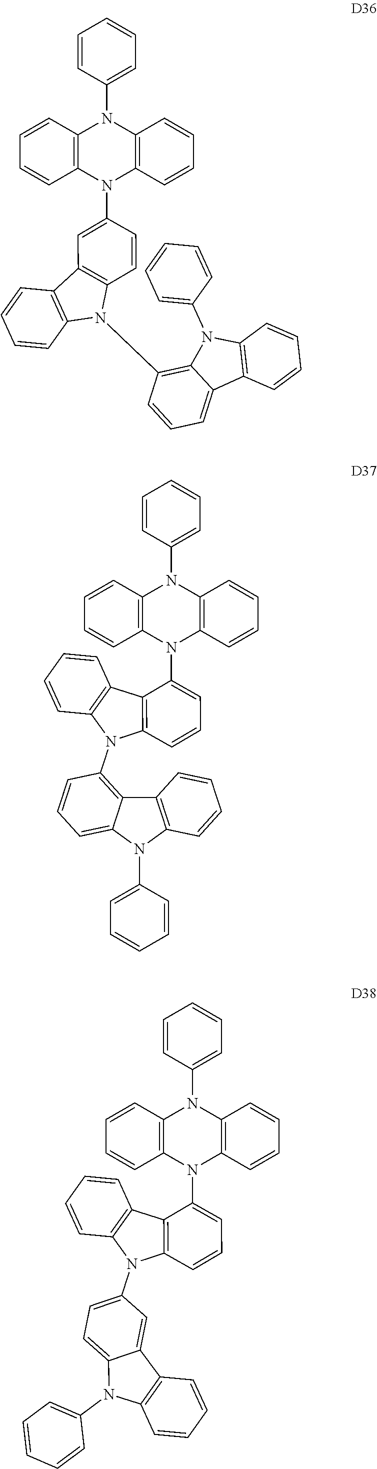

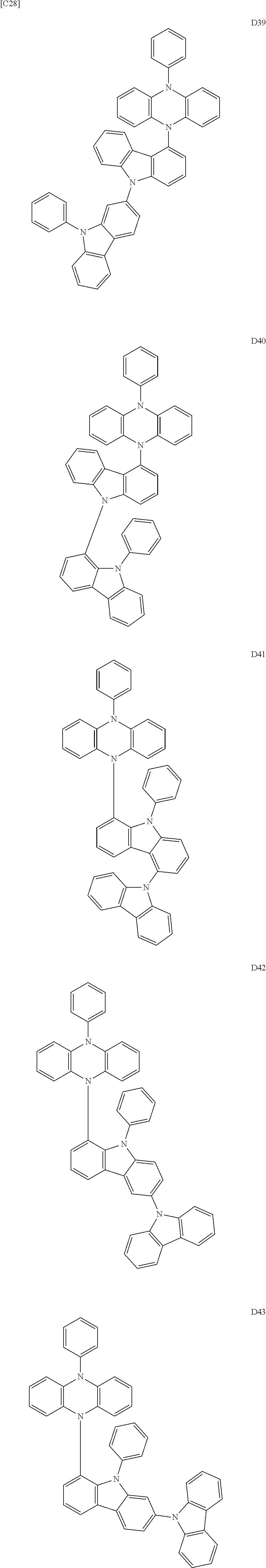

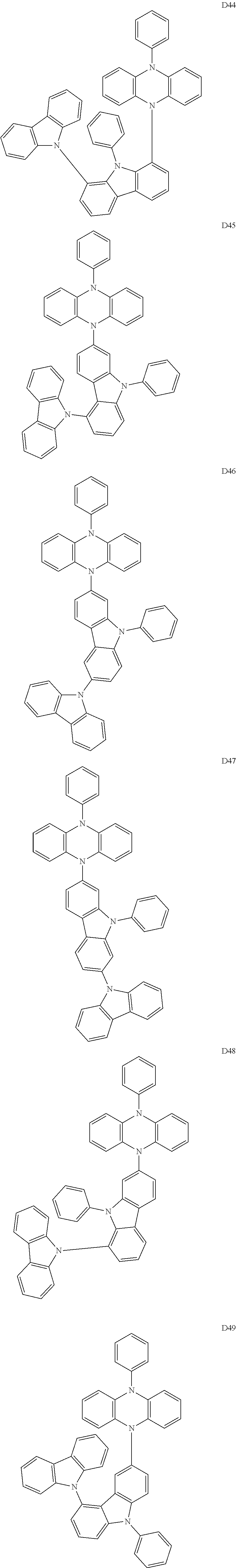

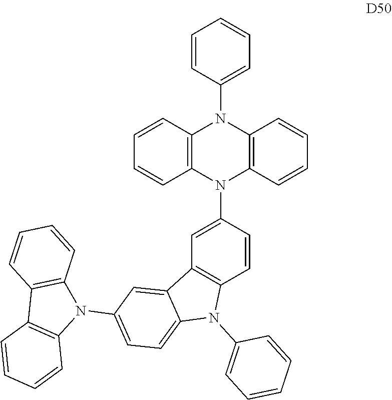

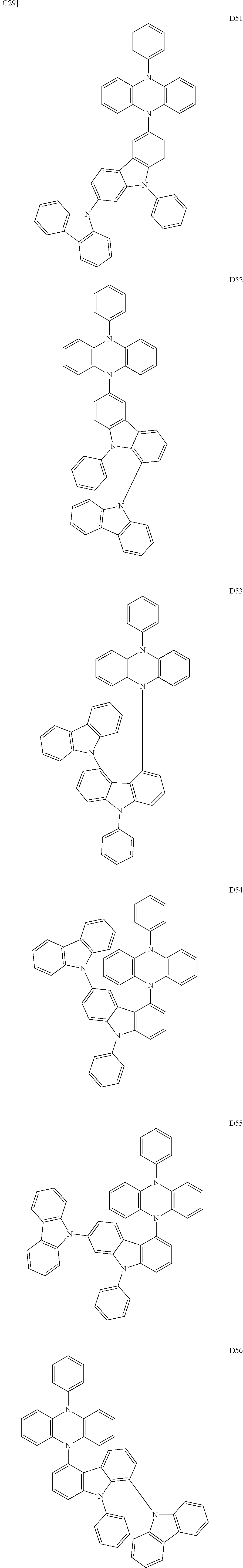

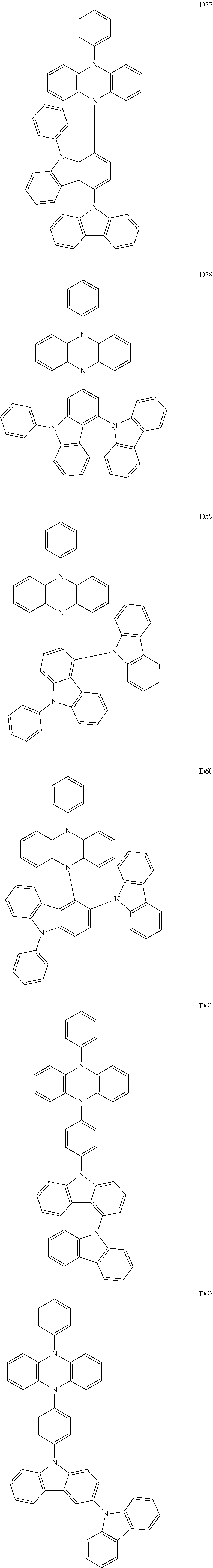

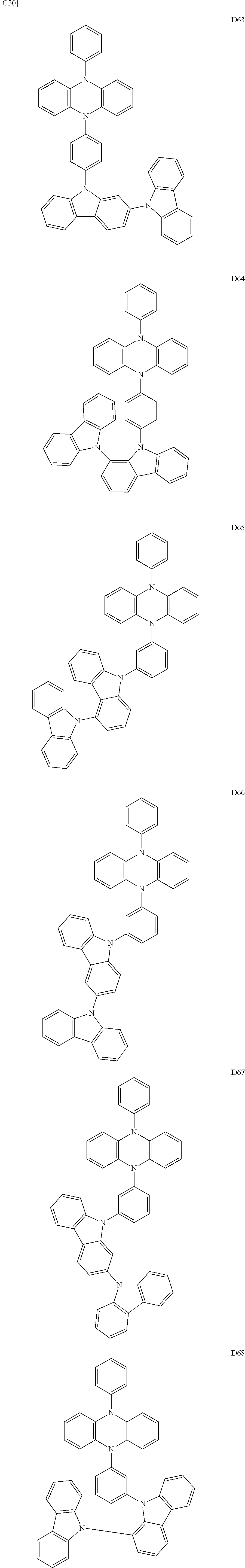

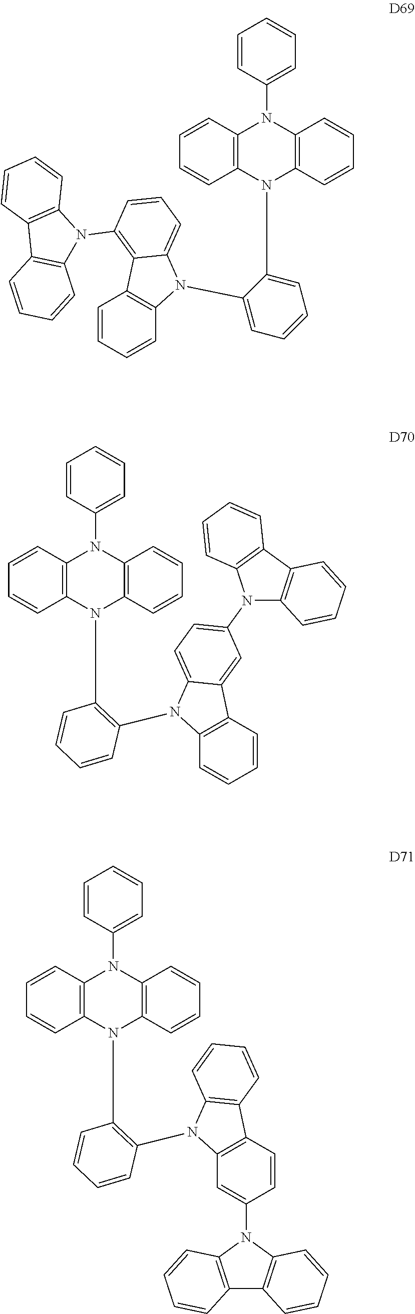

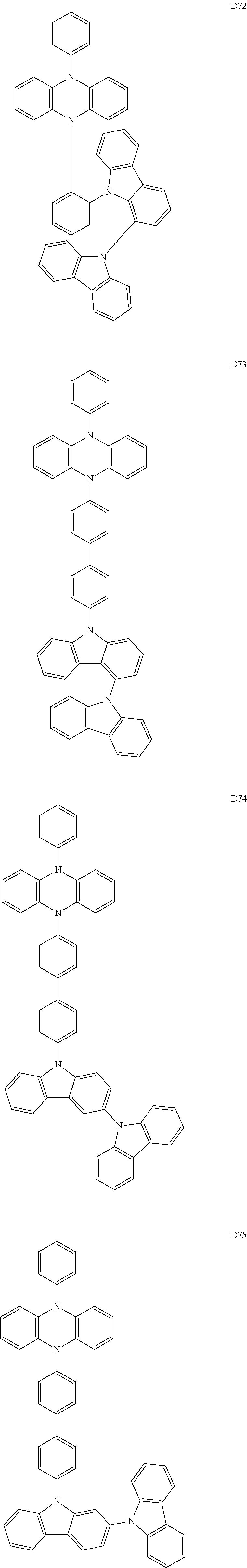

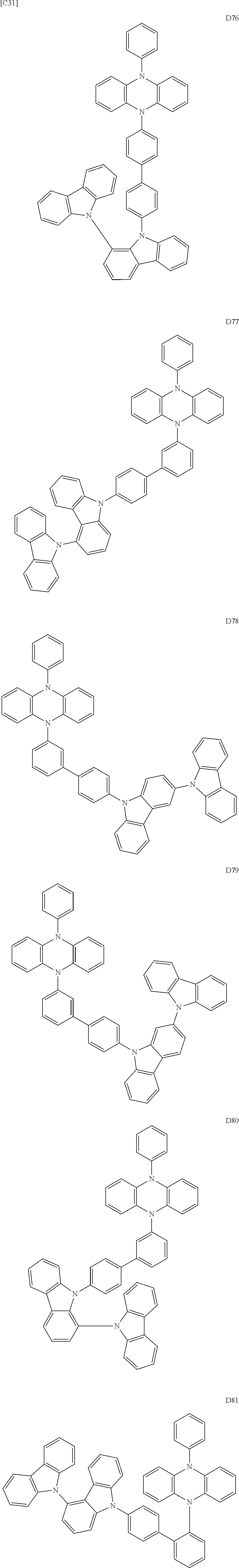

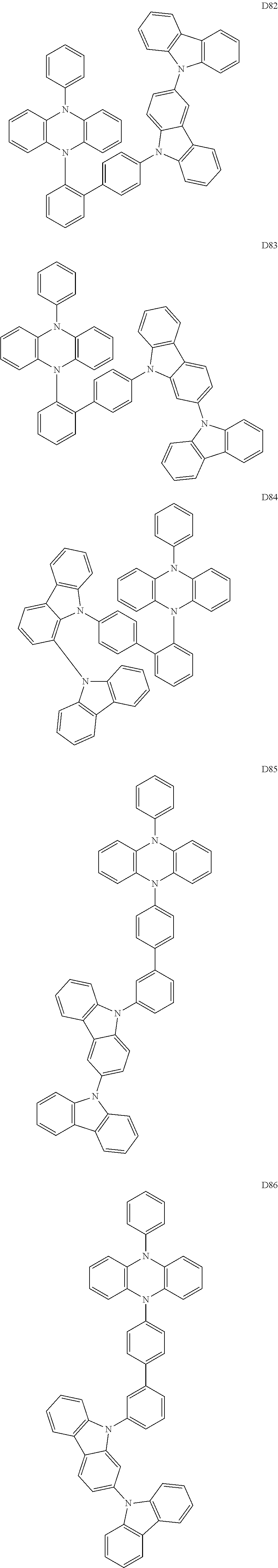

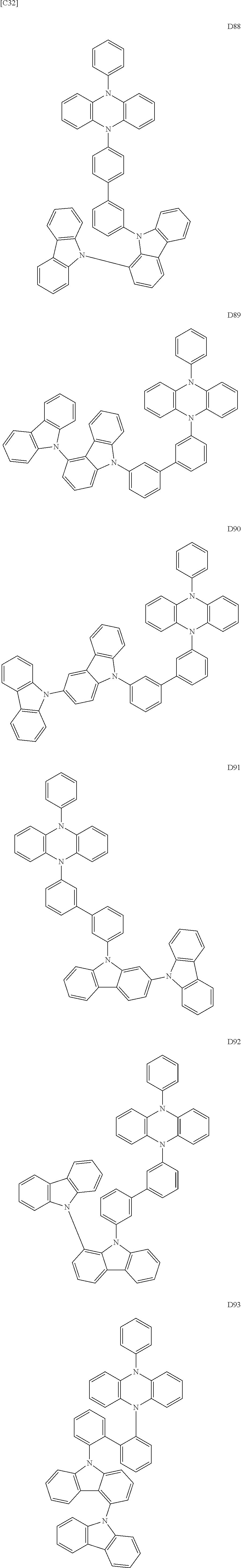

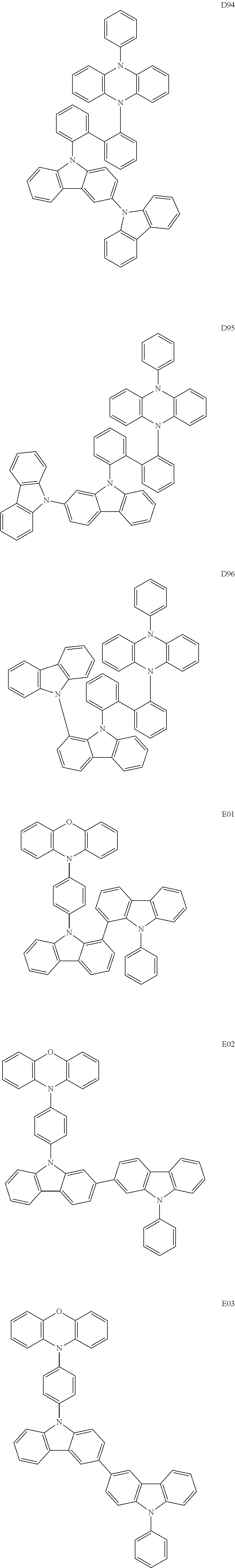

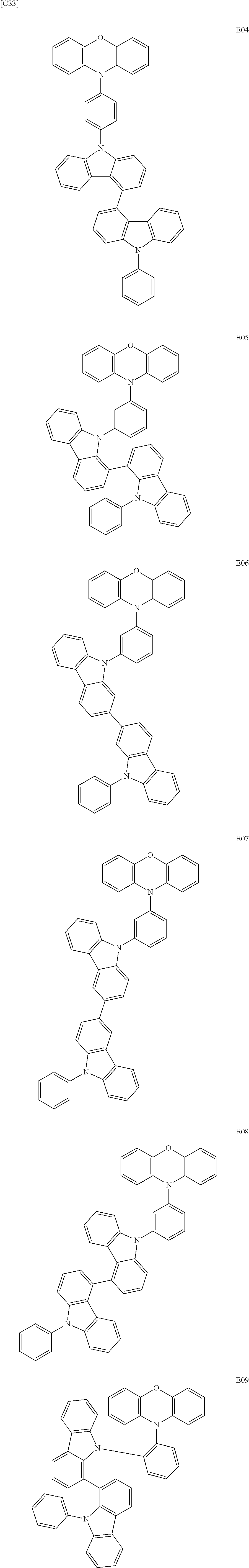

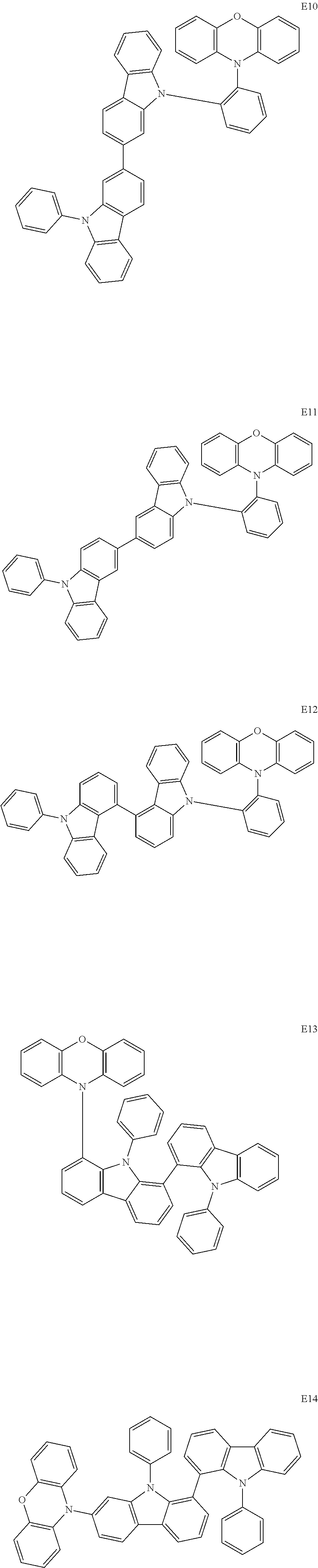

[chemical structure]

[chemical structure]

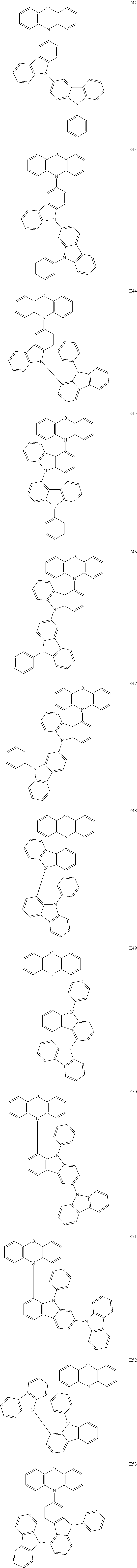

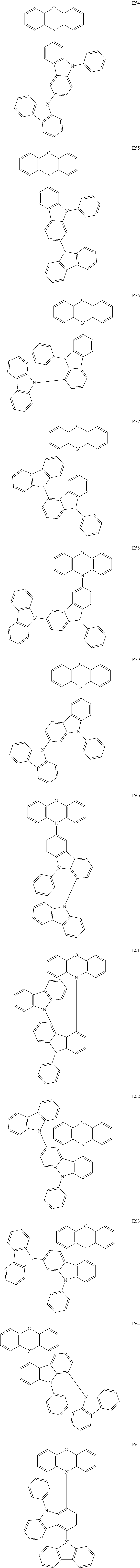

[chemical structure]

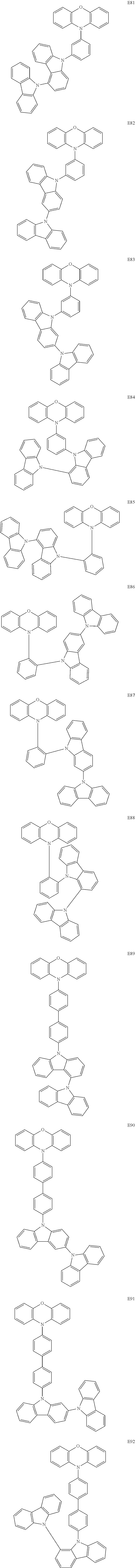

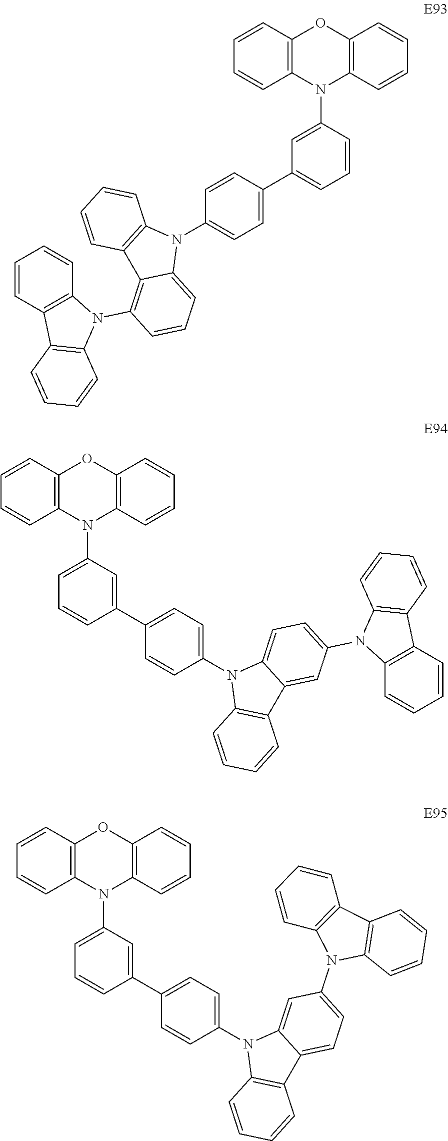

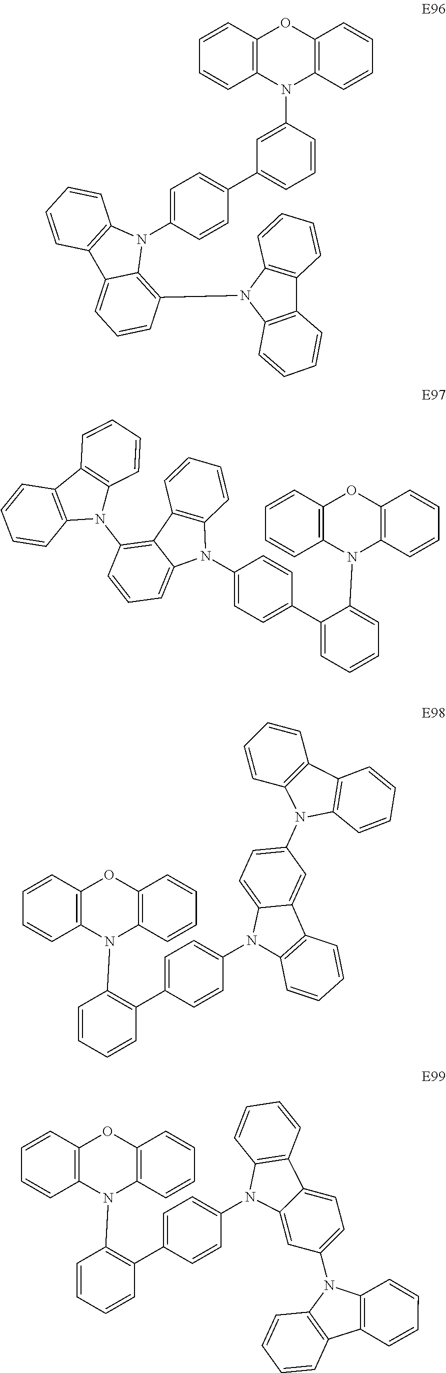

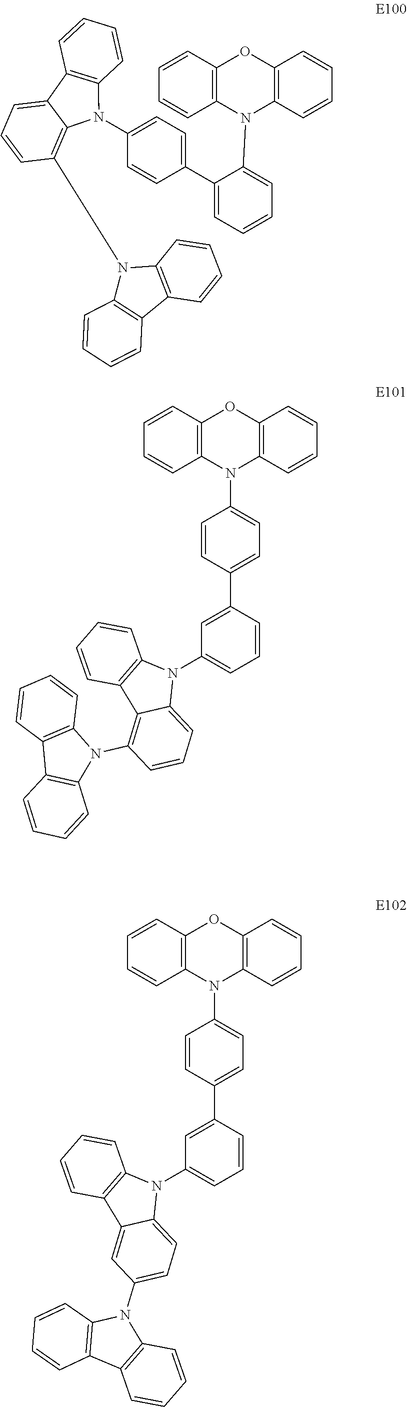

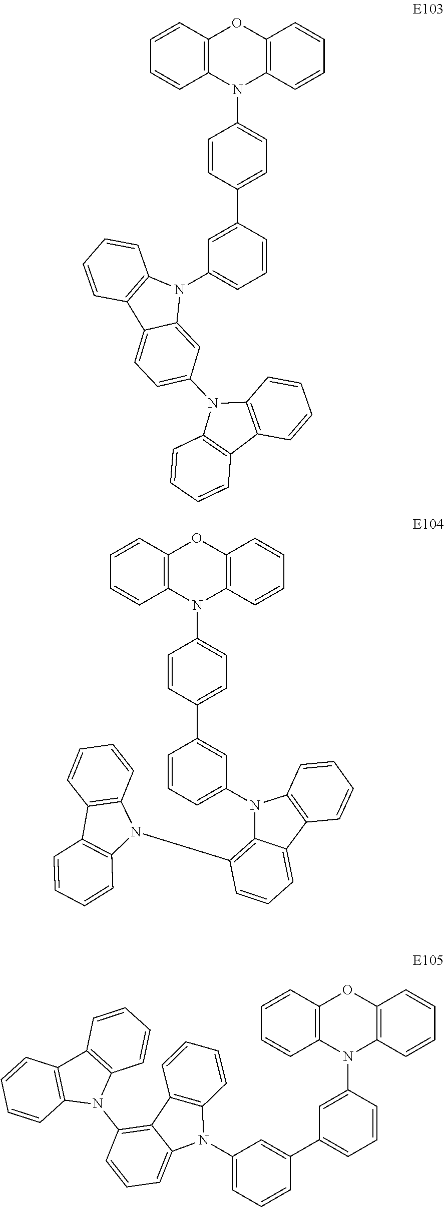

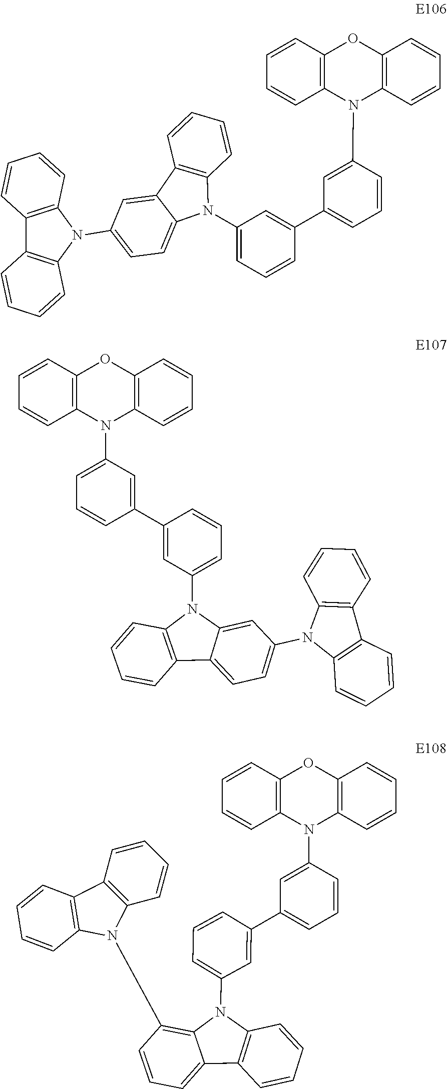

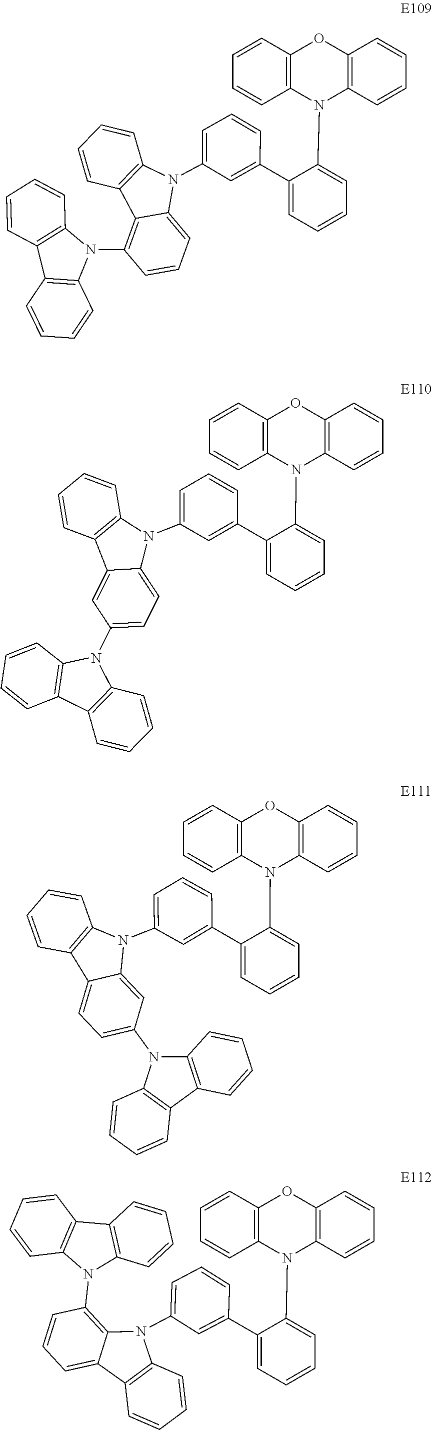

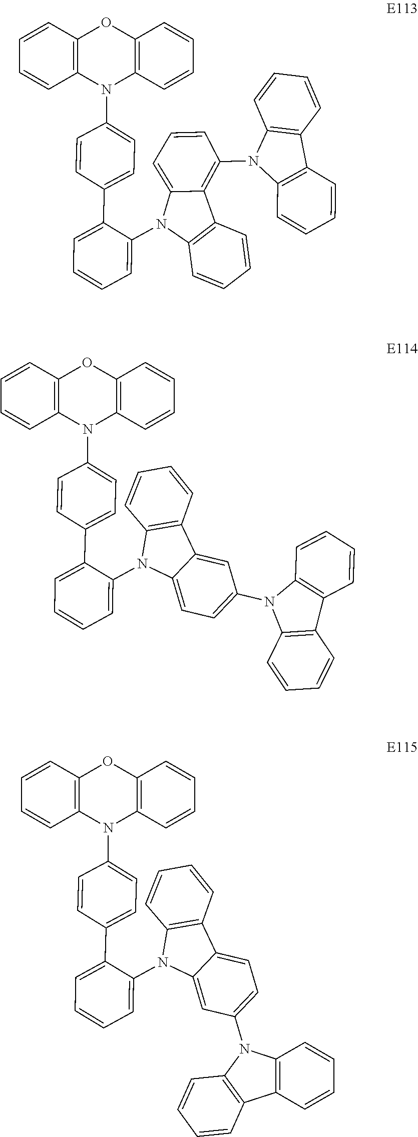

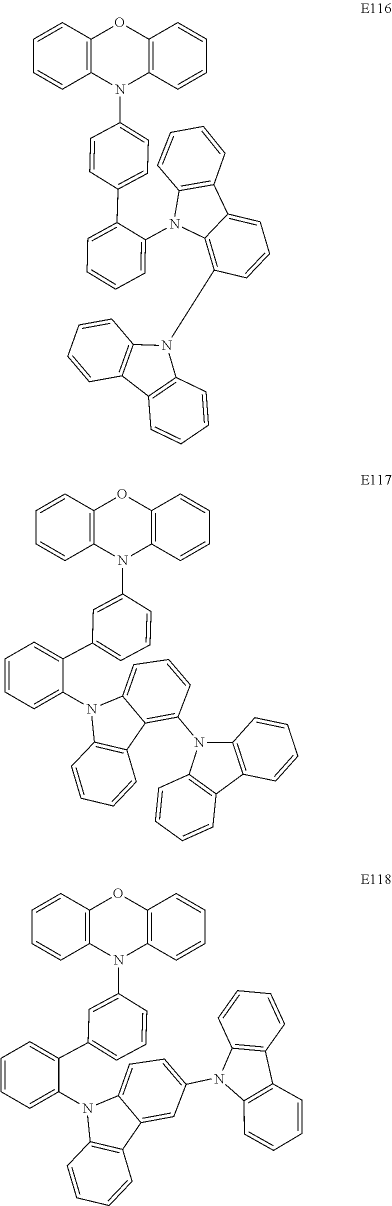

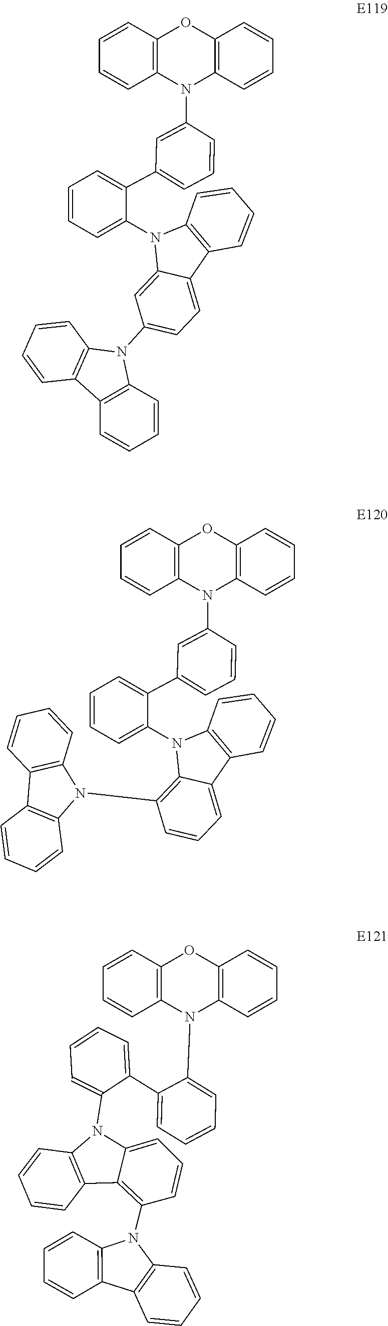

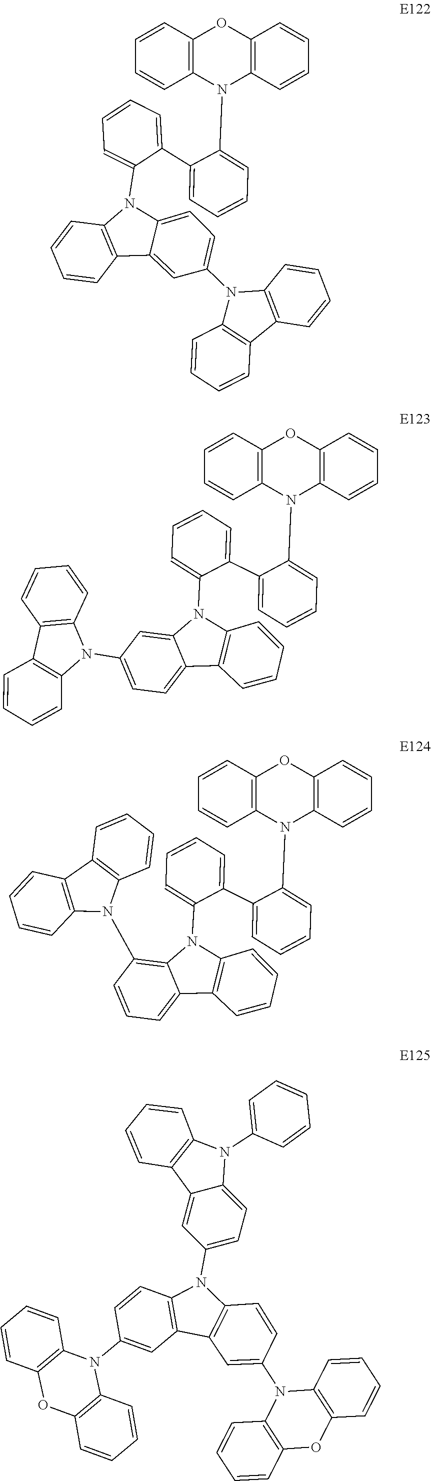

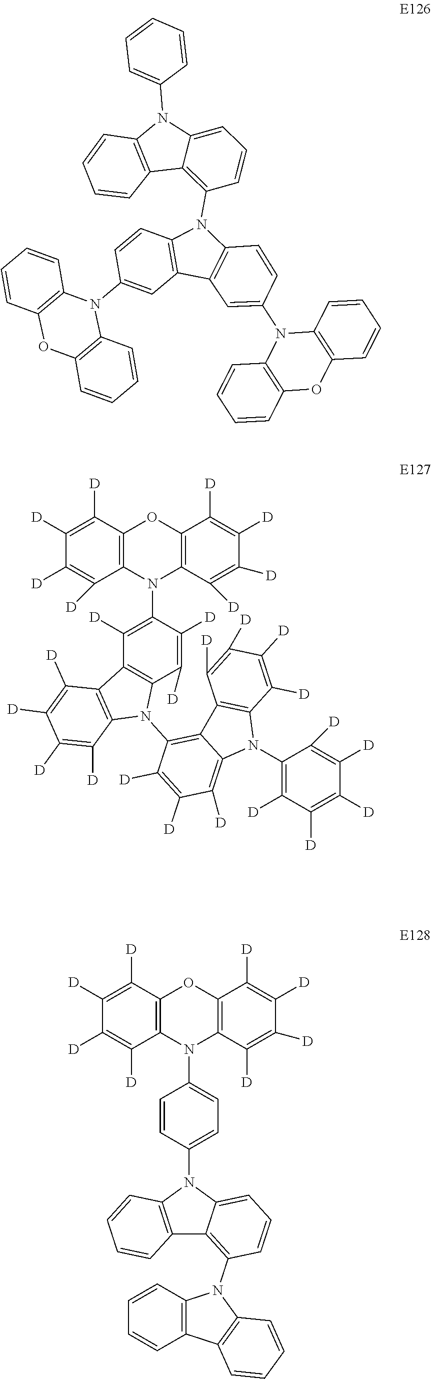

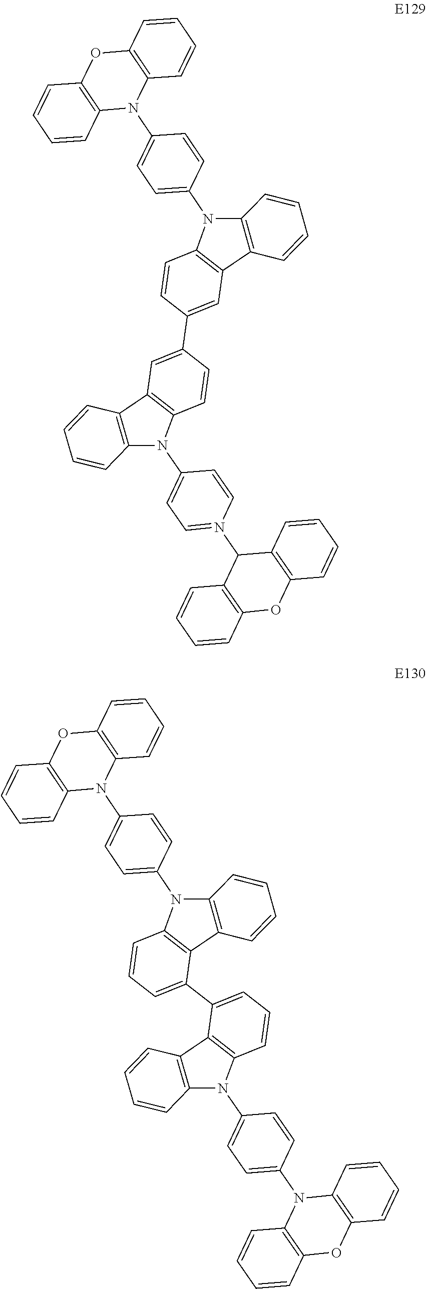

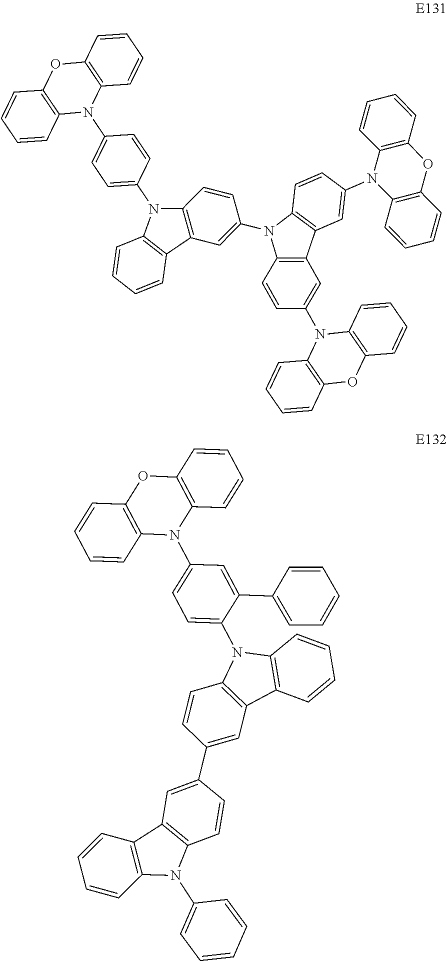

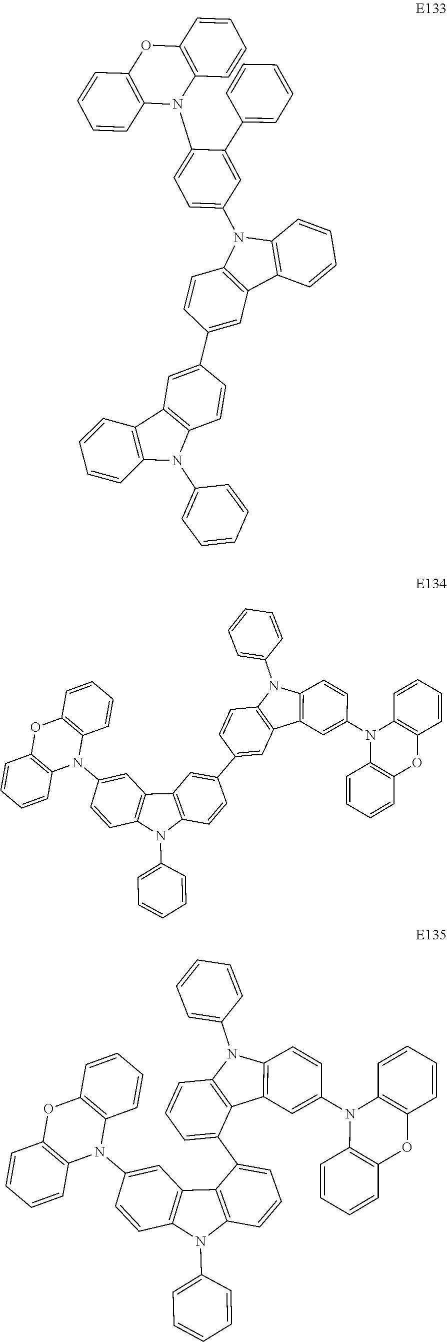

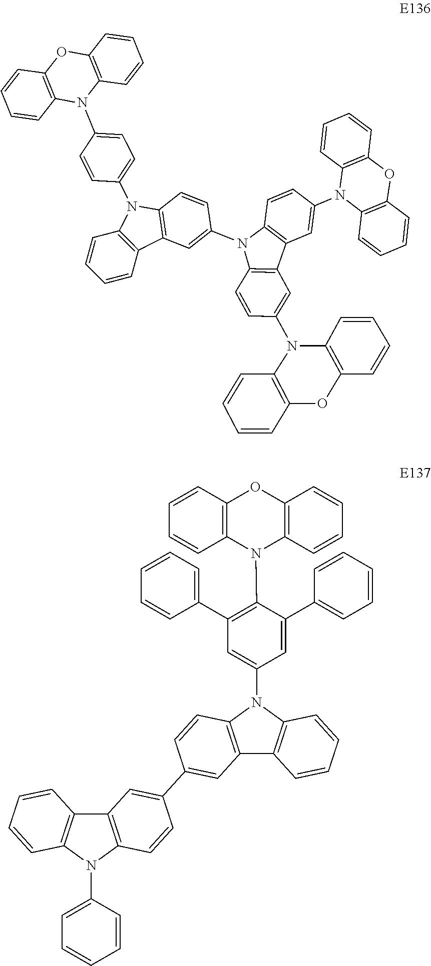

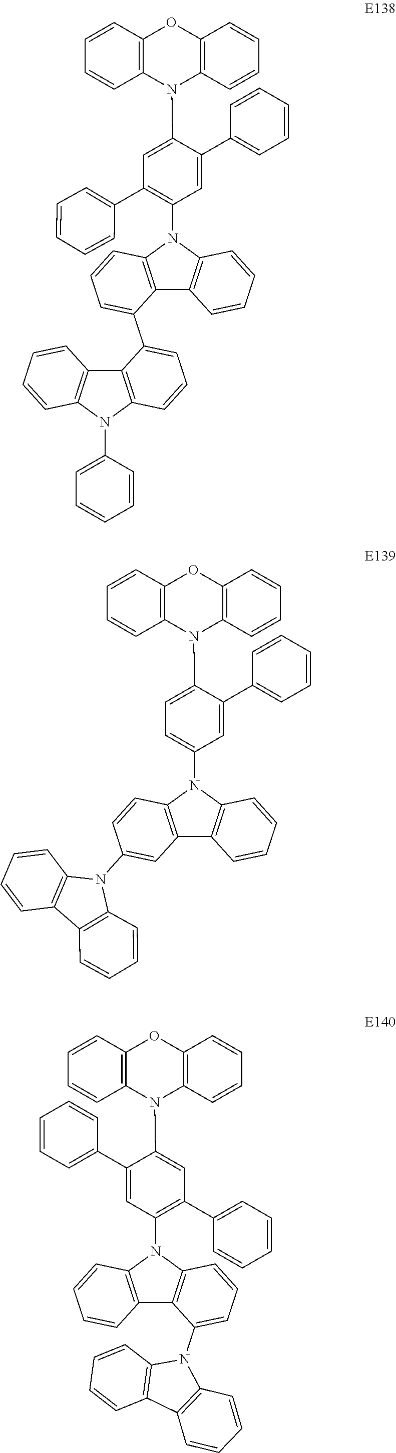

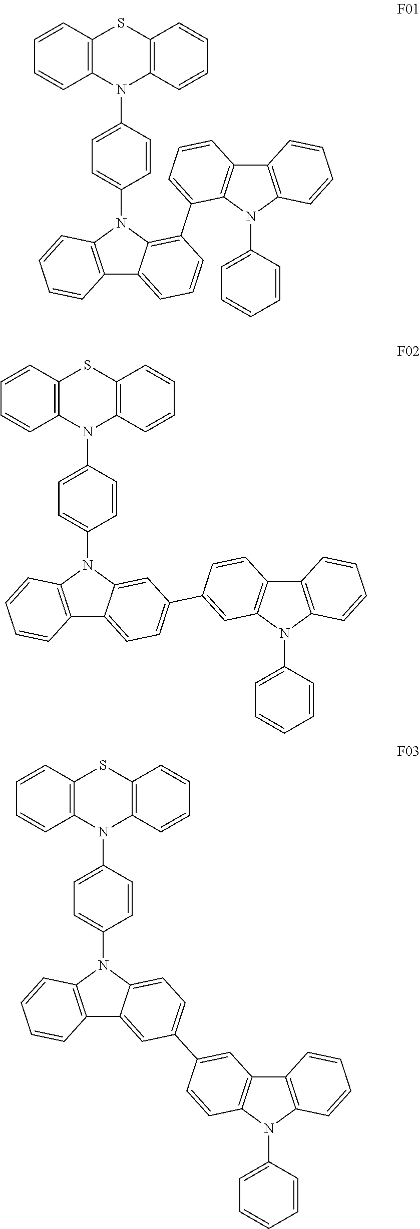

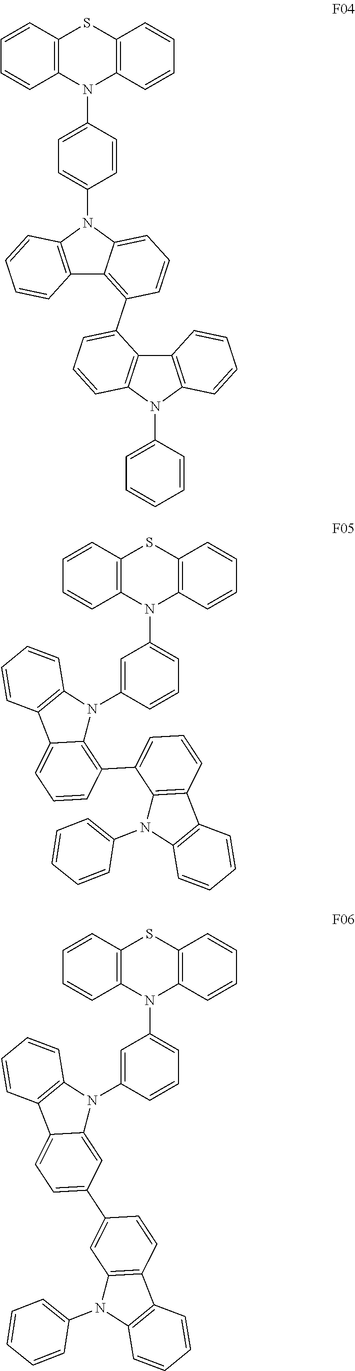

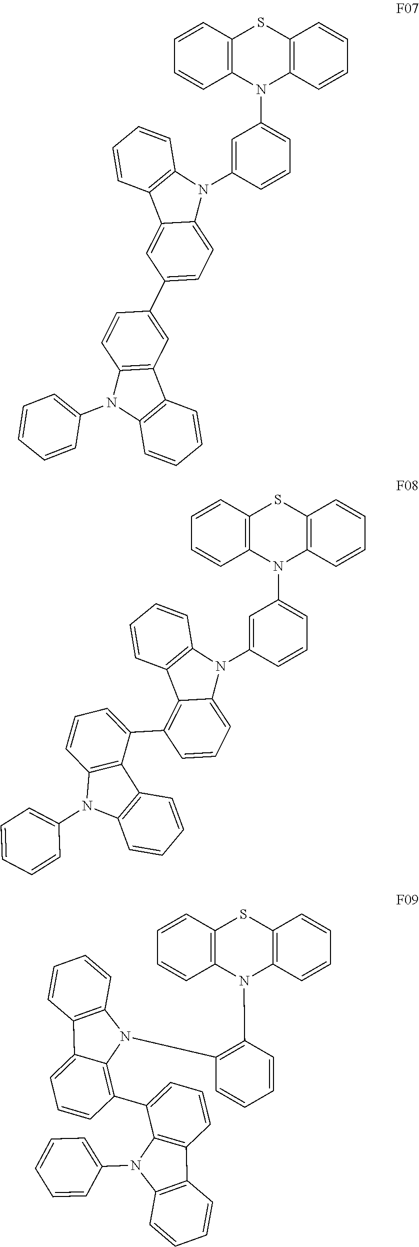

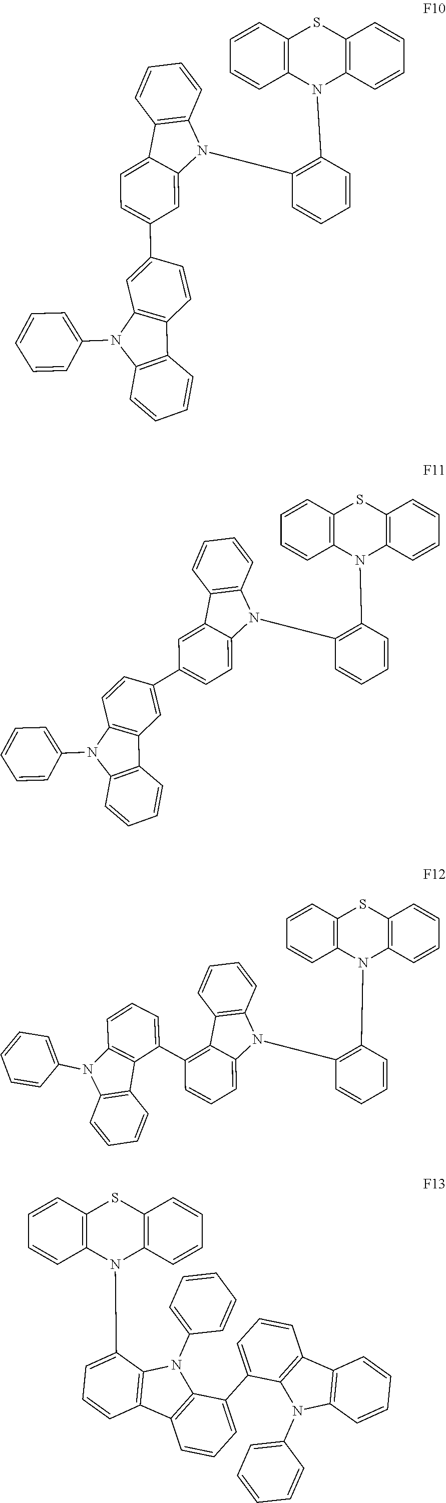

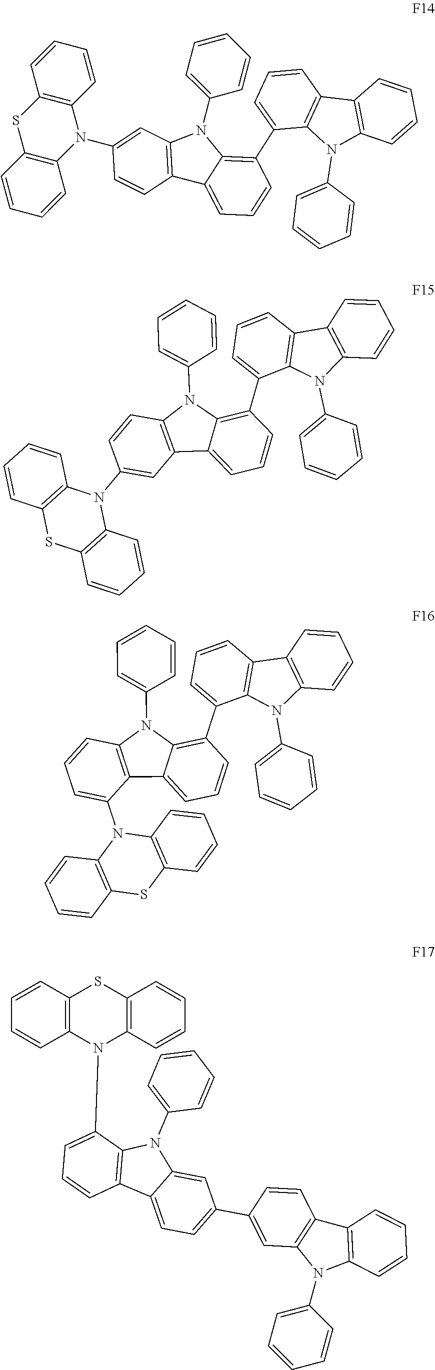

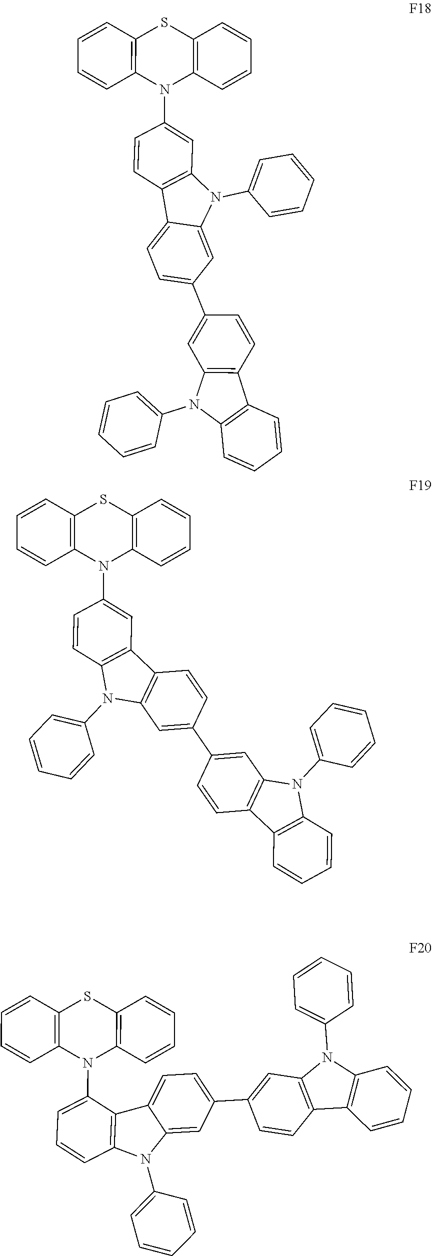

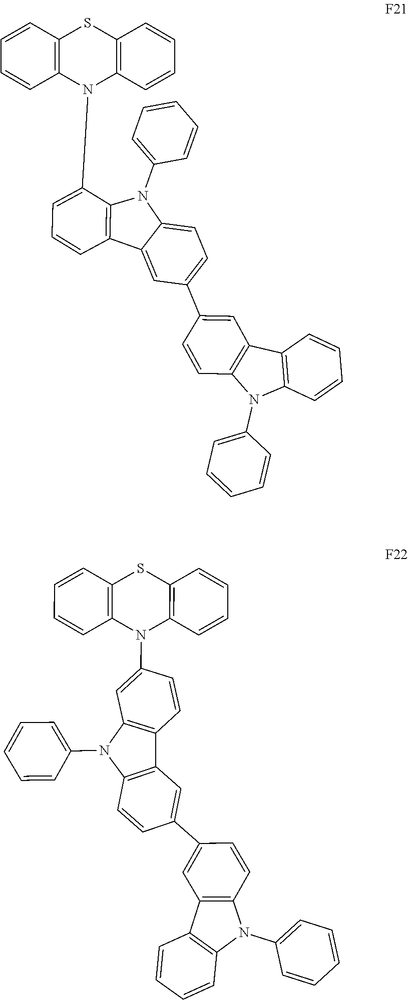

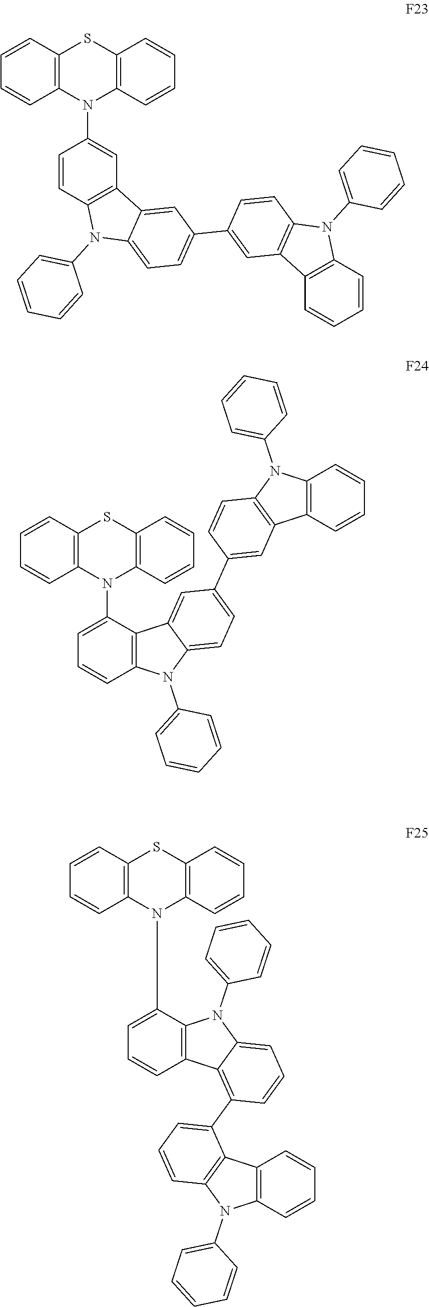

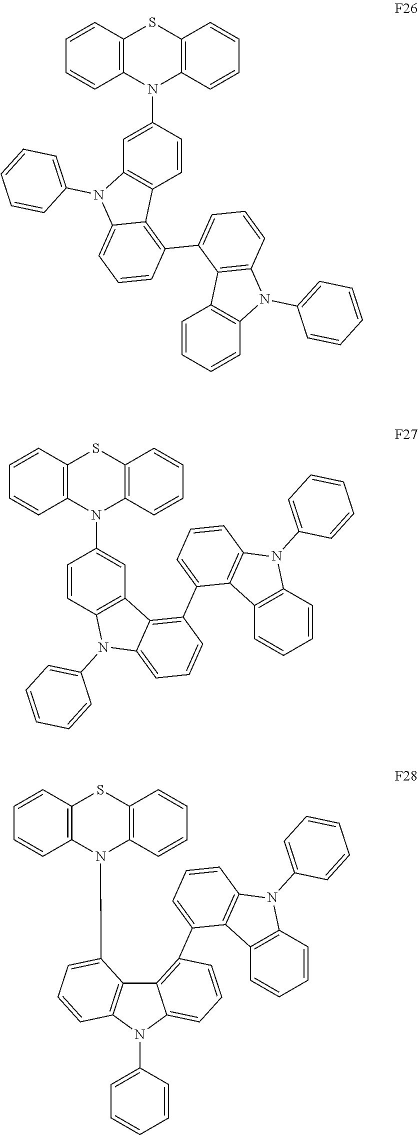

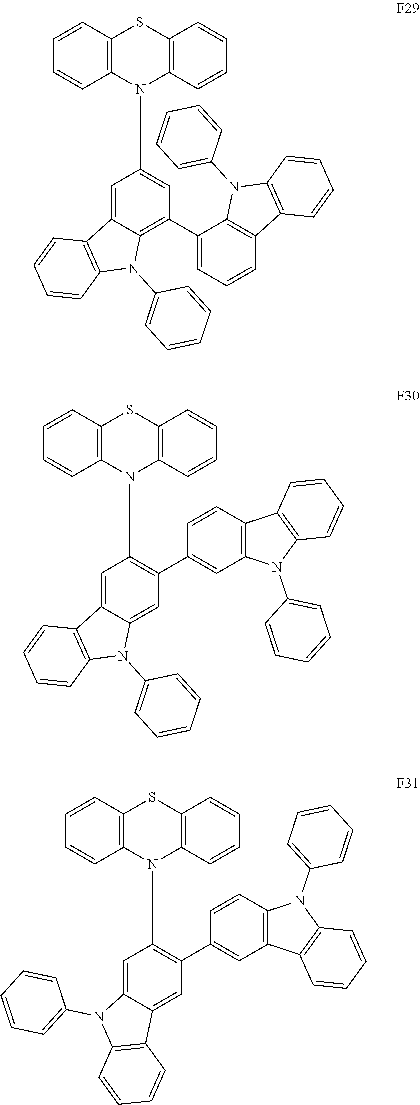

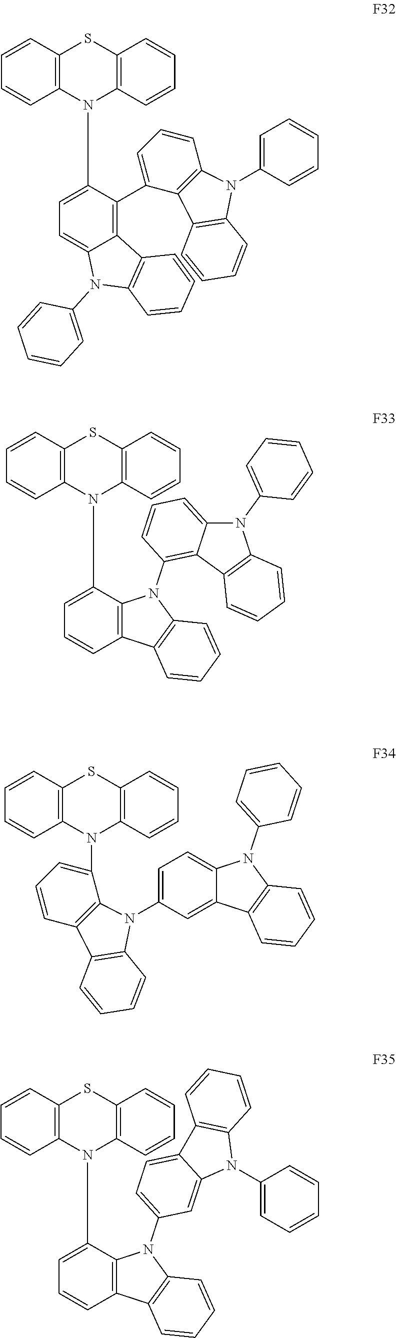

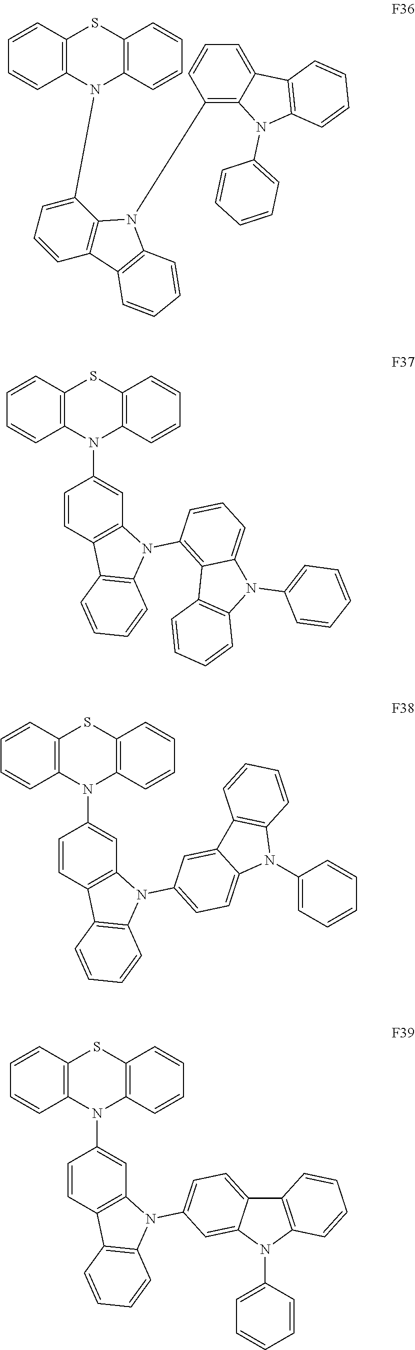

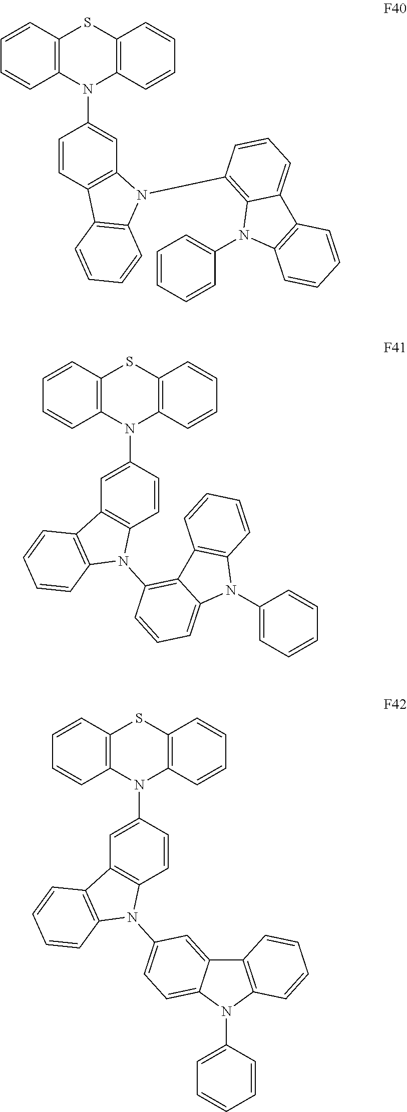

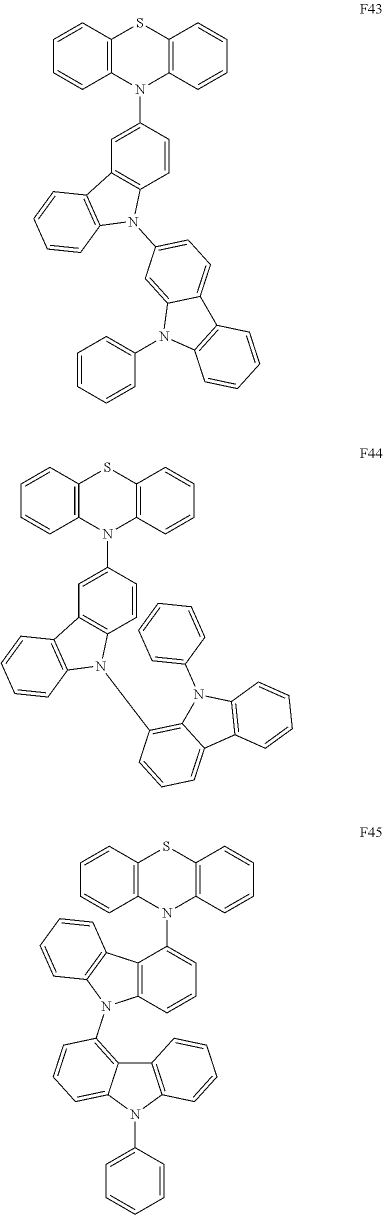

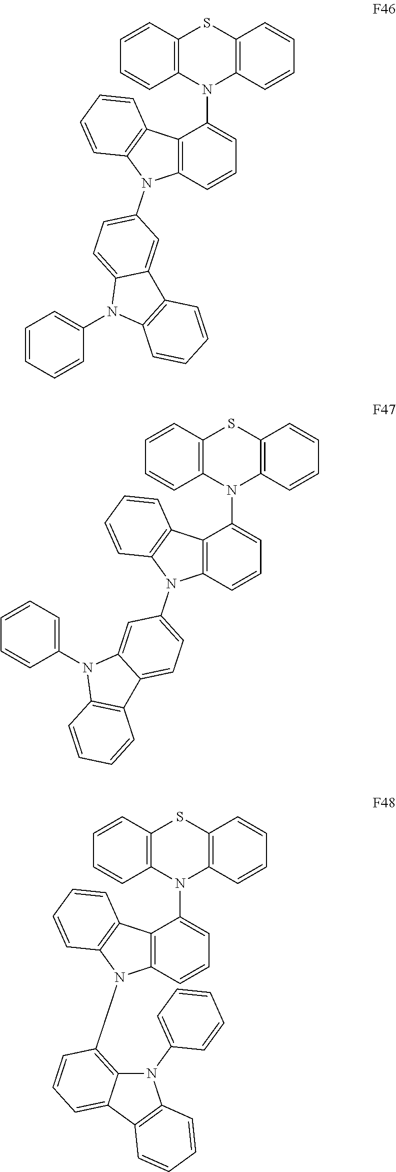

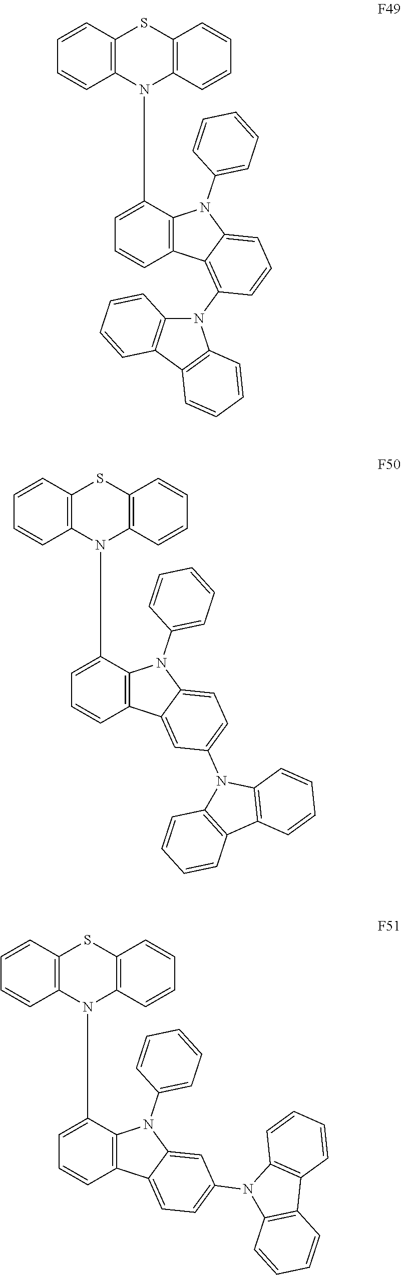

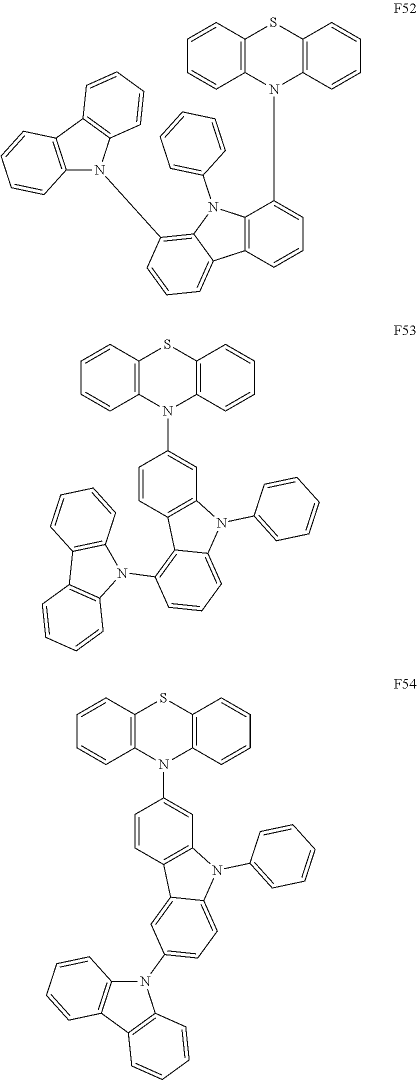

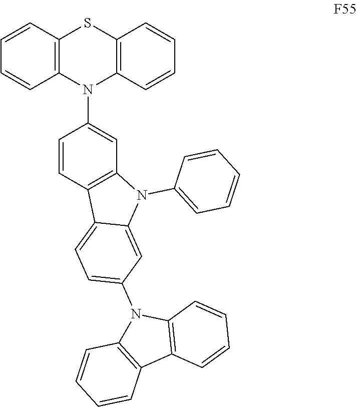

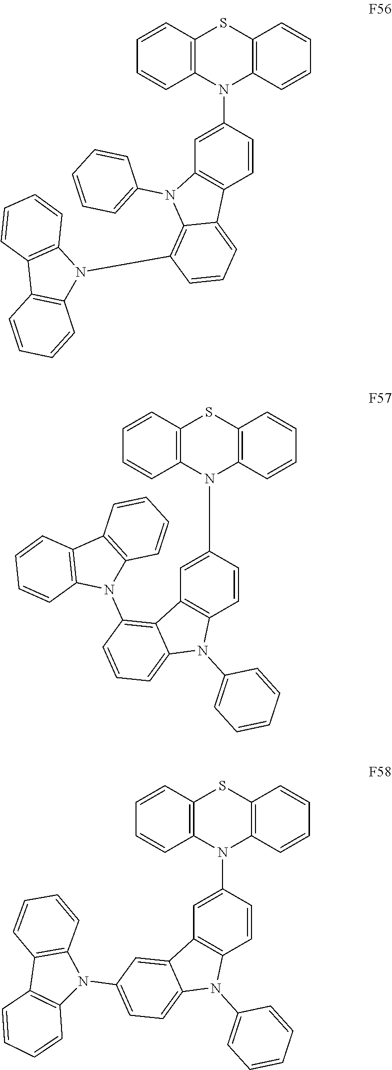

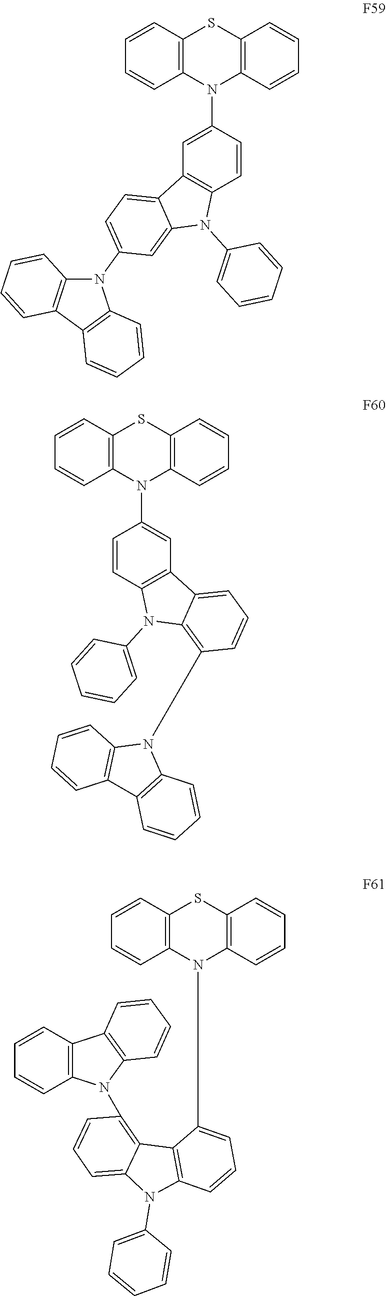

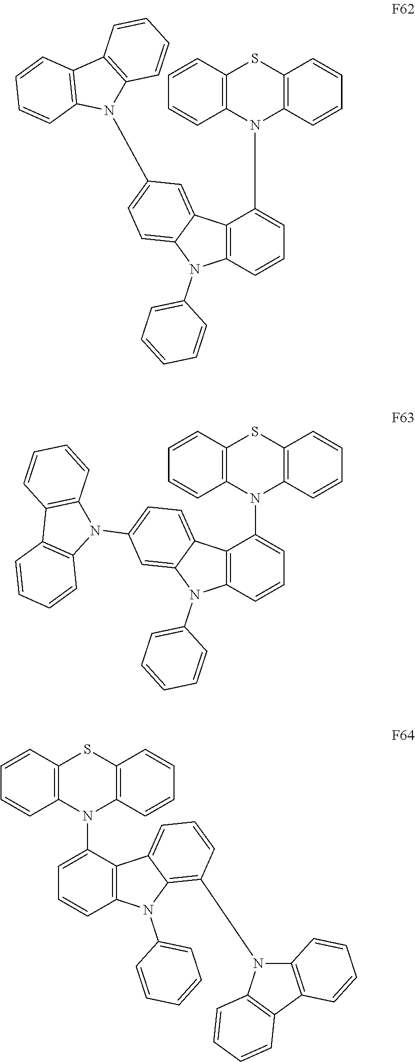

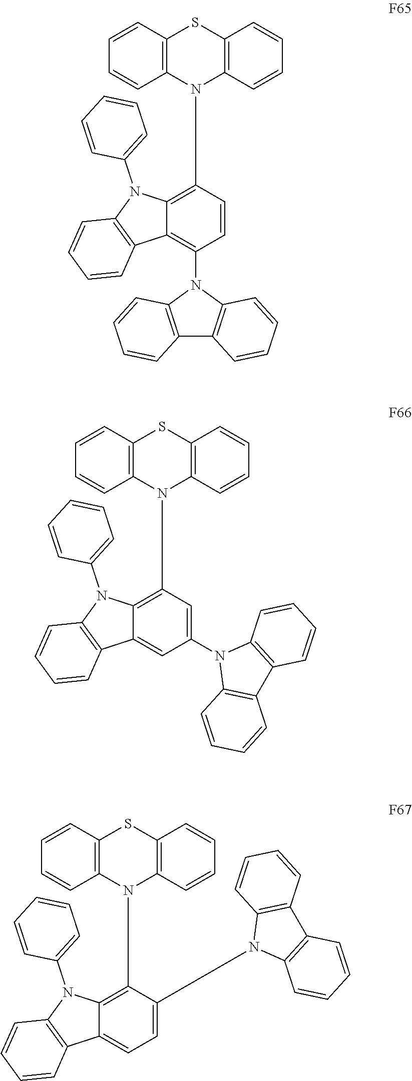

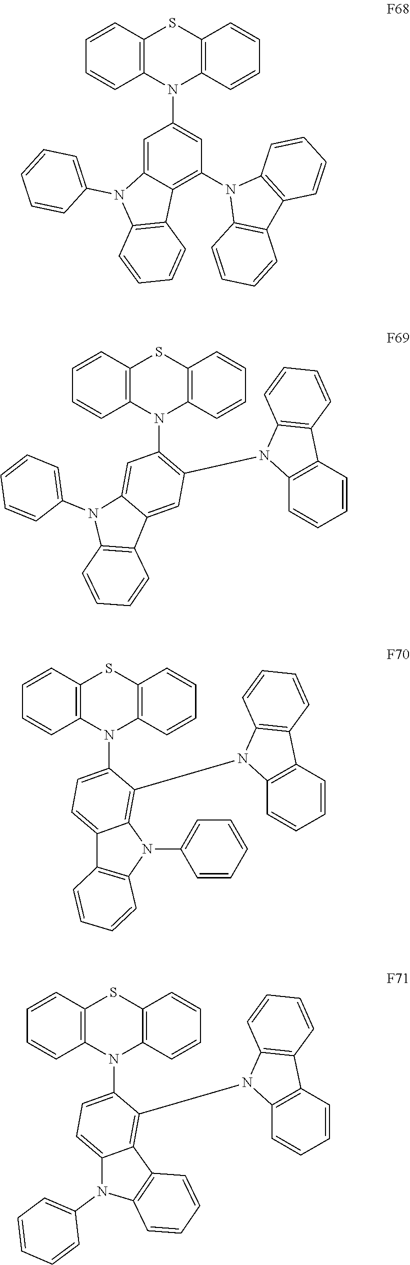

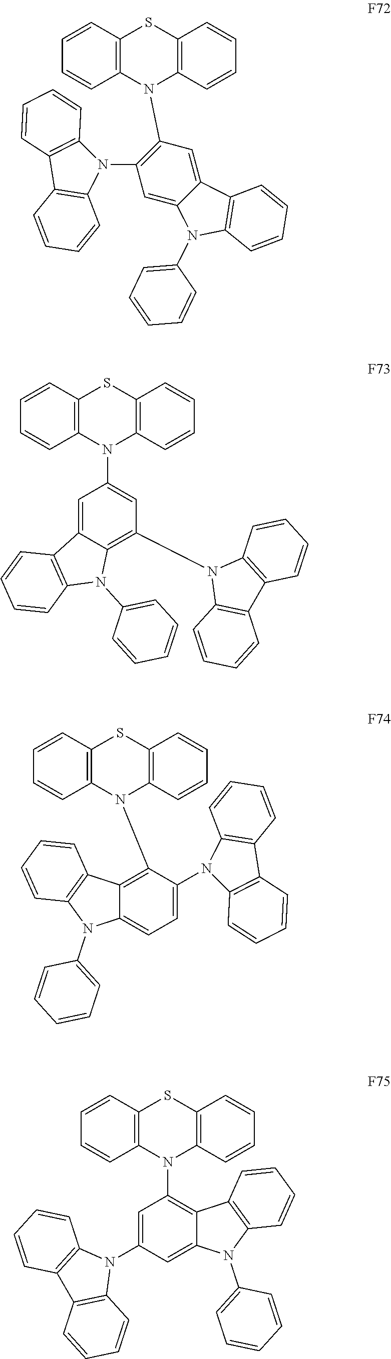

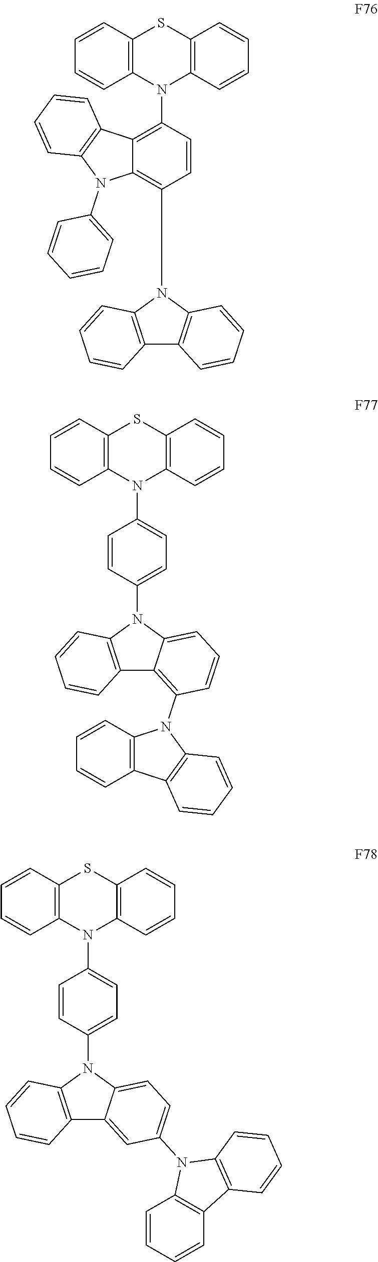

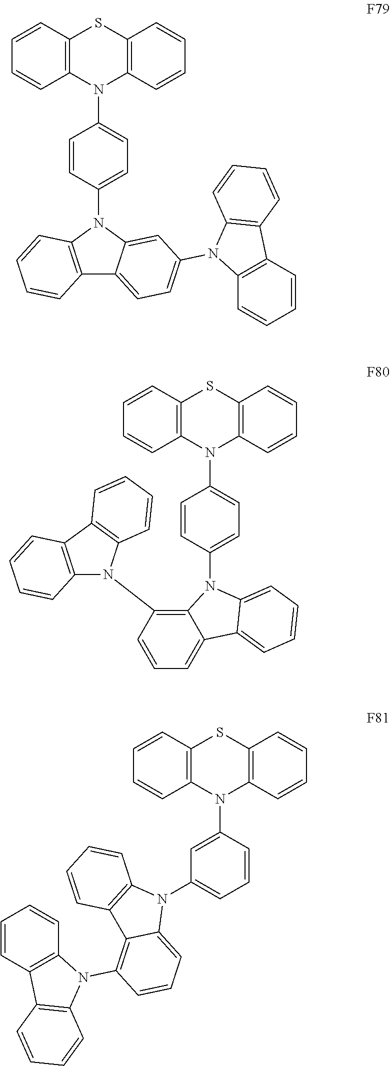

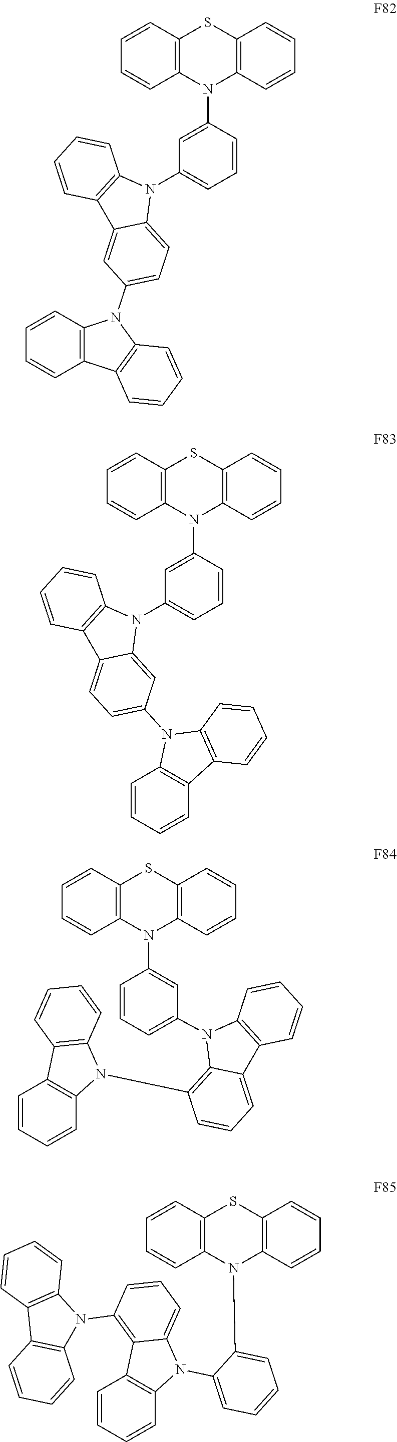

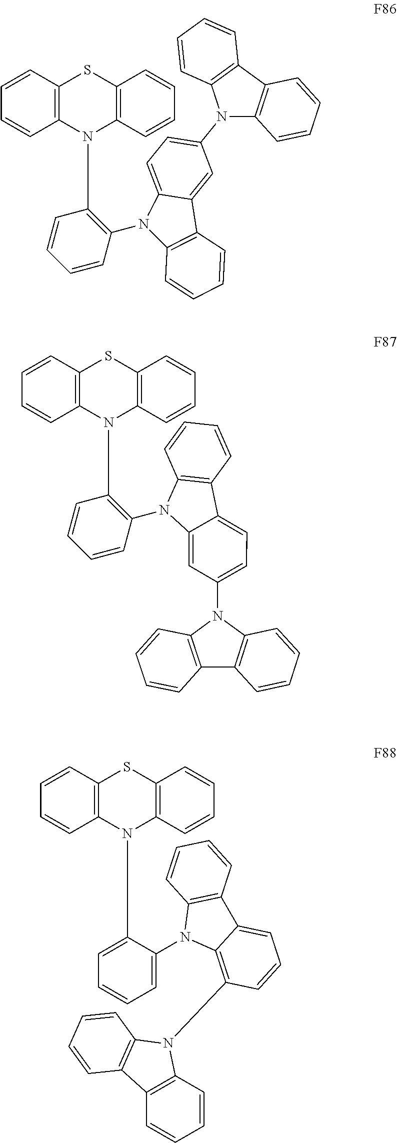

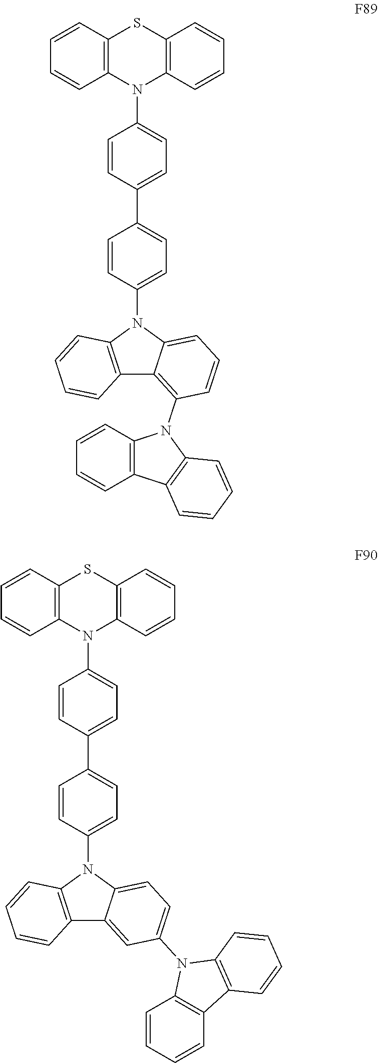

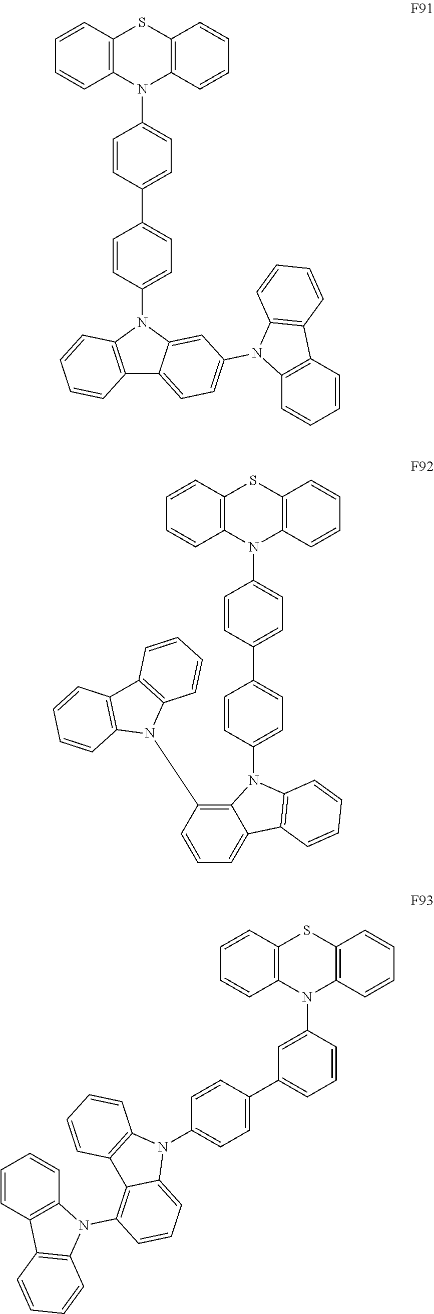

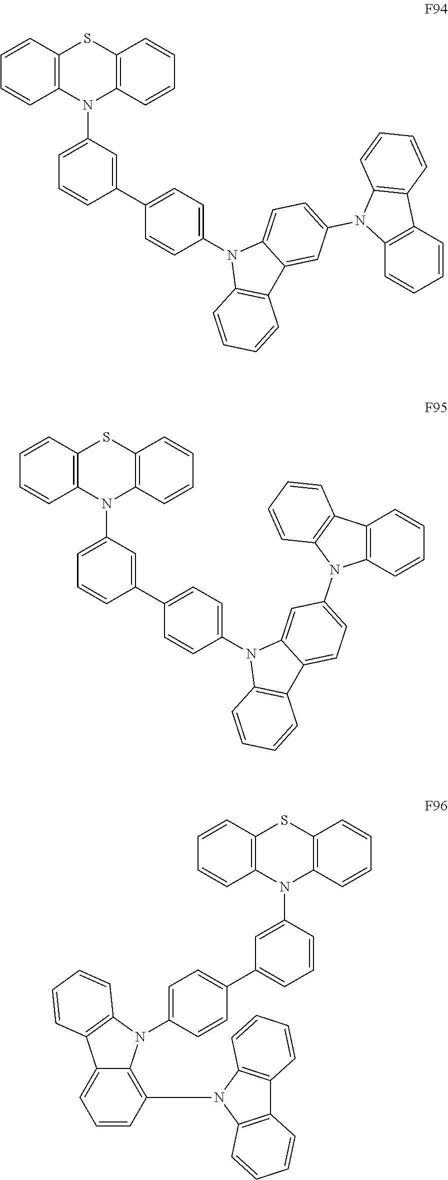

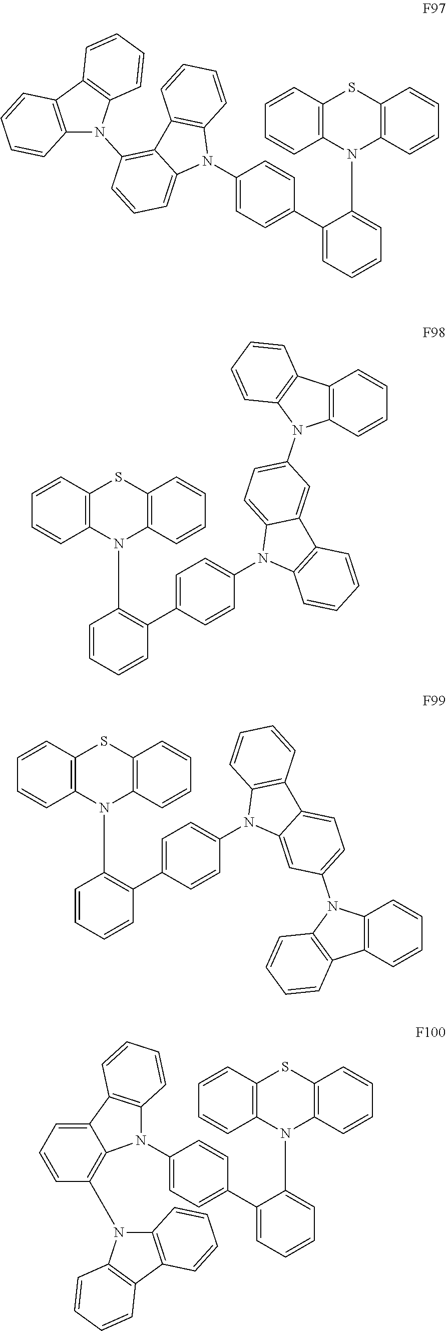

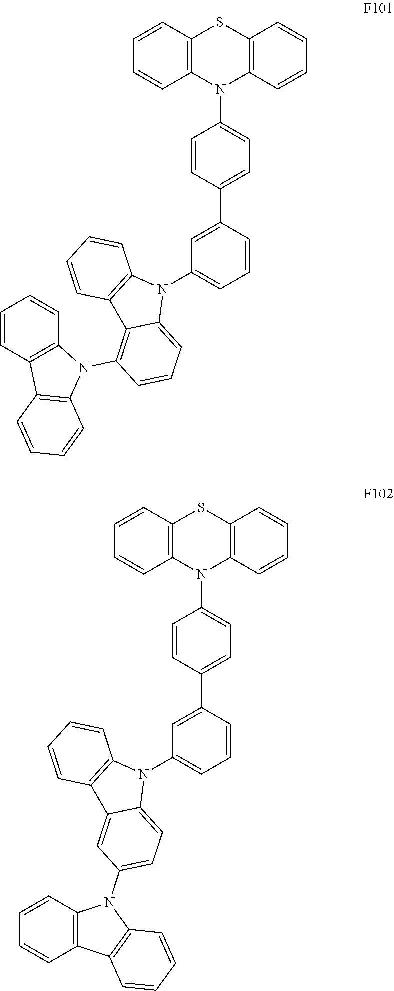

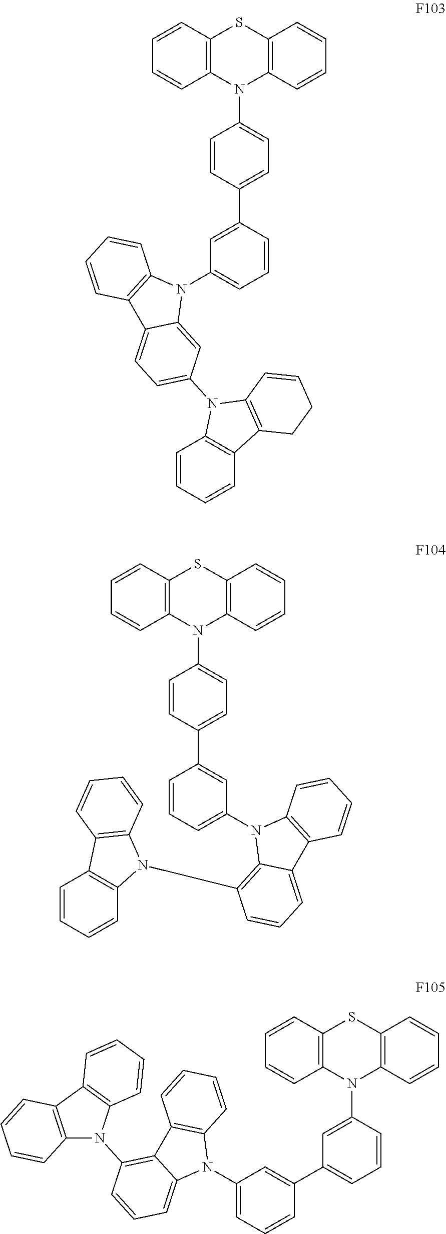

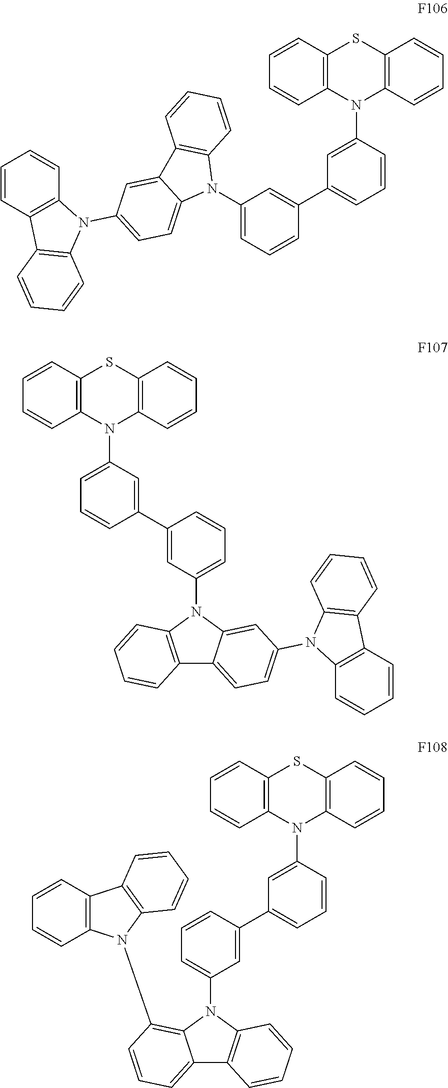

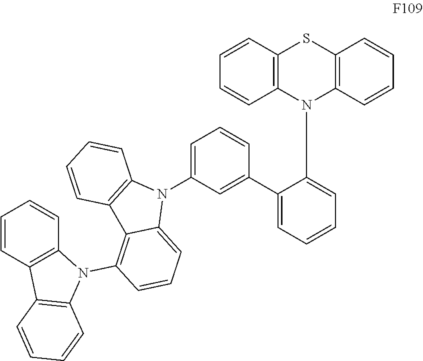

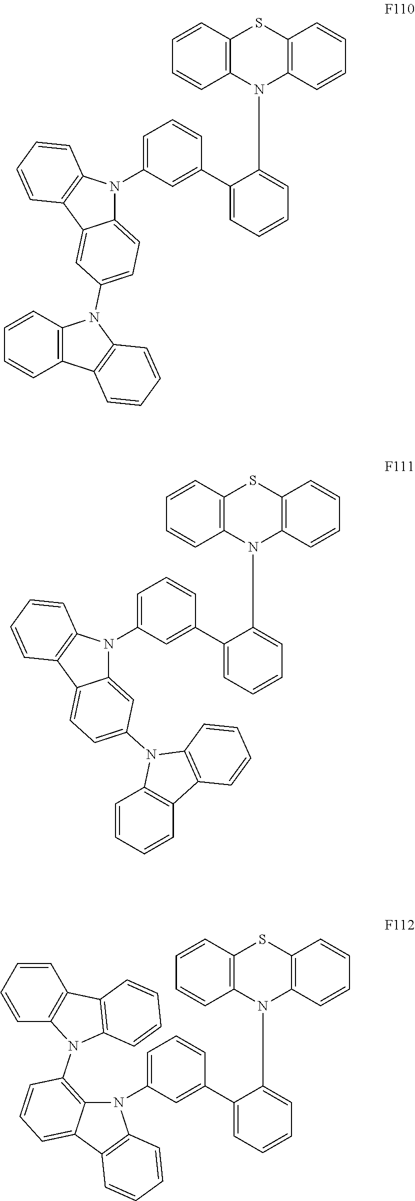

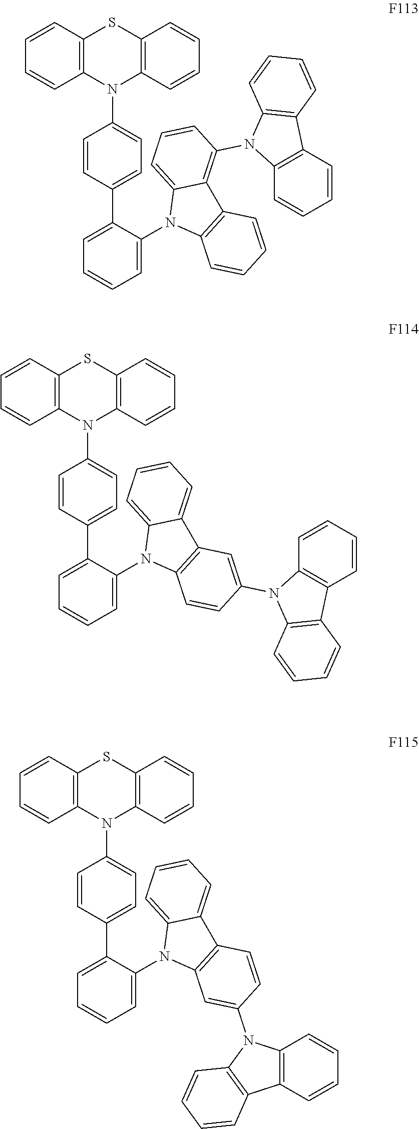

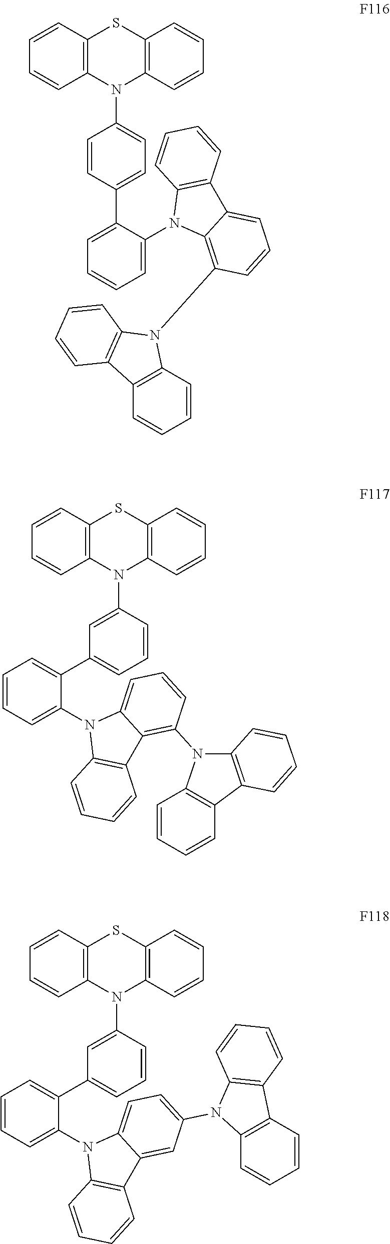

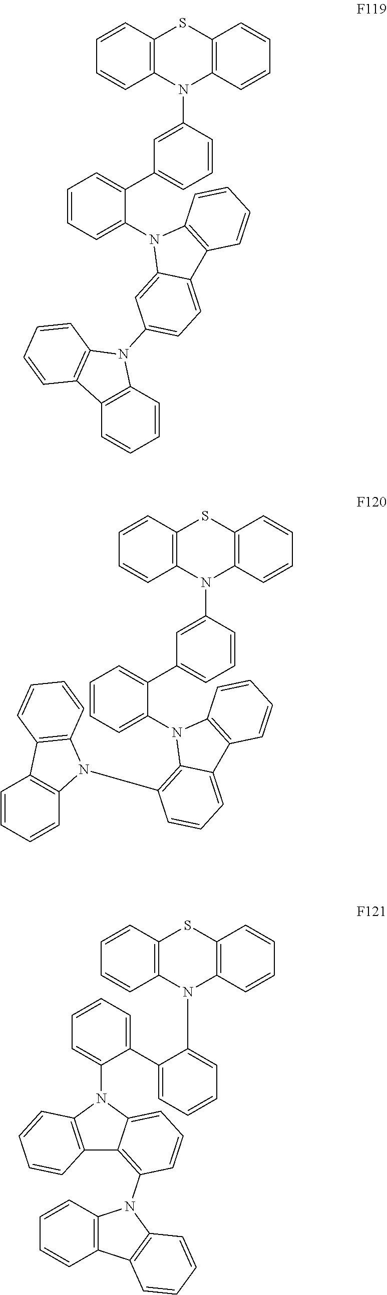

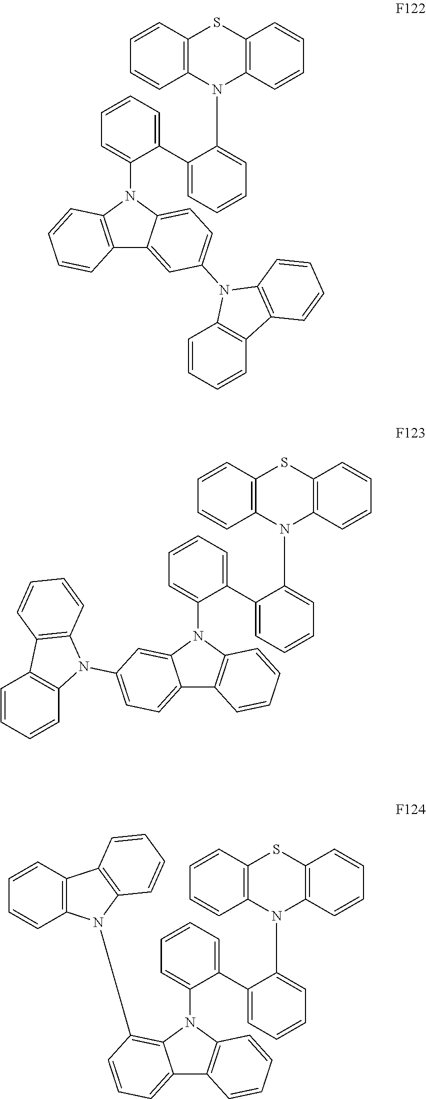

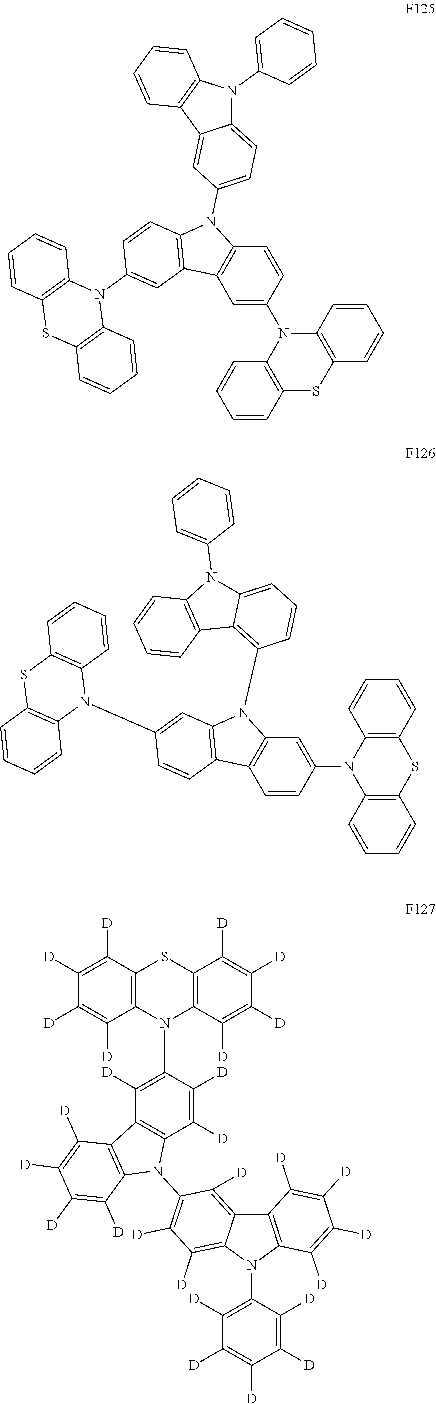

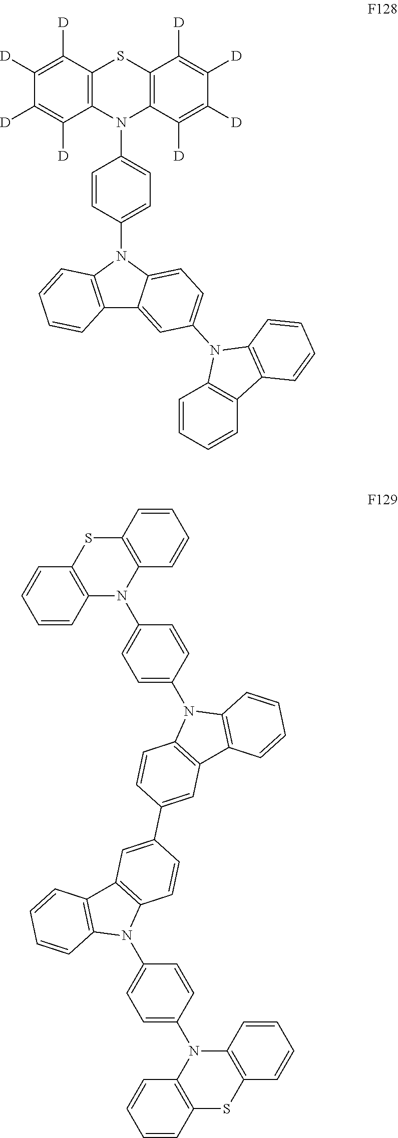

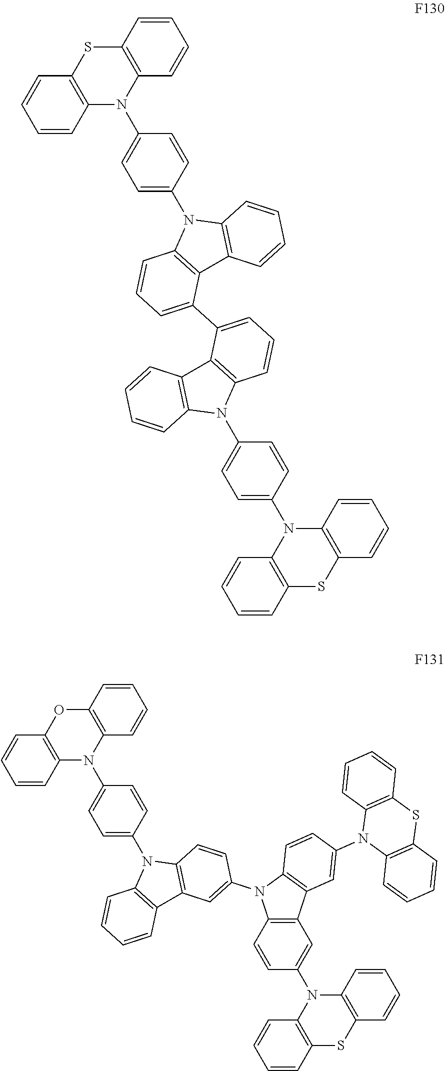

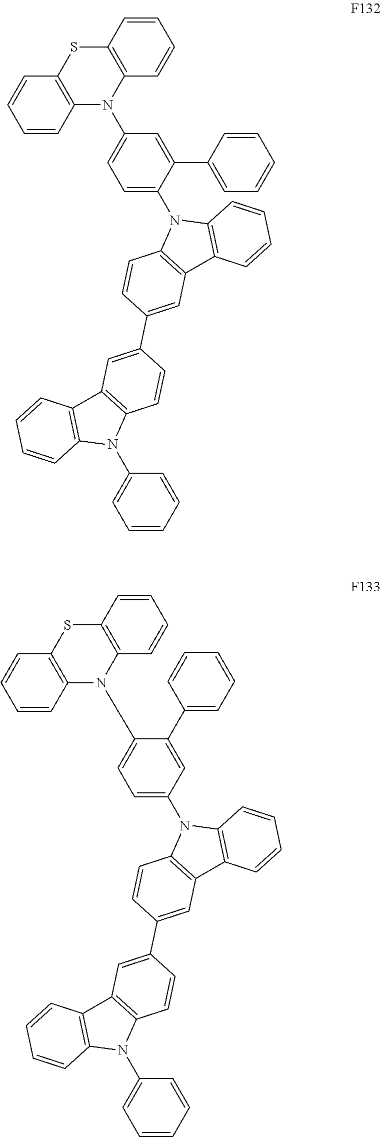

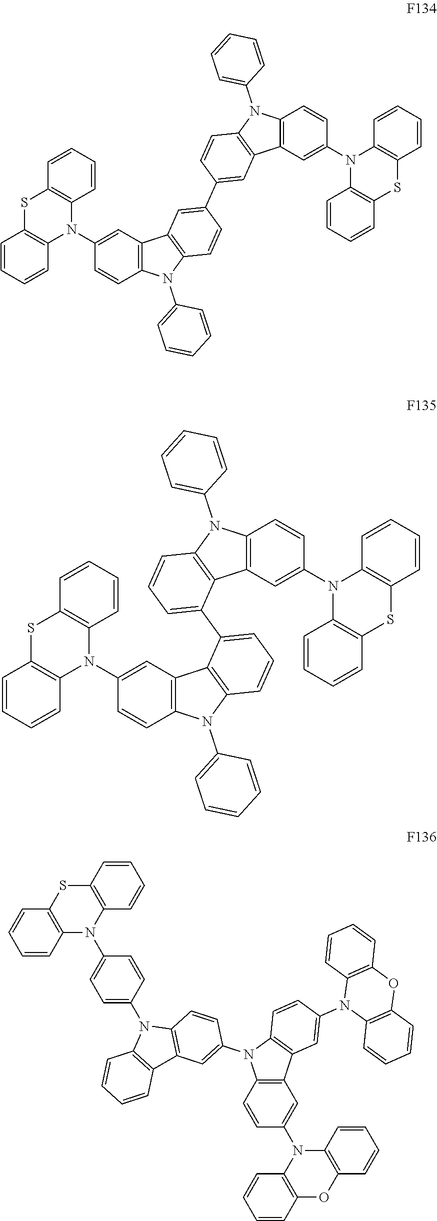

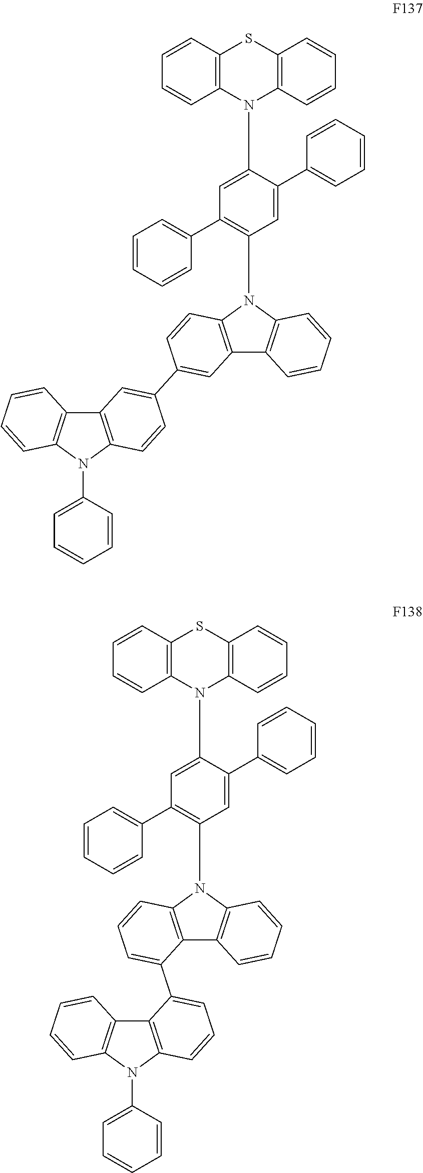

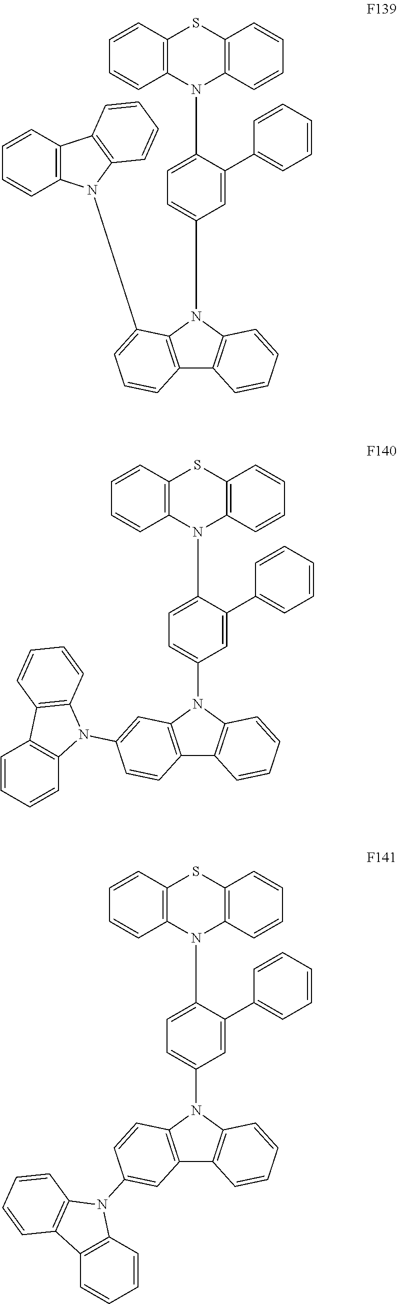

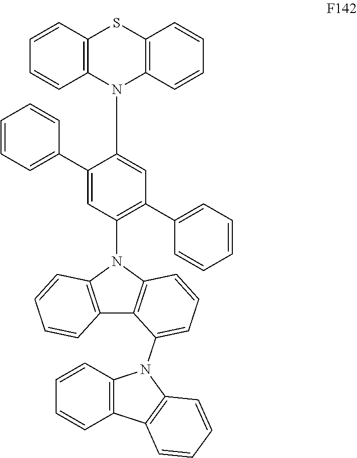

The compound of the present invention represented by the general formula (1) can be obtained by: synthesis by methods of various organic synthetic reactions established in the field of the organic synthetic chemistry including coupling reactions such as Suzuki coupling, Stille coupling, Grignard coupling, Ullmann coupling, Buchwald-Hartwig reaction, and Heck reaction, using commercially available reagents as raw materials; and then purification by using a known method such as recrystallization, column chromatography, and sublimation and purification. The method is not limited to this method.

The material for a photoelectric conversion device of the present invention preferably has an energy level of highest occupied molecular orbital (HOMO) obtained by structural optimization calculation with a density functional calculation B3LYP/6-31G(d) of −4.0 eV or lower, more preferably within a range of −6.0 eV to −4.3 eV, and further preferably within a range of −4.9 eV to −4.4 eV.

The material for a photoelectric conversion device of the present invention preferably has an energy level of lowest unoccupied molecular orbital (LUMO) obtained by structural optimization calculation with a density functional calculation B3LYP/6-31G(d) of −2.5 eV or higher, more preferably within a range of −2.0 eV to −0.5 eV, and further preferably within a range of −1.5 eV to −1.0 eV.

In the material for a photoelectric conversion device of the present invention, a difference (absolute value) between the HOMO energy level and the LUMO energy level is preferably within a range of 2.0 eV to 5.0 eV, more preferably within a range of 2.5 eV to 4.5 eV, and further preferably within a range of 3.5 eV to 4.5 eV.

The material for a photoelectric conversion device of the present invention preferably has a hole mobility of 1×10⁻⁶ cm²/Vs or more, preferably has a hole mobility of 1×10⁻⁵ cm²/Vs to 1 cm²/Vs, and more preferably has a hole mobility of 1.0×10⁻⁴ cm²/Vs to 1×10⁻² cm²/Vs. The hole mobility can be evaluated by known methods such as a method with a FET-type transistor device, a method with a time-of-flight method, and an SCLC method.

The material for a photoelectric conversion device of the present invention is preferably amorphous. The amorphousness can be confirmed by various methods, and can be confirmed by, for example, detecting no peak in an XRD method or by detecting no endothermic peak in a DSC method.

Next, a photoelectric conversion device for imaging using the material for a photoelectric conversion device of the present invention will be described, but a structure of the photoelectric conversion device for imaging of the present invention is not limited thereto. The description will be made with reference to the drawings.

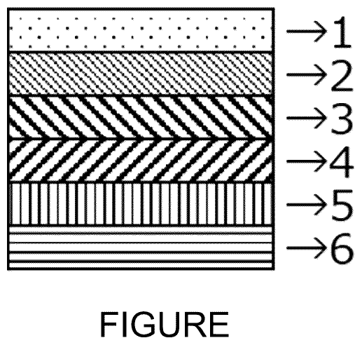

FIG. 1 is a sectional view schematically illustrating a structural example of the photoelectric conversion device for imaging of the present invention. In FIG. 1, 1 represents an electrode, 2 represents a hole blocking layer, 3 represents a photoelectric conversion layer, 4 represents an electron blocking layer, 5 represents an electrode, and 6 represents a substrate. Note that acceptable is structure in which configuration except for the substrate is inverted from that in FIG. 1, that is, 1 may represent the electrode, 2 may represent the electron blocking layer, 3 may represent the photoelectric conversion layer, 4 may represent the hole blocking layer, 5 may represent the electrode, and 6 may represent the substrate. The structure is not limited to one in FIG. 1, and a layer may be added or omitted as necessary.

### —Electrode—

An electrode used for the photoelectric conversion device for imaging using the material for a photoelectric conversion device for imaging of the present invention has a function of trapping a hole and an electron generated in the photoelectric conversion layer. A function to let light enter the photoelectric conversion layer is also required. Thus, at least one of two electrodes is desirably transparent or semi-transparent. A material used for the electrode is not particularly limited as long as it has conductivity, and examples thereof include: conductive transparent materials, such as ITO, IZO, SnO₂, ATO (antimony-doped tin oxide), Zno, AZO (Al-doped zinc oxide), GZO (gallium-doped zinc oxide), TiO₂, and FTO; metals, such as gold, silver, platinum, chromium, aluminum, iron, cobalt, nickel, and tungsten; inorganic conductive substances, such as copper iodide and copper sulfide; and conductive polymers, such as polythiophene, polypyrrole, and polyaniline. A plurality of these materials may be mixed to use as necessary. In addition, two or more layers thereof may be stacked.

### —Photoelectric Conversion Layer—

The photoelectric conversion layer is a layer in which a hole and an electrode are generated by charge separation of an exciton generated by the incident light. The photoelectric conversion layer may be formed with a single photoelectric converting material, or may be formed by combination with a P-type organic semiconductor material being a hole transport material and an N-type organic semiconductor material being an electron transport material. Two or more kinds of the P-type organic semiconductor may be used, and two or more kinds of the N-type organic semiconductor may be used. One or more kinds of these P-type organic semiconductor and/or N-type organic semiconductor desirably use a dye material having a function of absorbing light with a desired wavelength in the visible region. As the P-type organic semiconductor material being the hole transport material, the compound of the present invention represented by the general formula (1) can be used.

The P-type organic semiconductor material may be any material having a hole transportability. The material represented by the general formula (1) is preferably used, but another P-type organic semiconductor material may be used. In addition, two or more kinds of the material represented by the general formula (1) may be mixed to use. Furthermore, the compound represented by the general formula (1) and another P-type organic semiconductor material may be mixed to use.

The another P-type organic semiconductor material may be any material having the hole transportability, and for example, usable are: compounds having a fused polycyclic aromatic group such as naphthalene, anthracene, phenanthrene, pyrene, chrysene, naphthacene, triphenylene, perylene, fluoranthene, fluorene, and indene; compounds having a π-excess aromatic group such as a cyclopentadiene derivative, a furan derivative, a thiophene derivative, a pyrrole derivative, a benzofuran derivative, a dibenzothiophene derivative, a dinaphthothienothiophene derivative, an indole derivative, a pyrazoline derivative, a dibenzofuran derivative, a dibenzothiophene derivative, a carbazole derivative, and an indolocarbazole derivative; an aromatic amine derivative, a styrylamine derivative, a benzidine derivative, a porphyrin derivative, a phthalocyanine derivative, and a quinacridone derivative.

In addition, examples of a polymer P-type organic semiconductor material include a polyphenylene-vinylene derivative, a polyparaphenylene derivative, a polyfluorene derivative, a polyvinylcarbazole derivative, and a polythiophene derivative. Two or more kinds selected from the compound represented by the general formula (1), the P-type organic semiconductor material, and the polymer P-type organic semiconductor material may be mixed to use.

The N-type organic semiconductor material may be any material having the electron transportability, and examples thereof include naphthalenetetracarboxylic diimide and perylenetetracarboxylic diimide, fullerenes, and azole derivatives such as imidazole, thiazole, thiadiazole, oxazole, oxadiazole, and triazole. Two or more kinds selected from the N-type organic semiconductor materials may be mixed to use.

### —Electron Blocking Layer—

The electron blocking layer is provided in order to inhibit a dark current generated by injecting an electron from one electrode into the photoelectric conversion layer when a bias voltage is applied between the two electrodes. The electron blocking layer also has a function of hole transportation for transporting a hole generated by charge separation in the photoelectric conversion layer toward the electrode. A single layer or multiple layers of the electron blocking layer can be disposed as necessary. For the electron blocking layer, a P-type organic semiconductor material being the hole transport material can be used. The P-type organic semiconductor material may be any material having the hole transportability. Although the compound represented by the general formula (1) is preferably used, another P-type organic semiconductor material may be used. The compound represented by the general formula (1) and another P-type organic semiconductor material may be mixed to use. The other P-type organic semiconductor material may be any material having the hole transportability, and for example, usable are: compounds having a fused polycyclic aromatic group such as naphthalene, anthracene, phenanthrene, pyrene, chrysene, naphthacene, triphenylene, perylene, fluoranthene, fluorene, and indene; compounds having a π-excess aromatic group such as a cyclopentadiene derivative, a furan derivative, a thiophene derivative, a pyrrole derivative, a benzofuran derivative, a dibenzothiophene derivative, a dinaphthothienothiophene derivative, an indole derivative, a pyrazoline derivative, a dibenzofuran derivative, a dibenzothiophene derivative, and a carbazole derivative; an aromatic amine derivative, a styrylamine derivative, a benzidine derivative, a porphyrin derivative, a phthalocyanine derivative, and a quinacridone derivative.

In addition, examples of a polymer P-type organic semiconductor material include a polyphenylene-vinylene derivative, a polyparaphenylene derivative, a polyfluorene derivative, a polyvinylcarbazole derivative, and a polythiophene derivative. Two or more kinds selected from the compound of the present invention represented by the general formula (1), the P-type organic semiconductor material, and the polymer P-type organic semiconductor material may be mixed to use.

### —Hole Blocking Layer—

The hole blocking layer is provided in order to inhibit a dark current generated by injecting a hole from one electrode into the photoelectric conversion layer when a bias voltage is applied between the two electrodes. The hole blocking layer also has a function of electron transportation for transporting an electron generated by charge separation in the photoelectric conversion layer toward the electrode. A single layer or multiple layers of the hole blocking layer can be disposed as necessary. For the hole blocking layer, the N-type organic semiconductor material having the electron transportability can be used.

The N-type organic semiconductor material may be any material having the electron transportability, and examples thereof include: polycyclic aromatic multivalent carboxylic anhydride or imidized products thereof, such as naphthalenetetracarboxylic diimide and perylenetetracarboxylic diimide; fullerenes, such as C60 and C70; azole derivatives, such as imidazole, thiazole, thiadiazole, oxazole, oxadiazole, and triazole; a tris(8-quinolinolate)aluminum (III) derivative, a phosphine oxide derivative, a nitro-substituted fluorene derivative, a diphenylquinone derivative, a thiopyran dioxide derivative, a carbodiimide derivative, a fluorenylidene methane derivative, an anthraquinodimethane derivative and an anthrone derivative, a bipyridine derivative, a quinoline derivative, and an indolocarbazole derivative. Two or more kinds of these N-type organic semiconductor materials may be mixed to use.

As mentioned above, hydrogen in the material of the present invention may be deuterium. That is, a part or all of hydrogens on the aromatic rings in the general formula (1) or the general formulae (2) to (8), and hydrogen of the substituents may be deuterium.

Furthermore, a part or all of hydrogens in a compound used as the N-type organic semiconductor material and the P-type organic semiconductor material may be deuterium.

A method for producing a film of each layer in producing the photoelectric conversion device for imaging of the present invention is not particularly limited. The photoelectric conversion device may be produced by any one of dry process and wet process.

The organic layer containing the material for a photoelectric conversion device of the present invention may be a plurality of the layers as necessary.

### EXAMPLES

Hereinafter, the present invention will be described in more detail with Examples, but the present invention is not limited to these Examples.

### Calculation Example

### Calculation of HOMO and LUMO

Calculated were HOMO, LUMO and a difference in energy of HOMO and LUMO of the above compounds A4, C13, D61, E42, E45, E58, E126, F42, and F125. The calculation was performed by using a density functional theory (DFT), using Gaussian as a calculation program, and with structural optimization calculation of a density functional calculation B3LYP/6-31G(d). Table 1 shows the results. It can be mentioned that any of the materials for the photoelectric conversion device of the present invention has preferable HOMO and LUMO values.

As comparative compounds H1, H2, and H3, HOMO, LUMO, and the difference in energy of HOMO and LUMO were calculated in the same manner. Table 1 shows the results.

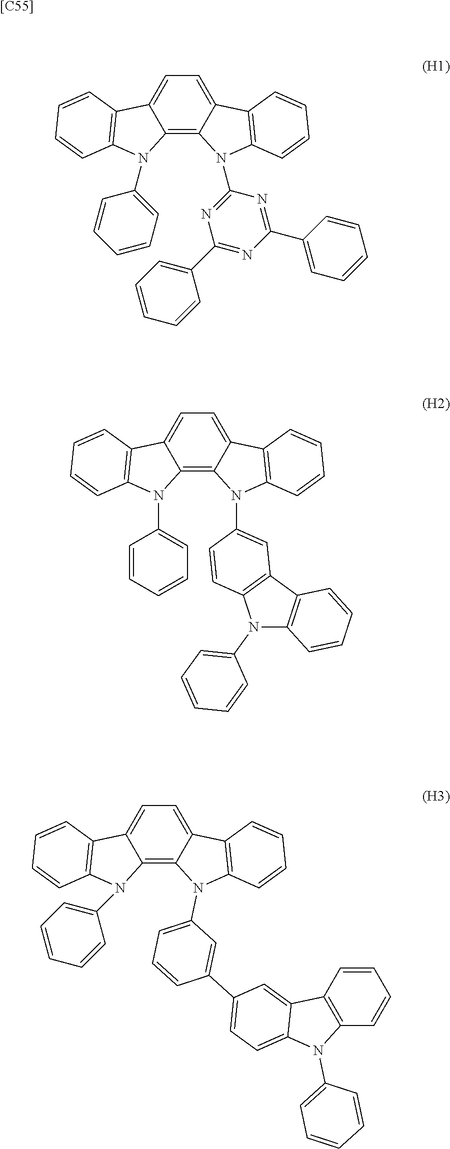

TABLE 1

| Compound | HOMO[eV] | LUMO[eV] | Energy difference [eV] |
|---|---|---|---|
| A4 | −4.9 | −0.7 | 4.2 |
| C13 | −4.8 | −1.1 | 3.7 |
| D61 | −4.2 | −1.0 | 3.2 |
| E42 | −4.4 | −1.1 | 3.3 |
| E45 | −4.5 | −1.0 | 3.5 |
| E58 | −4.6 | −1.2 | 3.3 |
| E126 | −4.5 | −1.2 | 3.3 |
| F42 | −4.7 | −1.1 | 3.7 |
| F125 | −4.9 | −1.2 | 3.7 |
| H1 | −5.3 | −1.9 | 3.4 |
| H2 | −5.0 | −0.9 | 4.1 |
| H3 | −5.1 | −0.8 | 4.2 |

A synthesis example of the compound E42 will be described below as a representative example. The other compounds were also synthesized by a similar method.

### Synthesis Example 1 (Synthesis of Intermediate R3)

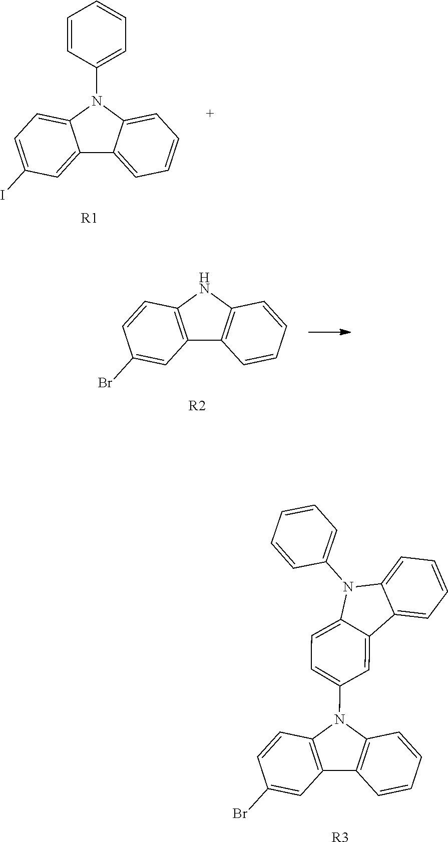

Into a three-necked 500-ml flask with degassed and nitrogen-replenished, R1 (19.0 mmol), R2 (19.9 mmol), copper iodide (0.4 mmol), and tripotassium phosphate (56.9 mmol) were added, 70 ml of dehydrated dioxane was added thereinto, and then the mixture was stirred at 120 °C for 3 hours. The mixture was cooled to a room temperature, then an inorganic substance was removed by filtration, 200 ml of water and 200 ml of dichloromethane were added into the filtrate and transferred to a separatory funnel, and separation into an organic layer and an aqueous layer was performed. The organic layer was washed three times with 200 ml of water, the obtained organic layer was dehydrated with magnesium sulfate, and then concentrated under a reduced pressure. The obtained residue was purified by column chromatography to obtain R3 (pale yellow solid). The yield quantity was 7.5 g, and the yield rate was 81.3%.

### Synthesis Example 2 (Synthesis of E42)

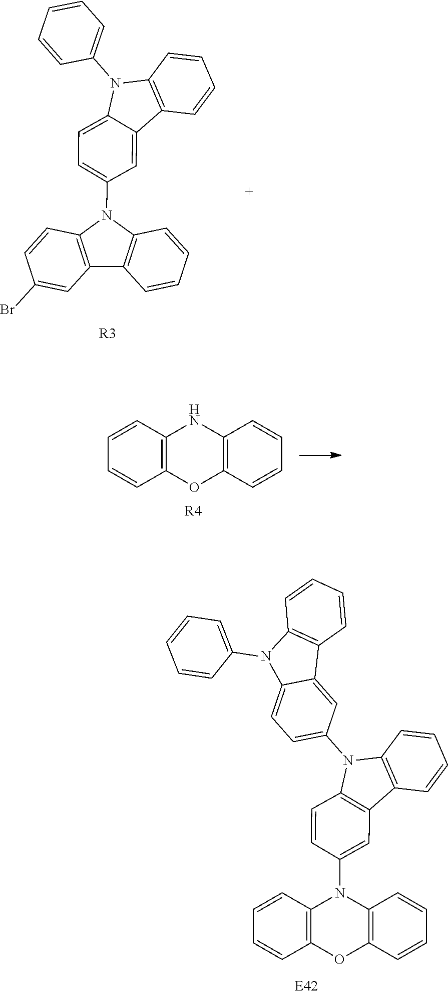

Into a three-necked 300-ml flask with degassed and nitrogen-replenished, R3 (10.3 mmol) synthesized in Synthesis Example 1, R4 (13.3 mmol), trisdibenzylidene acetone dipalladium (0.5 mmol), tri-tert-butylphosphine (2.1 mmol), sodium tert-butoxide (30.8 mmol), and 50 ml of dehydrated xylene were fed, and the mixture was stirred at 120 °C for 2 hours. The mixture was cooled to a room temperature, then an inorganic substance was removed by filtration, 200 ml of water and 200 ml of dichloromethane were added into the filtrate and transferred to a separatory funnel, and separation into an organic layer and an aqueous layer was performed. The organic layer was washed three times with 200 ml of water, the obtained organic layer was dehydrated with magnesium sulfate, and then concentrated under a reduced pressure. The obtained residue was purified by column chromatography to obtain E42 (pale yellow solid). The yield quantity was 4.2 g, and the yield rate was 69.4%. The obtained solid was evaluated by an XRD method but no peak was detected. Thus, this compound was found to be amorphous.

### Example of Physical Properties Evaluation

On a glass substrate on which a transparent electrode composed of ITO with 110 nm in film thickness was formed, the compound E42 was produced to a film as an organic layer by a vacuum deposition method under a condition that a film thickness was approximately 3 μm. Subsequently, charge mobility was measured by a time-of-flight method using a device in which aluminum (Al) was formed with 70 nm in thickness as an electrode. As a result, the hole mobility was 1.4×10⁻⁴ cm²/Vs.

The hole mobilities were evaluated in the same procedure as above except that A04, C13, E45, E126, F125, H1, H2, or H3 was used instead of the compound E42. Table 2 shows the results.

TABLE 2

| Compound | hole mobility [cm²/Vs] |
|---|---|
| A04 | 5.9 × 10⁻⁴ |
| C13 | 7.7 × 10⁻⁵ |
| E42 | 1.4 × 10⁻⁴ |
| E45 | 1.0 × 10⁻⁴ |
| E126 | 1.9 × 10⁻⁴ |
| F125 | 2.3 × 10⁻⁴ |
| H1 | 4.0 × 10⁻⁵ |
| H2 | 3.0 × 10⁻⁵ |
| H3 | 1.0 × 10⁻⁴ |

### Example 1

On an electrode composed of ITO with 70 nm in film thickness formed on a glass substrate, a 10-nm film of the compound E42 was formed with a vacuum degree of 4.0×10⁻⁵ Pa as an electron blocking layer. Then, 2Ph-BTBT, F6-SubPc-OC6F5, and fullerene (C60) were co-deposited at a deposition rate ratio of 4:4:2 with 200 nm to form a film as a photoelectric conversion layer. Subsequently, 10-nm of dpy-NDI was deposited to form a hole blocking layer. Finally, an aluminum film was formed with 70 nm in thickness as an electrode to produce a photoelectric conversion device. A current in a dark place (dark current) was 5.1×10⁻¹⁰ A/cm² with the electrodes of ITO and aluminum and with applying a voltage of 2.6 V. When a voltage of 2.6 V was applied and the ITO electrode side was irradiated with light with an LED adjusted to be an irradiation light wavelength of 500 nm and 1.6 μW from a height of 10 cm, a current (bright current) was 3.7×10⁻⁷ A/cm². A contrast ratio was 7.3×10² with applying a voltage of 2.6 V. Table 3 shows the results.

### Examples 2 to 6

Photoelectric conversion devices were produced in the same manner as in Example 1 except that compounds shown in Table 3 were used as the electron blocking layer.

### Comparative Examples 1 to 3

Photoelectric conversion devices were produced in the same manner as in Example 1 except that compounds shown in Table 3 were used as the electron blocking layer.

Table 3 shows the results of Examples 1 to 6 and Comparative Examples 1 to 3.

The compounds used in Examples and Comparative Examples are shown below.

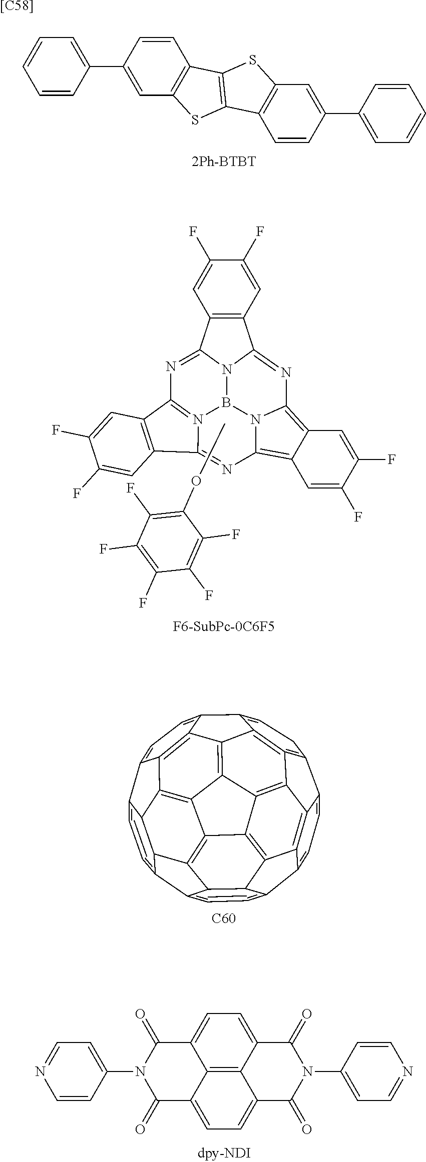

TABLE 3

| | | | Current value | |
|---|---|---|---|---|
| | | Current value | during light | |
| | | in dark place | irradiation | Contrast |
| | Compound | [A/cm²] | [A/cm²] | ratio |
| Example 1 | E42 | 5.1 × 10⁻¹⁰ | 3.7 × 10⁻⁷ | 7.3 × 10² |
| Example 2 | A04 | 4.8 × 10⁻¹⁰ | 3.2 × 10⁻⁷ | 6.7 × 10² |
| Example 3 | C13 | 3.3 × 10⁻¹⁰ | 3.0 × 10⁻⁷ | 9.1 × 10² |
| Example 4 | E45 | 3.2 × 10⁻¹⁰ | 3.5 × 10⁻⁷ | 1.1 × 10³ |
| Example 5 | E126 | 2.9 × 10⁻¹⁰ | 3.6 × 10⁻⁷ | 1.2 × 10³ |
| Example 6 | F125 | 4.6 × 10⁻¹⁰ | 3.8 × 10⁻⁷ | 8.3 × 10² |
| Comparative | H1 | 7.5 × 10⁻¹⁰ | 2.5 × 10⁻⁷ | 3.3 × 10² |
| Example 1 | | | | |
| Comparative | H2 | 6.3 × 10⁻¹⁰ | 3.0 × 10⁻⁷ | 4.8 × 10² |
| Example 2 | | | | |
| Comparative | H3 | 5.5 × 10⁻¹⁰ | 3.2 × 10⁻⁷ | 5.8 × 10² |
| Example 3 | | | | |

### REFERENCE SIGNS LIST

1 Electrode
2 Hole blocking layer
3 Photoelectric conversion layer
4 Electron blocking layer
5 Electrode
6 Substrate

What is claimed is:

1. A material for a photoelectric conversion device, the material comprising a compound represented by the following general formula (1): 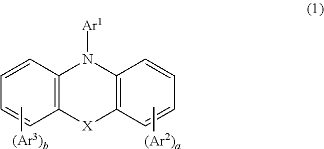
wherein Ar¹ to Ar³ each independently represent deuterium, a cyano group, a halogen, a nitro group, an alkyl group having 1 to 20 carbon atoms, an aralkyl group having 7 to 38 carbon atoms, an alkenyl group having 2 to 20 carbon atoms, an alkynyl group having 2 to 20 carbon atoms, an acyl group having 2 to 20 carbon atoms, an acyloxy group having 2 to 20 carbon atoms, an alkoxy group having 1 to 20 carbon atoms, an alkoxycarbonyl group having 2 to 20 carbon atoms, an alkoxycarbonyloxy group having 2 to 20 carbon atoms, an alkylsulfonyl group having 1 to 20 carbon atoms, a dialkylamino group having 2 to 20 carbon atoms, a substituted or unsubstituted alkylarylamino group having 7 to 28 carbon atoms, a substituted or unsubstituted diarylamino group having 12 to 36 carbon atoms, a substituted or unsubstituted arylheteroarylamino group having 12 to 36 carbon atoms, a substituted or unsubstituted diheteroarylamino group having 12 to 36 carbon atoms, a substituted or unsubstituted aromatic hydrocarbon group having 6 to 18 carbon atoms, a substituted or unsubstituted aromatic heterocyclic group having 3 to 18 carbon atoms, or a substituted or unsubstituted linked aromatic group in which 2 to 10 aromatic rings of the aromatic hydrocarbon group or the aromatic heterocyclic group are linked, at least one of Ar¹ to Ar³ is represented by any of the following general formulae (2) to (8); when Ar¹ to Ar³ represent a group having a hydrogen atom, the hydrogen atom is optionally replaced with deuterium or a halogen,
X represents C(Ar⁴Ar⁵), NAr⁶, O, or S,
“a” and “b” represent the number of substitutions, and each independently represent an integer of 0 to 4, 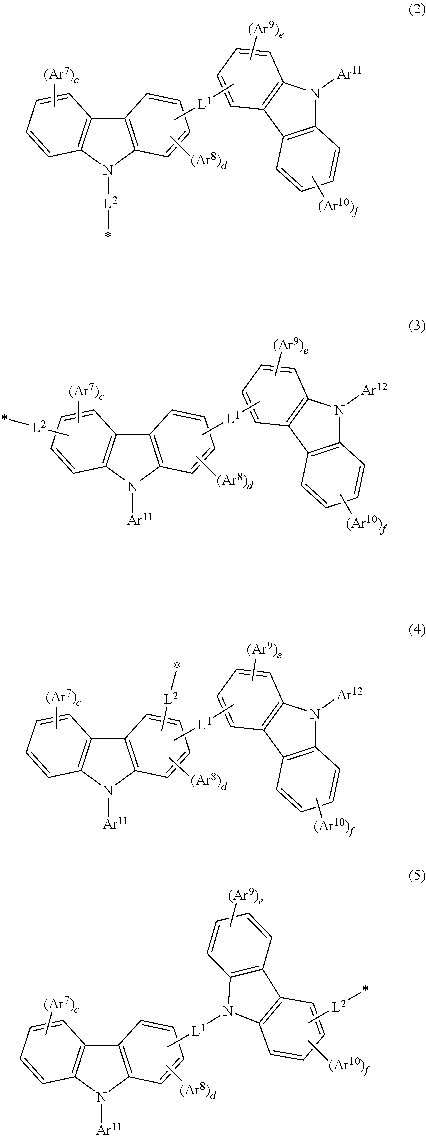 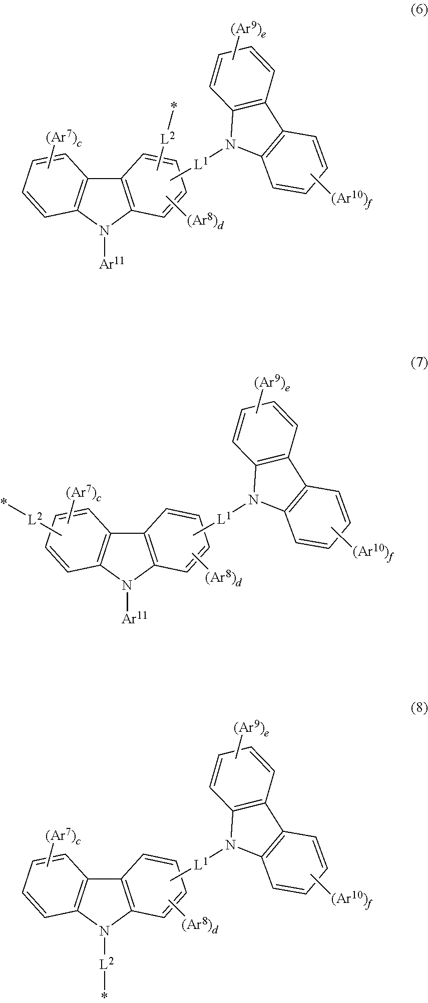
wherein “*” represents a bonding point to the general formula (1),
L¹ and L² each independently represent a direct bond, a substituted or unsubstituted aromatic hydrocarbon group having 6 to 18 carbon atoms, a substituted or unsubstituted aromatic heterocyclic group having 3 to 18 carbon atoms, or a substituted or unsubstituted linked aromatic group in which two to three aromatic rings of these groups are linked,
Ar⁴ to Ar¹² are each independently the same as described for Ar¹ to Ar³, provided that Ar⁴ to Ar¹², when representing the linked aromatic group, represent a substituted or unsubstituted linked aromatic group in which two to three aromatic rings are linked; Ar⁴ and Ar⁵ are optionally bonded to each other to form a cyclic structure,
“c” to “f” represent the number of substitutions, “c”, “e”, and “f” each independently represent an integer of 0 to 3, and “d” represents an integer of 0 to 2.

2. The material for a photoelectric conversion device according to claim 1, wherein the general formula (1) is represented by any of the following general formulae (9) to (12): 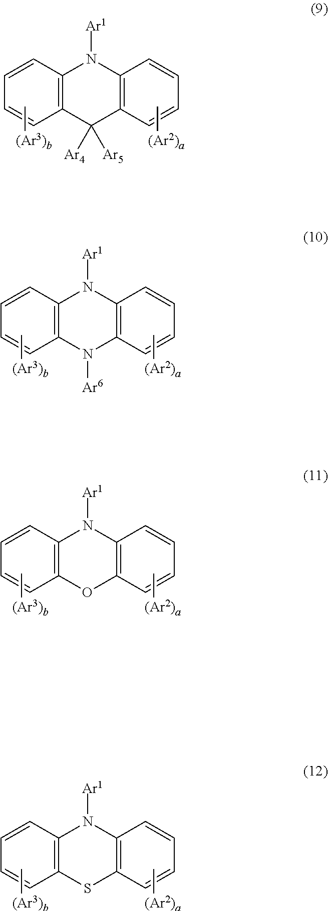
wherein Ar¹ to Ar⁶, “a”, and “b” are the same as described for the general formula (1).

3. The material for a photoelectric conversion device according to claim 2, wherein the general formula (1) is represented by any of the general formulae (11) and (12).

4. The material for a photoelectric conversion device according to claim 1, wherein at least one of Ar¹ to Ar³ in the general formula (1) is represented by any of the general formulae (2), (3), (5), (7), and (8).

5. The material for a photoelectric conversion device according to claim 1, wherein Ar¹ in the general formula (1) is represented by any of the general formulae (2), (3), (5), (7), and (8).

6. The material for a photoelectric conversion device according to claim 1, wherein L¹ represents a single bond.

7. The material for a photoelectric conversion device according to claim 1, wherein an energy level of highest occupied molecular orbital (HOMO) obtained by structural optimization calculation with a density functional calculation B3LYP/6-31G(d) is −4.0 eV or lower.

8. The material for a photoelectric conversion device according to claim 1, wherein an energy level of lowest unoccupied molecular orbital (LUMO) obtained by a structural optimization calculation with a density functional calculation B3LYP/6-31G(d) is −2.5 eV or higher.

9. The material for a photoelectric conversion device according to claim 1, wherein the material has a hole mobility of 1×10⁻⁶ cm²/Vs or more.

10. The material for a photoelectric conversion device according to claim 1, wherein the material is amorphous.

11. The material for a photoelectric conversion device according to claim 1, wherein the material is used as a hole transport material of a photoelectric conversion device for imaging.

12. A photoelectric conversion device for imaging, comprising a photoelectric conversion layer and an electron blocking layer between two electrodes, wherein at least one layer of the photoelectric conversion layer and the electron blocking layer contains the material for a photoelectric conversion device according to claim 1.

13. The photoelectric conversion device according to claim 12, wherein the electron blocking layer contains the material for a photoelectric conversion device according to claim 1.

14. The photoelectric conversion device according to claim 12, wherein the photoelectric conversion layer contains an electron transport material.

15. The photoelectric conversion device according to claim 12, wherein the electron blocking layer contains the material for a photoelectric conversion device according to claim 1, and the photoelectric conversion layer contains a fullerene derivative.
